(19) United States
(12) Patent Application Publication
(10) Pub. No.: US 2026/0308604 A1
(43) Pub. Date: Oct. 8, 2026

(54) BACTERIAL TYPE IV SECRETION SYSTEMS, METHODS OF USE FOR IN VIVO DNA DELIVERY, AND METHOD OF ENGINEERING

(71) Applicant: University of Kentucky Research Foundation, Lexington, KY (US)

(72) Inventors: Carrie L. Shaffer, Lexington, KY (US); Prashant P. Damke, Lexington, KY (US)

(21) Appl. No.: 19/140,685

(22) PCT Filed: Dec. 20, 2023
(86) PCT No.: PCT/US2023/085188

§ 371 Date: Jun. 18, 2025

Related U.S. Application Data
(60) Provisional application No. 63/433,982, filed on Dec. 20, 2022.

(51) Int. Cl. C12N 15/74; A61K 39/00; A61K 39/02; A61K 39/39; C07K 14/205; C12N 1/20; C12N 1/36; C12N 9/22; C12N 15/113; C12R 1/01

(52) U.S. Cl. CPC C12N 15/74; A61K 39/02; A61K 39/39; C07K 14/205; C12N 1/20; C12N 1/36; C12N 9/222; C12N 15/113; A61K 2039/53; A61K 2039/55561; A61K 2039/6006; C12N 2310/20; C12R 2001/01

(57) ABSTRACT

A system for delivering a substrate to a target host cell includes a synthetic Helicobacter pyloricag type IV secretion system (cag T4SS) synthesized within an avirulent bacterial cell and one or more bioactive molecules of interest. The described system includes one or more artificial Helicobacter pyloricag T4SS operons, in embodiments selected from the group consisting of: CagA, CagC, CadD, CagE, CagF, CagG, CagH, CagI, CagM, CagN, CagO, CagP, CagQ, CagS, CagT, CagU, CagV, CagW, CagX, CagY, CagZ, Cag1, Cag2, Cag3, Cag4, Cag5, Cag-alpha. The one or more artificial T4SS operons, when introduced into the avirulent bacterial cell, result in a self-assembling, bi-membrane spanning secretion channel complex adapted to translocate the bioactive molecule to the target host cell. The system is adapted to translocate the substrate to a target host cell, which substrate may be a polynucleotide or protein of interest, DNA, RNA, DNA-RNA hybrids, DNA-encoded molecules, and others.

### GOVERNMENT INTEREST

This invention was made with government support under grant number P20 GM130456 awarded by the National Institutes of Health. The government has certain rights in the invention.

### TECHNICAL FIELD

The presently-disclosed subject matter generally relates to polynucleotide delivery devices. In particular, certain embodiments of the presently-disclosed subject matter relate to the Helicobacter pylori cag type IV secretion system (cag T4SS) as a DNA delivery device that is capable of transferring large fragments of DNA from the bacterial cell to target host cells.

### BACKGROUND

Bacteria have the extraordinary ability to build elaborate nanomachines that deliver molecular cargo to target cells. One versatile apparatus present in nearly all groups of bacteria and some archaeal species is the type IV secretion system (T4SS). These systems are generally contact-dependent and exhibit incredible diversity with respect to the translocated substrate (nucleoprotein complexes, effector proteins, toxins) and cargo destination (prokaryotic or eukaryotic cells, the extracellular milieu). T4SS-dependent mechanisms shape the evolution of bacterial genomes, disseminate antibiotic resistance and virulence determinants throughout microbial populations, and play a central role in bacterial survival within distinct niches, including the human host. Currently, the mechanism by which these molecular machines deliver cargo across the bacterial envelope remains poorly understood, and there is a fundamental lack of understanding of how the T4SS apparatus assembles in response to target cell contact.

My research focuses on discovering how these nanomachines dynamically respond to the host cell to initiate the inter-kingdom transfer of microbial cargo. The present disclosure relates to synthetic T4SS machines that can be fully tuned and controlled in response to external cues. A versatile substrate delivery apparatus is provided within an avirulent bacterium. In effect, a complex T4SS from the gastric bacterium H. pylori is engineered into a substrate delivery tool harbored by an avirulent bacterium. This designer system can be engineered and optimized to deliver proteins, DNA, lipids, polysaccharide complexes, or other bioactive substrates to target cells in vivo. Synthetic T4SSs can be leveraged to guide development of rationally-designed molecules that disrupt transfer of harmful microbial effectors to host cells, or prevent bacterial occupation of specific sites.

### SUMMARY

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes a Helicobacter pylori cag type IV secretion system (cag T4SS) for use as a delivery device for a polynucleotide, such as RNA or DNA. In some embodiments, the device can be used to transfer large fragments of DNA from a bacterial cell to target host cells.

The presently-disclosed subject matter includes a device that makes use of a specific bacterial type IV secretion system that is only found in the human gastric bacterium Helicobacter pylori. Accordingly, the device specifically targets mucosal tissue. For example, H. pylori can deliver full-length genes to human gastric epithelial cells as well as human kidney epithelial cells, where these genes are expressed by the target human cell to produce a protein of interest. Accordingly, the presently-disclosed subject matter is relevant to the fields of drug delivery and therapeutic vaccines.

In some embodiments, the presently-disclosed subject matter provides probiotic bacterial strains to express an optimized secretion system, targeting additional primary tumor sites or tumor microenvironments, and developing a cag T4SS to deliver therapeutic proteins produced by a bacterial cell to target human cells.

The presently-disclosed subject matter includes a modified Helicobacter pylori cag type IV secretion system (cag T4SS), and methods of using such modified system as a polynucleotide delivery device. The device is capable of transferring large fragments of DNA from a bacterial cell to target host cells.

As disclosed herein, H. pylori delivers eukaryotic-optimized, full length genes, comprising eukaryotic gene enhancer elements, promoters, the gene of interest, and polyA tail signals, to human gastric epithelial cells as well as human kidney epithelial cells. These genes are then expressed by the target human cell to produce a protein of interest. Thus, this system can be used to deliver, for example, DNA that encodes vaccine antigens of interest (such as the COVID-19 spike protein) directly to the gastric mucosa. This system can also be used to transfer DNA encoding Cas9 and specific guide RNAs to gastric tumors that would result in the genetic editing or removal of specific human proteins (such as PD-L1) in primary tumors and the tumor microenvironment to enable effective chemotherapy treatment.

The cag T4SS can be engineered as disclosed herein to be pharmacologically controlled (to either turn ON or turn OFF DNA delivery), and H. pylori can be eradicated from the stomach with existing antibiotic therapy or a genetically engineered “kill switch” when treatment/vaccination is no longer indicated.

The cag T4SS can be engineered as disclosed herein to be expressed and function in probiotic bacterial species for delivery to additional intestinal mucosa sites for vaccine delivery and/or in vivo gene editing in target adult tissues as a component of new cancer eradication strategies.

As will be appreciated by one of ordinary skill in the art upon study of this document, the presently-disclosed subject matter beneficially provides for (i) a mucosal-targeted vaccine for the delivery of DNA-encoded antigens of interest to the gastrointestinal mucosa, (ii) in vivo gene editing system for the potential treatment of gastric cancer, and (iii) delivery of specific therapeutic proteins produced by bacterial cells directly into target human cells.

The presently-disclosed subject matter beneficially makes use of a specific bacterial type IV secretion system (cag T4SS) that is only found in the human gastric bacterium Helicobacter pylori. This platform specifically targets mucosal tissue/stomach cells. This system can also be used to genetically edit adult stomach cancer/primary gastric tumors to remove cellular proteins (such as programmed death ligand-1 (PD-L1)) that render current therapies ineffective. This system can be expressed by probiotic bacteria or other bacterial species to target different types of cancers or to deliver specific DNA (such as DNA encoding vaccine antigens) to additional human tissues.

Advantages of the presently-disclosed subject matter include, but are not limited to: (i) targeting stomach mucosal tissue as a vaccination route, (ii) use of a specialized bacterial type IV secretion system as an in vivo vaccine delivery device, (iii) in vivo gene editing in adult tissues to remove cellular features in targeted tissues to increase chemotherapy success rates, (iv) pharmacological control to induce or repress activity of the engineered system, (v) ability to eradicate/remove the bacterial delivery system via antibiotic therapy or genetically encoded “kill switches”.

In some embodiments of the presently-disclosed subject matter, the probiotic bacterial strains are modified to express the optimized secretion system, targeting additional primary tumor sites or tumor microenvironments, developing the cag T4SS to deliver therapeutic proteins produced by the bacterial cell to target human cells.

In one aspect of this disclosure, a system for delivering a substrate to a target host cell is described, comprising a synthetic Helicobacter pylori cag type IV secretion system (cag T4SS) synthesized within an avirulent bacterial cell and one or more bioactive molecules of interest. The described system includes one or more artificial Helicobacter pylori cag T4SS operons, in embodiments selected from the group consisting of: CagA, CagC, CadD, CagE, CagF, CagG, CagH, CagI, CagM, CagN, CagO, CagP, CagQ, CagS, CagT, CagU, CagV, CagW, CagX, CagY, CagZ, Cag1, Cag2, Cag3, Cag4, Cag5, Cag-alpha. As will be appreciated, the one or more artificial T4SS operons, when introduced into the avirulent bacterial cell, result in a self-assembling, bi-membrane spanning secretion channel complex adapted to translocate the bioactive molecule to the target host cell.

In embodiments, the bioactive molecule may be a polynucleotide or protein of interest. In embodiments, the polynucleotide or protein of interest is a DNA or an RNA. In particular embodiments, the bioactive molecule is one or more DNA-encoded vaccine antigens. In other embodiments, the bioactive molecule is a DNA-encoded Cas protein and one or more guide RNAs. In still other embodiments, the bioactive molecule may be a DNA-RNA hybrid.

In still other embodiments, the bioactive molecule is one or more bacterial antigens. In particular alternative embodiments, the bioactive molecule may be an immunostimulatory bacterial DNA adjuvant, a modified bacterial peptidoglycan, or a bacterial-derived cyclic signaling molecule.

Disclosed are the components to be used to prepare the system of the invention as well as the compositions themselves. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D. C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1. H. pylori cag T4SS activity stimulates multiple DNA surveillance systems. A. TLR9 activation induced by the indicated H. pylori 26695 isogenic mutant strain. Data are expressed as the normalized fold change over mock infected cells. B. TLR9 activation requires a functional cag T4SS. C. cGAS-STING signaling stimulated by the indicated strain. D. Induction of double-stranded DNA breaks in the host genome does not significantly contribute to H. pylori-induced cGAS-STING signaling. Graph depicts IFN-β reporter activity induced in cGAS-STING reporter cells by the indicated strain. E. Transwell cGAS-STING activation assays demonstrating the requirement for direct bacteria-host cell contact. F. STING transactivation assays providing evidence of intercellular cGAMP transfer. Schematic depicts the reporter cell line experimental strategy. G. IFN-β transcriptional reporter assays demonstrating the requirement of the cGAS DNA-binding domain (cGAS_{ΔDBD}) and cGAS catalytic activity (cGAS_{ΔNTase}) for H. pylori-induced cGAS-STING signaling. H. cGAS-STING signaling induced by the indicated H. pylori strain stratified by disease state (gastritis, duodenal ulcer, and gastric adenocarcinoma). In A-G, significance was determined by one-way ANOVA with Dunnett's post-hoc correction for multiple comparisons to experimental controls. In all panels, ****, p<0.0001.

FIG. 2. H. pylori DNA is delivered to the gastric epithelial cell cytoplasm in a cag T4SS-dependent manner. A. Representative PCR amplifications demonstrating the presence of chromosomal H. pylori (hp1421), nuclear genomic (hNuc (Fernandez-Moreno et al., 2016)), and mitochondrial (coxII (Fernandez-Moreno et al., 2016)) DNA fragments in fractionated cytoplasmic (C) and total (T) co-culture AGS cell extracts. B. qPCR analysis of H. pylori DNA enrichment in cytosolic fractions normalized to levels of cytosolic mitochondrial DNA. Results are representative of at least 4 biological replicate experiments. Significance was determined by unpaired, two-tailed t-test; **, p<0.01. C. Levels of extracellular cGAMP produced by AGS cells in response to H. pylori. D. Confocal microscopy analysis of perinuclear STING localization in H. pylori-challenged primary gastric epithelial cells at 6 h post-infection. Representative image of n=2 biological replicate experiments depicting STING (green), nuclei (blue), and actin (magenta) staining. Scale bar represents 20 μm. E. Quantitation of STING particle size in primary gastric epithelial cells challenged by the indicated H. pylori strain. Data represents the average STING particle size per field of view for mock infected (7 fields of view, n=90 cells); WT infected (11 fields of view, n=82 cells); and cagX infected (9 fields of view, n=59 cells) gastric epithelial cells. F. STING signaling induced by the indicated H. pylori strain or purified H. pylori chromosomal DNA in 293T reporter cells. Data is representative of a minimum of 3 biological replicate experiments. In E-F, significance was determined by one-way ANOVA with Dunnett's post-hoc correction for multiple comparisons to experimental controls; ****, p<0.0001.

FIG. 3. H. pylori regulates nucleic acid reconnaissance pathways via Cag T4SS activity. A. Volcano plot depicting expression of immune-related genes in adult human primary epithelial cells challenged by H. pylori detected with the NanoString human host response panel. Graph represents the fold change and associated p-value of all differentially expressed genes in the panel for WT vs. cagX challenged cells. Dashed lines dashed lines demarcate genes meeting the threshold for significance (p_{adj}<0.01 and p_{adj}<0.05) after correction with the Benjamini-Hochberg procedure for controlling FDR. Selected genes encoding nucleic acid sensing pathways, interferon-responsive elements, and inflammatory cytokines/chemokines are labeled and indicated in pink. B. Heat map of differentially expressed genes depicted in a. Map depicts genes that were increased or decreased by 1.8-fold and an adjusted p-value <0.01. C. Pathway analysis of differentially expressed immune genes. Graph depicts the-log₁₀ p-value for the indicated pathway. D. TLR7 activation levels induced by the indicated strain or pharmacological stimulus. E. Levels of RIG-I signaling stimulated by H. pylori or transfected RNA agonist. In D and E, significance was determined by one-way ANOVA with Dunnett's post-hoc correction for multiple comparisons to experimental controls; ****, p<0.0001. Data is derived from n=1 NanoString analysis with gastric epithelial cell samples derived from n=2 biological replicate experiments (mock infected, n=3, WT infected n=6, and cagX infected n=3 samples analyzed).

FIG. 4. H. pylori effector DNA is packaged into exosomes to enable DNA pattern recognition receptor signaling in bystander cells. A. TLR9 stimulation induced by supernatants obtained from primary gastric epithelial cells challenged by H. pylori at the indicated time point post-infection. Graph depicts levels of TLR9 activation achieved by supernatants collected in a minimum of four biological replicate experiments. B. Linear regression analysis revealing no correlation between levels of TLR9 activation induced by gastric epithelial cell supernatants and the corresponding level of supernatant total cell-free DNA. C. Levels of TLR9 activation achieved by gastric cell supernatants obtained at 6 h post-infection and processed by the indicated conditions. D. Induction of TLR9 stimulation by gastric cell supernatant extracellular vesicles in gastric epithelial cell supernatants challenged by H. pylori in the presence or absence of GW 4869 (10 μM). Significance was determined by unpaired, two-tailed t-test; ****, p<0.0001. E. qPCR analysis of H. pylori DNA enrichment in purified exosomes derived from primary gastric epithelial co-culture supernatants 6 h post-bacterial challenge by the indicated strain. Graph depicts the fold enrichment of H. pylori DNA (hp1421) over levels of mitochondrial DNA (coxII) in exosomes purified from supernatants collected from five biological replicate experiments. Significance was determined by unpaired, two-tailed t-test; **, p<0.01. F. Schematic representing a proposed model of translocated H. pylori DNA packaging and subsequent release of extracellular vesicles by primary gastric cells. In A and C, significance was determined by one-way ANOVA with Dunnett's post-hoc correction for multiple comparisons to experimental controls; ****, p<0.0001, ***, p<0.001, and **, p<0.01.

FIG. 5. Chromosomally-derived DNA is translocated into target cells via cag T4SS mechanisms. A. Merged tracks of mapped H. pylori DNA reads obtained from infected 293T cGAS cells. Graph depicts sequencing read depth versus nucleotide position in the H. pylori 26695 genome. Schematic illustrates the experimental workflow for cGAS ‘ChIP-seq’ studies. B. DNA reads co-purified with cGAS were normalized to reads obtained from mock infected cells, and peak calling was used to identify regions of bacterial DNA that were enriched with cGAS immunopurification. Dots depict individual peaks and the corresponding peak center on the H. pylori 26695 chromosome obtained from WT (purple dots) and cagX (maroon dots) challenged co-cultures. C. Schematic of eukaryotic-optimized nanoluciferase expression constructs inserted into the hp0073 locus. Nanoluciferase constructs were inserted frameshifted in the opposite orientation of the native operon transcription. D, E. Nanoluciferase bioluminescence produced by AGS (D) and 293T (E) cells challenged by the indicated strain at 24 h post-infection. Data represent a minimum of four biological replicate experiments. Significance was determined by unpaired, two-tailed t-test; **, p<0.01. (F) Low magnification live cell phase contrast (right) and fluorescence microscopy (left) analysis of AGS cells challenged by WT H. pylori harboring LifeAct-mNeonGreen expression constructs at 18 h post-infection. Scale bar represents 100 μm. (G) Representative high magnification images of AGS cells challenged by WT H. pylori harboring LifeAct-mNeonGreen (left) or LifeAct-mScarlet (right). H. pylori (Hp) is stained by anti-H. pylori antibodies. Images in F and G are representative of n=2 biological replicate experiments. H-K. Flow cytometry analysis of AGS (H and I) or 293T cells (J and K) challenged by WT[m Scarlet] or cagX[mScarlet] at 18 h post-infection. Green boxes indicate gating of mScarlet positive cells. Data is representative of n=2 biological replicate experiments.

FIG. 6. DNA translocation is mechanistically coupled to chromosomal replication and replichore decatenation. A. TLR9 activation induced by WT H. pylori in the presence of the indicated ciprofloxacin minimum inhibitory concentration (1×MIC=0.125 μg/ml). Data are expressed as a percent of TLR9 stimulation achieved by WT in mock treated wells. B. Levels of TLR9 activation induced by the indicated isogenic mutant. Data are expressed as a percent of TLR9 stimulation achieved by the parental WT strain. C. IL-8 secreted by AGS cells challenged by the indicated H. pylori strain at 4.5 h post-infection. Data are expressed as a percent of IL-8 levels stimulated by the corresponding WT strain. D. cGAS-9 STING signaling induced by the indicted strain. Graph depicts the results of n=3 biological replicate experiments. E. Transfer DNA immunopurification assays demonstrating the presence of H. pylori chromosomal DNA fragments within the cag T4SS apparatus. Graph depicts the amplification efficiency of a 795 bp fragment in transfer DNA assay preparations purified from the indicated strain. Amplification efficiency of the immunopurification (IP) samples are expressed as the percent of the input DNA from at least four biological replicate experiments. Inset depicts representative PCR amplifications obtained from input and IP samples prepared from the indicated strain. F. Proposed model of DNA cargo segregation for cag T4SS-dependent delivery to host cells. We hypothesize that FtsK-XerH complexes mediate the rare excision of DNA arising from overlapping rounds of chromosomal replication for subsequent coupling to the cag T4SS apparatus for delivery to host cells. In A-E, significance was determined by one-way ANOVA with Dunnett's post-hoc correction for multiple comparisons to experimental controls; ****, p<0.0001 in all panels.

FIG. 7. H. pylori cag T4SS activity stimulates multiple DNA surveillance systems. A. TLR9 activation induced by the indicated H. pylori 26695 isogenic mutant strain. Data are expressed as the normalized fold change over mock infected cells. B. TLR9 activation requires a functional cag T4SS. C. cGAS-STING signaling stimulated by the indicated strain. D. Induction of double-stranded DNA breaks in the host genome does not significantly contribute to H. pylori-induced cGAS-STING signaling. Graph depicts IFN-β reporter activity induced in cGAS-STING reporter cells by the indicated strain. E. Transwell cGAS-STING activation assays demonstrating the requirement for direct bacteria-host cell contact. F. STING transactivation assays providing evidence of intercellular cGAMP transfer. Schematic depicts the reporter cell line experimental strategy. G. IFN-β transcriptional reporter assays demonstrating the requirement of the cGAS DNA-binding domain (cGAS_{ΔDBD}) and cGAS catalytic activity (cGAS_{ΔNTase}) for H. pylori-induced cGAS-STING signaling. H. cGAS-STING signaling induced by the indicated H. pylori strain stratified by disease state (gastritis, duodenal ulcer, and gastric adenocarcinoma). In A-G, significance was determined by one-way ANOVA with Dunnett's post-hoc correction for multiple comparisons to experimental controls. In all panels, ****, p<0.0001.

FIG. 8. H. pylori DNA is delivered to the gastric epithelial cell cytoplasm in a cag T4SS-dependent manner. A. Representative PCR amplifications demonstrating the presence of chromosomal H. pylori (hp1421), nuclear genomic (hNuc (Fernandez-Moreno et al., 2016)), and mitochondrial (coxII (Fernandez-Moreno et al., 2016)) DNA fragments in fractionated cytoplasmic (C) and total (T) co-culture AGS cell extracts. B. qPCR analysis of H. pylori DNA enrichment in cytosolic fractions normalized to levels of cytosolic mitochondrial DNA. Results are representative of at least 4 biological replicate experiments. Significance was determined by unpaired, two-tailed t-test; **, p<0.01. C. Levels of extracellular cGAMP produced by AGS cells in response to H. pylori. D. Confocal microscopy analysis of perinuclear STING localization in H. pylori-challenged primary gastric epithelial cells at 6 h post-infection. Representative image of n=2 biological replicate experiments depicting STING (green), nuclei (blue), and actin (magenta) staining. Scale bar represents 20 μm. E. Quantitation of STING particle size in primary gastric epithelial cells challenged by the indicated H. pylori strain. Data represents the average STING particle size per field of view for mock infected (7 fields of view, n=90 cells); WT infected (11 fields of view, n=82 cells); and cagX infected (9 fields of view, n=59 cells) gastric epithelial cells. F. STING signaling induced by the indicated H. pylori strain or purified H. pylori chromosomal DNA in 293T reporter cells. Data is representative of a minimum of 3 biological replicate experiments. In E-F, 1209 significance was determined by one-way ANOVA with Dunnett's post-hoc correction for multiple comparisons to experimental controls; ****, p<0.0001.

FIG. 9. H. pylori regulates nucleic acid reconnaissance pathways via cag T4SS activity. A. Volcano plot depicting expression of immune-related genes in adult human primary epithelial cells challenged by H. pylori detected with the NanoString human host response panel. Graph represents the fold change and associated p-value of all differentially expressed genes in the panel for WT vs. cagX challenged cells. Dashed lines demarcate genes meeting the threshold for significance (padj<0.01 and padj<0.05) after correction with the Benjamini-Hochberg procedure for controlling FDR. Selected genes encoding nucleic acid sensing pathways, interferon-responsive elements, and inflammatory cytokines/chemokines are labeled and indicated in pink. B. Heat map of differentially expressed genes depicted in a. Map depicts genes that were increased or decreased by 1.8-fold and an adjusted p-value <0.01. C. Pathway analysis of differentially expressed immune genes. Graph depicts the-log10 p-value for the indicated pathway. D. TLR7 activation levels induced by the indicated strain or pharmacological stimulus. E. Levels of RIG-I signaling stimulated by H. pylori or transfected RNA agonist. In D and E, significance was determined by one-way ANOVA with Dunnett's post-hoc correction for multiple comparisons to experimental controls; ****, p<0.0001. Data is derived from n=1 NanoString analysis with gastric epithelial cell samples derived from n=2 biological replicate experiments (mock infected, n=3, WT infected n=6, and cagX infected n=3 replicate samples analyzed).

FIG. 10. H. pylori effector DNA is packaged into exosomes to enable pattern recognition receptor signaling in bystander cells. A. TLR9 stimulation induced by supernatants obtained from primary gastric epithelial cells challenged by H. pylori at the indicated time point post-infection. Graph depicts levels of TLR9 activation achieved by supernatants collected in a minimum of four biological replicate experiments. B. Linear regression analysis revealing no correlation between levels of TLR9 activation induced by gastric epithelial cell supernatants and the corresponding level of supernatant total cell-free DNA. C. Levels of TLR9 activation achieved by gastric cell supernatants obtained at 6 h post-infection and processed by the indicated conditions. D. TLR9 stimulation by gastric cell supernatant extracellular vesicles in gastric epithelial cell supernatants challenged by H. pylori in the presence or absence of GW4869 (10 μM). Significance was determined by unpaired, two-tailed t-test; ****, p<0.0001. E. qPCR analysis of H. pylori DNA enrichment in purified exosomes derived from primary gastric epithelial co-culture supernatants 6 h post-bacterial challenge by the indicated strain. Graph depicts the fold enrichment of H. pylori DNA (hp1421) over levels of mitochondrial DNA (coxII) in exosomes purified from supernatants collected from five biological replicate experiments. Significance was determined by unpaired, two-tailed t-test; **, p<0.01. F. Schematic representing a proposed model of translocated H. pylori DNA packaging and subsequent release of extracellular vesicles by primary gastric cells. In A and C, significance was determined by one-way ANOVA with Dunnett's post-hoc correction for multiple comparisons to experimental controls; ****, p<0.0001, ***, p<0.001, and **, p<0.01.

FIG. 11. Chromosomal DNA is translocated into target cells via cag T4SS mechanisms. A. Merged tracks of mapped H. pylori DNA reads obtained from infected 293T cGAS cells. Graph depicts sequencing read depth versus nucleotide position in the H. pylori 26695 genome. Schematic illustrates the experimental workflow for cGAS ‘ChIP-seq’ studies. B. DNA reads co-purified with cGAS were normalized to reads obtained from mock infected cells, and peak calling was used to identify regions of bacterial DNA that were enriched with cGAS immunopurification. Dots depict individual peaks and the corresponding peak center on the H. pylori 26695 chromosome obtained from WT (purple dots) and cagX (maroon dots) challenged co-cultures. C. Schematic of eukaryotic-optimized nanoluciferase expression constructs inserted into the hp0073 locus. Nanoluciferase constructs were inserted frameshifted in the opposite orientation of the native operon transcription. D, E. Nanoluciferase bioluminescence produced by AGS (D) and 293T (E) cells challenged by the indicated strain at 24 h post-infection. Data represent a minimum of four biological replicate experiments. Significance was determined by unpaired, two-tailed t-test; **, p<0.01. (F) Low magnification live cell phase contrast (right) and fluorescence microscopy (left) analysis of AGS cells challenged by WT H. pylori harboring LifeAct-mNeonGreen expression constructs at 18 h post-infection. Scale bar represents 100 μm. (G) Representative high magnification images of AGS cells challenged by WT H. pylori harboring LifeAct-mNeonGreen (left) or LifeAct-mScarlet (right). H. pylori (Hp) is stained by anti-H. pylori antibodies. Images in F and G are representative of n=2 biological replicate experiments. H-K. Flow cytometry analysis of AGS (H and I) or 293T cells (J and K) challenged by WT[mScarlet] or cagX[mScarlet] at 18 h post-infection. Green boxes indicate gating of mScarlet positive cells. Data is representative of n=2 biological replicate experiments.

FIG. 12. DNA translocation is mechanistically coupled to chromosomal replication and replichore decatenation. A. TLR9 activation induced by WT H. pylori in the presence of the indicated ciprofloxacin minimum inhibitory concentration (1×MIC=0.125 μg/ml). Data are expressed as a percent of TLR9 stimulation achieved by WT in mock treated wells. B. Levels of TLR9 activation induced by the indicated isogenic mutant. Data are expressed as a percent of TLR9 stimulation achieved by the parental WT strain. C. IL-8 secreted by AGS cells challenged by the indicated H. pylori strain at 4.5 h post-infection. Data are expressed as a percent of IL-8 levels stimulated by the corresponding WT strain. D. TLR9 stimulation induced by H. pylori at various growth phases as indicated by culture OD600. Graph depicts the results of n=3 biological replicate experiments. Levels of TLR9 activation inversely correlate to bacterial growth phase (non-linear regression, solid line; 95% confidence intervals, dashed lines). E. Transfer DNA immunopurification assays demonstrating the presence of H. pylori chromosomal DNA fragments within the cag T4SS apparatus. Graph depicts the amplification efficiency of a 795 bp fragment in transfer DNA assay preparations purified from the indicated strain. Amplification efficiency of the immunopurification (IP) samples are expressed as the percent of the input DNA from at least four biological replicate experiments. Inset depicts representative PCR amplifications obtained from input and IP samples prepared from the indicated strain. F. Proposed model of DNA cargo segregation for cag T4SS-dependent delivery to host cells. We hypothesize that FtsK-XerH complexes mediate the rare excision of DNA arising from overlapping rounds of chromosomal replication for subsequent coupling to the cag T4SS apparatus for delivery to host cells. In A-E, significance was determined by one-way ANOVA with Dunnett's post-hoc correction for multiple comparisons to experimental controls; **** *, p<0.0001 in all panels.

FIG. 13. VirD2 ortholog co-option by the cag T4SS facilitates trans-kingdom DNA delivery. A. TLR9 activation is partially susceptible to exogenous nucleases. B. cag T4SS-dependent TLR9 activation is impaired in the presence of monoclonal antibodies targeting dsDNA and ssDNA, but not DNA: RNA hybrids. C. Anti-BrdU monoclonal antibodies inhibit TLR9 activation by BrdU-labeled H. pylori. D. Domain architectures of A. tumefaciens VirD2 and H. pylori Hp0996. AlphaFold2 predicted structure of Hp0996 endonuclease harboring a putative N-terminal relaxase domain (pink) and C-terminal domain of unknown function (DUF, blue). Hp0996 lacks an identifiable nuclear localization signal (NLS). E. TLR9 stimulation induced by the indicated H. pylori strain. Data are expressed as the normalized fold change over mock infected cells. F. EMSA analysis of Hp0996 biding to dsDNA harboring the tfs3 oriT sequence (schematic). Hp0996 and the Hp0996 C-terminal domain bind dsDNA. G. DNA cleavage assays demonstrating Hp0996 relaxase domain nuclease activity. Cleavage assays were performed on dsDNA containing the tfs3 oriT sequence. H. The Hp0996 relaxase domain nicks and linearizes plasmid DNA at the predicted tfs3 oriT sequence. I. Bacterial two-hybrid analysis of Hp0996 protein-protein interactions. Representative images of reporter strains grown on IPTG- and X-Gal-supplemented LB plates are depicted. J. Bacterial two-hybrid reporter strain growth in M63-maltose demonstrating positive or negative interactions among the indicated protein-protein interaction pairs. K. Transfer DNA immunopurification assays demonstrate that Hp0996 and inner membrane ATPases are required for DNA fragment loading into the cag T4SS apparatus. Graph depicts the amplification efficiency of transfer DNA assay preparations purified from the indicated strain. Amplification efficiency of the immunopurification (IP) samples are expressed as the percent of the input DNA from at least four biological replicate experiments. Inset depicts representative PCR amplifications obtained from input and IP samples prepared from the indicated strain. In A-C, E, and J-K, significance was determined by one-way ANOVA with Dunnett's post-hoc correction for multiple comparisons to experimental controls; ****, p<0.0001 in all panels.

FIG. 14. CagC gates the cag T4SS translocation channel. (a) Levels of cag T4SS-dependent TLR9 activation induced by the indicated H. pylori 26695 isogenic mutant strain. Data are expressed as the normalized fold change over mock infected cells as a percent of TLR9 stimulation achieved by WT. (b) Non-denaturing colony blots probing surface-exposed dsDNA, CagA, and H. pylori (Hp) antigens. (c) TLR9 stimulation and (d) IL-8 secretion requires cagC. (e) Levels of viable H. pylori immunocaptured by anti-dsDNA monoclonal antibodies. Data are expressed as the normalized fold change in recovered colony forming units over levels obtained for WT. (f) Erythromycin susceptibility of the indicted strain. Data depict the zone of inhibition for the indicated strain. (g) Transfer DNA immunopurification assays demonstrating the presence of H. pylori chromosomal DNA fragments within the cag T4SS apparatus. Graph depicts the amplification efficiency of a 795 bp fragment in transfer DNA assay preparations purified from the indicated strain. Amplification efficiency of immunopurified samples is expressed as a percent of the input DNA amplification from at least four biological replicate experiments. In a and c-g, significance was determined by one-way ANOVA with Dunnett's post-hoc correction for multiple comparisons to experimental controls; *, p<0.05, **, p<0.01, ****, p<0.0001.

FIG. 15. CagC is a VirB2-like T4SS pilin ortholog. (a) Superimposed model of CagC, VirB2 (T-pilus, PDB: 8CUE), and TraA (F-pilus, PDB: 5LEG). Structures of mature CagC were predicted using AlphaFold2, and the highest-ranked structure was superimposed with canonical pilin structures using the Matchmaker function in ChimeraX. (b) Colony blots demonstrating dsDNA and CagA cell surface localization in H. pylori cagC mutants complemented by either traA, virB2, or the CagC^{R76A} variant. (c) Structures of VirB2 (PDB: 8CUE) and CagC (AlphaFold2 modeling) highlighting Arg 91 (VirB2) and Arg 76 (CagC) residues in the pilin luminal loop region (pink coloring). (d) Western blot analysis of H. pylori cagC mutants genetically complemented by CagC^{WT} or the CagC^{R76A} variant. OMP, outer membrane protein. (e) Levels of TLR9 stimulation and (f) IL-8 secretion induced by the indicated strain. (g) Bacterial two-hybrid analysis of CagC protein-protein interactions. Data depict the fold change in the OD₆₀₀ of E. coli BTH101 cultures transformed with the indicated plasmid pairs and propagated in M63-maltose. Positive protein-protein interactions are indicated by growth in M63-maltose and data are expressed as the fold change in growth over BTH101 transformed with empty vectors (EVs). In a, e and f, significance was determined by one-way ANOVA with Dunnett's post-hoc correction for multiple comparisons to experimental controls; ****, p<0.0001.

FIG. 16. Asymmetric units within the cag T4SS PRC bind dsDNA. (a) EMSA analysis of recombinant MBP-CagC binding to 58-mer dsDNA (left) or ssDNA (right). Arrows indicate recombinant CagC-DNA complexes. (b) The position of CagX subunits within the PRC (PDB: 6X6J). (c) Structure of adjacent CagX subunits (CagX_{NT}, residues 41-310) within the assembled PRC. (d) EMSA analysis of recombinant CagX binding to dsDNA (upper panel) and ssDNA (lower panel). MBP-CagX, but not MBP or MBP-CagT, binds target dsDNA and ssDNA. (e) Quantitation of MBP-CagX binding to the indicated DNA substrate. Values indicate the experimentally approximated K_{D} for CagX-DNA binding to 58-mer targets. (f) Competition assays demonstrating preferential CagX binding to dsDNA. Data points represent the % displaced DNA obtained in independent biological replicate experiments. Solid line represents the nonlinear regression estimating the inhibitory constants (Kᵢ) for dsDNA and ssDNA. In e, dissociation constants (K_{D}) were estimated using the specific binding with hill slope nonlinear regression model and in f, inhibitory constants (Kᵢ) were estimated using the one site fit Kᵢ model (heterologous ligand binding affinities using experimentally derived K_{D} in e) in GraphPad Prism 9.5.

FIG. 17. Symmetry mismatch coordinates DNA substrate selection for trans-kingdom conjugation. (a) Electrostatic surface potential of two adjacent CagX residues demonstrating the formation of a basic patch at the intersubunit junction. Regions of acidic and basic potential are shown in red and blue, respectively. (b) Overlapping pi-pi orbital interactions among CagX arginine and tryptophan residues in adjacent subunits. (c) Proposed model of dsDNA binding to CagX subassemblies. (d) Quantitation of CagX variant binding to dsDNA. Experimentally approximated K_{D} for CagX^{WT} (1.7 μM), Cag^{XR36A,R38A} (1.9 μM), CagX^{R278A} (3.7 μM), and CagX^{R36A,R38A,R278A} (4.6 μM) were calculated for 58-mer target binding. (e) Levels of TLR9 activation and (f) IL-8 secretion stimulated by H. pylori harboring the indicated CagX variant. In d, dissociation constants (K_{D}) were estimated using the specific binding with hill slope nonlinear regression model in GraphPad Prism 9.5. In e and f, significance was determined by one-way ANOVA with Dunnett's post-hoc correction for multiple comparisons to experimental controls; ****, p<0.0001.

FIG. 18. Proposed model of DNA substrate selection within the cag T4SS PRC. (a) Symmetry mismatch between CagX subunits in the OMC (upper panel, 14-fold symmetry) and the PRC (lower panel, 17-fold symmetry) single particle cryo-EM reconstruction. (b) Positions of CagY (yellow) and CagX (orange) within the cag T4SS OMC and the PRC. (c) Illustration of one asymmetric CagX connection between the OMC and PRC (dashed circle). Three copies of CagX in the PRC do not show an obvious connection to corresponding CagX subunits in the OMC. (d) Proposed model of dsDNA loading into the cag T4SS core complex via electrostatic interactions and pi-pi stacking within asymmetric PRC units. Illustration depicts the architectural juxtaposition of CagX N-terminal domains (CagX_{NT}), CagX C-terminal domains (CagX_{CT}), CagY N-terminal domains (CagY_{NT}), and CagY C-terminal domains (CagY_{CT}) in the OMC and PRC with dsDNA loading into the secretion channel via sub-complex symmetry mismatch.

FIG. 19. CagC structural modeling. (a) Protein sequence alignment of prototypical T4SS pilins VirB2 (A. tumefaciens vir T4SS), TraA (E. coli F pilus pED208), and CagC. Pilin sequence alignment was generated using MegAlign Pro. (b) Modeling scores determined by AlphaFold2 for the highest-ranked CagC models (pLDDT, predicted local distance difference test, representing local model accuracy). (c) Heatmap representing the inter-chain accuracy of the highest-rank CagC model. Heatmap pixels indicate alignment error (in Angstroms) for a pair of two residues, indicating the positional uncertainty at residue x if the predicted and actual structures are aligned on residue y.

FIG. 20. CagX preferentially binds target dsDNA. (a) Coomassie analysis of recombinant maltose binding protein (MBP) fusions purified from E. coli.(b) 58-mer dsDNA EMSA analysis of Trak derived from F plasmid R1 (structural model depicts Trak from F plasmid pED208, PDB: 7SPB) and (c) VirB9 derived from A. tumefaciens vir T4SS (structural model depicts VirB9 from X. citri vir T4SS, PDB: 6GYB). MBP-CagX, but not MBP-Trak or MBP-VirB9, binds dsDNA. (d, e) Competitive DNA binding assays demonstrating CagX preferential dsDNA binding. Recombinant CagX was pre-loaded with Cy5-labeled dsDNA (d) or ssDNA (e) and the indicated cold competitor was added at increasing concentrations. (f) Quantitation of displaced DNA in Cy5-ssDNA pre-loaded reactions. Data points represent the % displaced DNA obtained in independent biological replicate experiments. Solid line represents the non-linear regression using the one site fit Kᵢ model in GraphPad Prism 9.5 to estimate the indicated inhibitory constants (Kᵢ) for dsDNA and ssDNA.

FIG. 21. Intersubunit pi-stacking is required for CagX dsDNA binding. (a) Coomassie analysis of recombinant maltose binding protein (MBP) CagX variant fusions purified from E. coli.(b) EMSA analyses of recombinant CagX variant binding to dsDNA. (c) Quantitation of MBP-CagX binding to 58-mer DNA substrates. The experimentally approximated K_{D} for CagX-DNA binding was determined for CagX^{WT} (1.7 μM), CagX^{R278A,R295A} (4.5 MM), CagX^{R95A} (6.9 μM), and CagX^{R36A,R38A,R295A} (5.2 μM). (d) TLR9 stimulation and (e) IL-8 secretion induced by H. pylori strains. (f) Levels of CagX produced by H. pylori cagX harboring the indicated CagX variant. In c, dissociation constants (K_{D}) were estimated using the specific binding with hill slope nonlinear regression model in GraphPad Prism 9.5. In d and e, significance was determined by one-way ANOVA with Dunnett's post-hoc correction for multiple comparisons to controls; ****, p<0.0001.

FIG. 22. Hp0996 and FtsK are required for transfer DNA loading into the H. pylori cag T4SS. TrIP assay demonstrating the presence of H. pylori transfer DNA in the cag T4SS apparatus in the absence of host cell contact in the indicated isogenic mutant. DNA loading requires the structural component CagY, Hp0996, FtsK, and the cag T4SS ATPases CagE and Caga, but not the VirD4-like coupling protein Cag5. DNA loading defects can be genetically complemented with epitope-tagged forms of Hp0996 (Hp0996-HA) or FtsK (Ftsk-GSK). HA, human influenza hemagglutinin; GSK, Glycogen Synthase Kinase 3β.

FIG. 23. FtsK directly interacts with the cag T4SS apparatus. Bacterial two-hybrid reporter strain growth in M63-maltose demonstrating positive or negative interactions among the indicated protein-protein interaction pairs. FtsK interacts with Caga and the inner membrane component Cag V, but not the core complex components CagX or CagT, or ATPases CagE and Cag5.

FIG. 24. Engineered plasmids are translocated from H. pylori to target human cells via cag T4SS activity. Nanoluciferase bioluminescence produced by AGS cells challenged by WT H. pylori G27 harboring the indicated pHel plasmid at 24 h post-infection. Data represent a minimum of four biological replicate experiments. Dashed line indicates a positive signal defined as a three-fold increase over mock infected wells. pHel-NL, NanoLuciferase construct; oriT, NanoLuciferase flanked by the tfs3 oriT consensus sequence at the 5′ region; difH-5′, NanoLuciferase flanked by a difH consensus sequence at the 5′ region; difH-3′, NanoLuciferase flanked by a difH consensus sequence at the 3′ region, difH-oriT, NanoLuciferase flanked by tandem oriT-difH consensus sequences at the 5′ region; oriT-NL-difH, NanoLuciferase flanked by an oriT consensus sequence at the 5′ region and a difH consensus sequence at the 3′ region.

### DETAILED DESCRIPTION

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

All publications, patents, and patent applications mentioned in this specification are herein incorporated by reference to the same extent as if each individual publication, patent, or patent application was specifically and individually indicated to be incorporated by reference.

### Helicobacter pylori Provokes STING Immunosurveillance via Trans-Kingdom Conjugation

Recognition of foreign nucleic acids is an evolutionarily conserved mechanism by which the host detects microbial threats. Whereas some intracellular bacterial pathogens trigger DNA surveillance pathways following phagosomal membrane perturbation, mechanisms by which extracellular bacteria activate cytosolic nucleic acid reconnaissance systems remain unresolved. Here, we demonstrate that Helicobacter pylori exploits cag type IV secretion system (cag T4SS) activity to provoke STING signaling in gastric epithelial cells. We provide direct evidence that chromosomal fragments delivered to the host cell cytoplasm via trans-kingdom conjugation bind and activate the key DNA sensor cGMP-AMP synthase. To enable paracrine-like signal amplification, translocated H. pylori DNA is sorted into exosomes that stimulate DNA-sensing pathways in uninfected bystander cells. We show that DNA cargo is loaded into the cag T4SS apparatus in the absence of host cell contact to establish a ‘ready-to-fire’ nanomachine and provide evidence that cag T4SS-dependent DNA translocation is mechanistically coupled to chromosomal replication and replichore decatenation. Collectively, these studies suggest that H. pylori evolved mechanisms to stimulate nucleic acid surveillance pathways that regulate both pro- and anti-inflammatory programs to facilitate chronic persistence in the gastric niche.

Innate recognition of invariant pathogen-associated molecular signatures by cellular pattern recognition receptors (PRRs) is the first line of defense against microbial adversaries. Within the gastrointestinal tract, epithelial cells express proximal endosomal and cytosolic nucleic acid-sensing PRRs that rapidly respond to aberrant microbial DNA and RNA to trigger innate defense mechanisms and coordinate adaptative immunity. Localization of foreign DNA to the host cell cytosol activates multiple nucleic acid reconnaissance systems including the key DNA sensor nucleotidyltransferase cyclic GMP-AMP synthase (cGAS) (Cai et al., 2014; Diner et al., 2013; Sun et al., 2013; Wu et al., 2013). Upon binding DNA originating from either extrinsic or intrinsic sources, cGAS catalyzes the formation of the non-canonical cyclic dinucleotide 2′3′-cGAMP using ATP and GTP as substrates (Diner et al., 2013; Gao et al., 2013a; Gao et al., 2013b; Sun et al., 2013; Zhang et al., 2013). In turn, 2′3′-cGAMP stimulates the endoplasmic reticulum receptor STING (stimulator of interferon genes) to elicit interferon (IFN) signaling and the production of multifarious inflammatory cytokines (Ishii et al., 2006; Ishikawa and Barber, 2008; Ishikawa et al., 2009; Stetson and Medzhitov, 2006; Sun et al., 2009; Zhong et al., 2008). Dysregulation of mucosal STING signaling can disrupt gut homeostasis and generate pro-tumorigenic inflammatory microenvironments (Ke et al., 2022); however, the outcomes of STING-dependent immune surveillance within the context of gastric inflammation and infection-associated carcinogenesis remain unresolved.

Of the known infection-associated cancers, the most significant carcinogenic microbe is the gastric bacterium Helicobacter pylori, which chronically colonizes the stomach of over half of the global population and directly contributes to the development of more than one million new cases of cancer per year (Sung et al., 2021). H. pylori harboring the cancer-associated cag type IV secretion system (cag T4SS) significantly augment disease risk via translocation of pro-inflammatory molecular cargo into gastric epithelial cells. In addition to facilitating the delivery of the bacterial oncoprotein CagA, the cag T4SS translocates a diverse repertoire of immunostimulatory lipid, nucleic acid, and polysaccharide substrates directly into the gastric epithelium (Amieva and Peek, 2016; Cover et al., 2020). Recent work reported that H. pylori cag T4SS activity activates the endosomal DNA-sensing PRR Toll-like Receptor 9 (TLR9), leading to immune suppression conferring tolerance (Varga et al., 2016a; Varga et al., 2016b) as well as inflammation-independent carcinogenic phenotypes (Castano-Rodriguez et al., 2014). In addition to TLR9, multiple cellular nucleic acid sensors, including cGAS (Ablasser et al., 2013; Cai et al., 2014; Diner et al., 2013; Gao et al., 2015; Nandakumar et al., 2019; Storek et al., 2015; Watson et al., 2015; Zhang et al., 2014), RIG-I (Chow et al., 2015; Dixit and Kagan, 2013; Onomoto et al., 2021; Rad et al., 2009), MDA5 (Dixit and Kagan, 2013; Wu et al., 2020), AIM2 (Rathinam et al., 2010), ZBP1/DAI (Kuriakose et al., 2016), IFI-16 (Almine et al., 2017; Unterholzner et al., 2010), and RNA pol III (Ablasser et al., 2009; Chiu et al., 2009) are expressed in the human gastric epithelium and associated mucosal dendritic cells (Rad et al., 2009), raising the hypothesis that H. pylori stimulates additional innate nucleic acid surveillance pathways.

While intracellular bacterial pathogens elicit cGAS-STING signaling following phagosomal membrane destabilization or rupture achieved in a type III, IV, VI, or VII secretion system-dependent manner (Ku et al., 2020; Marinho et al., 2017; Nandakumar et al., 2019; Storek et al., 2015; Watson et al., 2015; Zhang et al., 2014), the mechanisms by which extracellular bacteria stimulate DNA reconnaissance systems remain unclear. STING signaling has been implicated in gastric carcinogenesis and H. pylori has been shown to activate STING in vivo (Song et al., 2017), but whether H. pylori-driven STING activation requires cytosolic nucleic acid immunosurvellience is unknown. Here, we demonstrate that H. pylori-induced STING signaling is a direct consequence of cag T4SS-dependent DNA translocation. We show that H. pylori chromosomal fragments delivered to the gastric epithelium via trans-kingdom conjugation directly bind and activate cGAS to stimulate STING signaling. We discovered that upon translocation into primary gastric epithelial cells, fragmented H. pylori DNA is sorted into exosomes that are released to amplify foreign nucleic acid immune surveillance in uninfected bystander cells. We provide direct evidence that cag T4SS-mediated DNA translocation is mechanistically coupled to chromosomal replication and demonstrate that eukaryotic-optimized constructs greater than 1.5 kb are delivered to the gastric epithelium via cag T4SS mechanisms. Our results highlight how H. pylori exploits the versatile cag T4SS to tip the delicate STING signaling balance towards inflammatory responses that may stimulate carcinogenesis and enable chronic colonization of the gastric niche.

### Methods

Bacterial strains and culture conditions. Helicobacter pylori strain 26695, strain G27, isogenic derivatives, and clinical isolates were propagated on trypticase soy agar plates supplemented with 5% sheep blood (BD) as previously described (Shaffer et al., 2011). Overnight cultures of H. pylori were grown in Brucella broth supplemented with 5% fetal bovine serum (FBS) at 37 °C with 5% CO₂. For cloning purposes, E. coli DH5α (New England Biolabs) were grown on lysogeny broth (LB) agar plates or in LB liquid medium with appropriate antibiotics required for plasmid maintenance.

Human cell culture. The AGS human gastric adenocarcinoma epithelial cell line (ATCC CRL-1739) was cultured in RPMI 1640 medium supplemented with 10% FBS, 2 mM L-glutamine, and 10 mM HEPES in the presence of 5% CO₂ at 37 °C. Primary adult human gastric epithelial cells (Cell Biologics H-6039) were grown in human epithelial cell medium supplemented with ITS, EGF, hydrocortisone, L-glutamine, and 5% FBS (Cell Biologics H6621) in collagen-coated cell culture flasks at 5% CO₂ and 37 °C. HEK293-hTLR9 (Invivogen hkb-htlr9) and the corresponding parental HEK293 null1 (InvivoGen hkb-null1) cell lines were grown in DMEM media supplemented with 10% heat-inactivated FBS and 1× GlutaMAX (Life Technologies) in the presence of 5% CO₂ at 37 °C. Prior to assays, wells of tissue culture plates were coated in collagen following manufacturer's protocol. HEK293-hTLR9 (Invivogen hkb-htlr9), the corresponding parental HEK293 null1 (InvivoGen hkb-null1), HEK293-hTLR7 (Invivogen hkb-htlr7), HEK-Lucia™ hRIG-I (Invivogen hkl-hrigi), and 293T (ATCC CRL-3216) were grown in DMEM supplemented with 10% heat-inactivated FBS and 1× GlutaMAX (Life Technologies) in the presence of 5% CO₂ at 37 °C. For cell treatments prior to bacterial challenge, the following compounds were added at the indicated final concentration one hour prior to bacterial challenge: MitoTempo (Sigma-Aldrich, 100 μM); N-acetylcysteine (Sigma-Aldrich, 1 mM); BAX-inhibiting peptide, negative control (Sigma-Aldrich, 200 μM); BAX-inhibiting peptide V5 (Sigma-Aldrich, 200 μM); or sphingomyelinase inhibitor GW4869 (Sigma-Aldrich, 10 μM).

H. pylori mutagenesis. Isogenic mutant derivatives of H. pylori 26695 and G27 were generated essentially as previously described (Johnson et al., 2014; Shaffer et al., 2011; Varga et al., 2021). Briefly, H. pylori was transformed with a suicide plasmid in which the coding region of the target gene was replaced by either a kanamycin resistance cassette or a chloramphenicol resistance cassette and homologous flanking DNA sequences up- and downstream of the target locus. Colonies resistant to kanamycin (12.5 μg/ml) or chloramphenicol (10 g/ml) were PCR-verified to confirm insertion of the resistance cassette into the appropriate locus. To complement mutants in cis at the ureA locus, plasmids derived from pAD1 (Shaffer et al., 2011) were constructed to express either the native gene or a hemagglutinin (HA) epitope-tagged protein as previously described (Shaffer et al., 2011; Varga et al., 2021). Plasmid sequences were confirmed by sequencing, and constructs were used to transform isogenic mutant strains. Insertion of complementation constructs into the ureA locus was confirmed by PCR amplification and anti-HA immunoblotting, when appropriate.

To construct H. pylori strains harboring NanoLuc luciferase expression constructs, a 1654 bp fragment encompassing the CMV enhancer element, CMV promoter, NanoLuc luciferase gene, and SV40 late poly(A) signal was amplified from pNL1.1.CMV[Nluc/CMV] (19.1 kDa NanoLuc protein, Promega). The PCR product was blunt end ligated to pAD1 digested with EcoRV. Ligation insertion was confirmed by PCR and DNA sequencing. Clones in which the NanoLuc luciferase construct was inserted in the reverse orientation relative to ureA transcription were selected for transformation into WT and cagX H. pylori 26695 and WT G27 strains. Integrations into the H. pylori chromosome confirmed by PCR. The G27 isogenic cagE mutant was generated by replacement of the cagE coding region with a kanamycin resistance cassette as previously described (Shaffer et al., 2011; Varga et al., 2021). H. pylori strains harboring monomeric LifeAct-mScarlet (Bindels et al., 2017) expression constructs were generated using the same mutagenesis strategy, with the exception that a 1872 bp region of pLifeAct_mScarlet-i_N1 (Bindels et al., 2017) (26.4 kDa LifeAct-mScarlet protein, Addgene) encompassing the CMV enhancer element, CMV promoter, LifeAct-mScarlet gene, and SV40 late poly(A) signal was amplified was cloned into pAD1 via blunt end ligation. Clones in which the LifeAct mScarlet construct was inserted in the reverse orientation relative to ureA transcription were selected for transformation into H. pylori 26695 and G27 strains. Integrations into the ureA locus were confirmed by PCR.

Isogenic mutants^{18,38,48,69} were complemented in cis at the ureA locus using plasmids derived from pAD1⁴⁸ that were designed to express either the native gene or a protein variant as previously described^{48,49}. Point mutants were generated using Q5 Site Directed Mutagenesis (New England Biolabs). Plasmid sequences were confirmed by PCR and whole plasmid sequencing, and constructs were used to transform isogenic mutant strains. Colonies resistant to kanamycin (12.5 μg/ml) or chloramphenicol (10 μg/mL) were selected and complementation at the ureA locus was confirmed by PCR and by anti-CagX immunoblotting, when appropriate.

cGAS-STING reporter assays. 293T cells seeded into 24-well plates at a density of approximately 1.5×10⁵ cells per well were transfected using Lipofectamine 2000 (Life Technologies) complexed with a combination of plasmids pUNO1-hSTING-R232 (Invivogen puno1-hStingWT), pUNO1-hcGAS-HA3X (Invivogen pUNO1-HA-hcGAS), cGAS derivatives (pUNO1-hcGAS-AA, Invivogen pUNO1-hcGAS-AA; pcDNA3.1-cGAS_{ΔDBD}, Addgene 102606), pRL-SV40P (Addgene 27163), IFN-Beta-pGL3 (Addgene 102597), or empty vector pcDNA3.1 (Life Technologies V79020) to ensure equivalent DNA concentrations according to the manufacturer's protocol. At 16 h post-transfection, cells were challenged by mid-log phase WT H. pylori, corresponding cag isogenic mutants, or cag T4SS+ clinical isolates at a multiplicity of infection (MOI) of 20 bacterial cells per 293T cell. Alternatively, STING reporter cells were transfected with purified H. pylori genomic DNA (500 ng/well) at 16 h post-initial transfection of STING and IFN-β reporter plasmids. For cGAS-STING time course assays, H. pylori were added to reporter cell monolayers at a MOI of 20, and culture plates were centrifuged at 1,800×g for 1 min to synchronize infections. At the indicated time point, gentamicin was added at a final concentration of 100 μg/ml.

At 24 h post-infection, cell supernatants were removed and adherent cells were lysed in luciferase assay passive lysis buffer (Pierce). Luciferase luminescence generated by 20 μl cell lysate per well in a 96-well plate format was recorded with a microplate reader (BioTek Synergy H1) using the Dual-Luciferase Reporter Assay System (Pierce). Firefly luciferase luminescence (D-Luciferin, IFN-Beta-pGL3) was normalized to Renilla luciferase luminescence (coelenterzine, pRL-SV40P) for each well, and IFN-β transcriptional reporter values were expressed as the normalized fold change over mock-infected wells. A minimum of three biological replicate experiments were performed in quadruplicate for each assay.

To evaluate the requirement of direct bacteria-host cell contact for cGAS-STING signaling, 293T cells were seeded directly into the well of a 24-well plate or into a 0.4 μm pore size transwell insert at approximately 5×10⁴ cells per transwell. Cells were transfected as described for cGAS-STING reporter cell assays. To physically separate bacterial cells from reporter cells, transfected cGAS-STING reporter cells challenged by the addition of WT H. pylori 26695 added to either the apical transwell chamber (293T cells seeded directly into the cell culture well), or the basolateral compartment (293T cells seeded in the transwell apparatus) at a MOI of 20. As a control, cGAS-STING assays in which H. pylori were added directly to reporter cell monolayers were performed in parallel. After 24 h of bacterial infection, monolayers were processed as described for cGAS-STING activation assays, and luminescence values were obtained. IFN-β transcriptional reporter values were expressed as the normalized fold change over mock infected wells.

STING transactivation assays. For STING transactivation assays, 293T cells were transfected with either (i) pUNO1-hcGAS-HA3X, pRL-SV40P, and pcDNA3.1 empty vector, or (ii) pUNO1-hSTING-R232, pRL-SV40P, and IFN-Beta-pGL3 using Lipofectamine 2000 as described for cGAS-STING activation assays. At 12 h post-transfection, cells were lifted using sterile phosphate buffered saline (PBS) and were re-plated in 24-well dishes at a 1:1 ratio at approximately 2×10⁵ cells/well. Cells were allowed to adhere and were subsequently challenged by the indicated H. pylori strain at a MOI of 20. After 24 h of bacterial infection, IFN-β transcriptional reporter activity was assayed as described for cGAS-STING activation experiments. STING transactivation by cGAMP diffusion was expressed as the normalized fold change of IFN-β transcriptional reporter activity over mock infected wells.

TLR and RIG-I activation assays. To assess TLR9 or TLR7 activation, HEK293 stably transfected with hTLR9, hTLR7, or parental null cells (Invivogen) were seeded into 96-well plates at a density of approximately 2×10⁴ cells per well, and were subsequently by WT H. pylori 26695 or its isogenic cag mutant strains at a MOI of 100 in quadruplicate as previously described (Varga et al., 2016b; Varga et al., 2021). Supernatants were collected at 24 h post-infection, and TLR9 and TLR7 activation was determined by measuring secreted embryonic alkaline phosphatase (SEAP) reporter activity in cell culture supernatants by QuantiBlue™ reagent (Invivogen) using a microplate reader (BioTek Synergy HI) to record the absorbance at 650 nm. As a positive control for TLR7 activation, HEK293-hTLR7 reporter cells were stimulated with 5 μg/ml imiquimod (R837), an imidazoquinoline amine analog to guanosine (Invivogen). TLR9 or TLR7 activation was normalized to SEAP levels produced by infected null1 parental cells and is expressed as the fold-change over mock infected controls. For ciprofloxacin inhibition of bacterial DNA replication, ciprofloxacin was added to HEK293-hTLR9 co-cultures at the same time as bacterial inoculation at multiples of the previously reported minimum inhibitory concentration (MIC), with 1× MIC equivalent to 0.125 μg/ml. Inhibition of TLR9 activation is expressed as a percent of the normalized fold change over mock treated, H. pylori-challenged wells.

For RIG-I activation studies, HEK-Lucia™ hRIG-I cells were plated in a 96-well dish at approximately 5×10⁴ cells per well and were subsequently challenged by WT H. pylori 26695 or the indicated isogenic mutant strain at a MOI of 100 in quadruplicate. As a positive control, cells were transfected with 100 ng/ml of the RIG-I agonist 5′ triphosphate hairpin RNA (3p-hpRNA, Invivogen) complexed to Lipofectamine 2000 (Life Technologies) according to the manufacturer's protocol. At 24 h post-challenge, RIG-I stimulation was assessed by analyzing Lucia luciferase reporter gene expression in 20 μl cell culture supernatants using QUANTI-Luc™ (Invivogen). Data are expressed as the normalized fold change over mock infected cells. TLR and RIG-I stimulation experiments were performed a minimum of three times with quadruplicate technical replicates per experimental condition.

CagA translocation assays. AGS or 293T cells were plated in 12-well dishes at a seeding density of 1×10⁵ cells/well and were cultured overnight. Monolayers were challenged by the indicated H. pylori strain at a MOI of 100 for 6 h, as previously described (Shaffer et al., 2011; Varga et al., 2021). Wells were washed in sterile PBS to remove non-adherent bacteria, and cells were lysed in assay buffer (1% NP-40) supplemented with cOmplete™ mini EDTA-free protease inhibitor (Roche) and PhosSTOP phosphatase inhibitor (Roche). The soluble fraction was collected and prepared in 2× SDS buffer for immunoblotting. To assess CagA tyrosine phosphorylation, AGS or 293T samples were separated on 7.5% gels (Bio-Rad) for 60 min at 165V. Proteins were then transferred onto nitrocellulose using the TransBlot Turbo system (Bio-Rad) following manufacturer's recommendations. Membranes were blocked in 3% BSA in Tris-buffered saline containing 0.1% Tween-20 (TBST), followed by incubation with anti-phosphotyrosine monoclonal antibody (α-PY99, Santa Cruz Biotechnology). Total CagA levels were assessed by subsequent incubation with an anti-CagA monoclonal antibody (α-CagA, Santa Cruz Biotechnology). Phosphorylation of CagA or total CagA was visualized using chemiluminescence (Pierce). TEM-CagA translocation was assayed as previously described (Varga et al., 2021).

Bacterial adherence and internalization assays. Adherence and internalization into gastric or kidney epithelial cells were assessed as previously described (Varga et al., 2021). Briefly, WT H. pylori or the cagX isogenic derivative were co-cultured with AGS or 293T cells at a MOI of 100. After a 4 or 6 h infection, respectively, cell culture medium was aspirated, and cell monolayers were gently washed with sterile PBS to remove non-adherent bacteria. To assess intracellular bacteria, RPMI or DMEM supplemented with gentamycin (100 μg/mL) was added to a subset of wells, and the cells were incubated for an additional hour at 37 °C in 5% CO₂. To assess total adherent and intracellular bacteria, fresh RPMI or DMEM was replenished into the remainder of wells following the removal of non-adherent bacteria. After the 1 h incubation, RPMI or DMEM was aspirated and all wells were washed in sterile PBS, lysed by mechanical disruption, and were serially diluted on blood agar plates for colony enumeration. Experiments were performed a minimum of three times with triplicate technical replicates per cell line and culture condition.

Detection of bacterial DNA in AGS cytosolic extracts. Digitonin extracts of AGS gastric epithelial cells were prepared essentially as previously described (West et al., 2015). Briefly, approximately 8.4×10⁶ AGS cells were infected with exponentially growing WT or cagX H. pylori at a MOI of 50. As a control, an equivalent number of AGS cells were mock infected. After 6 h of infection, cells were rinsed with sterile PBS and were treated with 50 units of Turbo DNasel (Life Technologies) in digestion buffer at 37 °C for 15 min. The cells were rinsed twice with PBS, trypsinized, and collected in 2 ml of sterile PBS. Collected cells were separated into aliquots of approximately 400 μl to generate ‘total’ cell extracts, and of approximately 1600 μl to generate ‘cytosolic’ extracts. Aliquots were centrifuged at 980×g for 3 min and cell pellets were washed once with PBS. To generate the ‘total’ cell extract, one pellet for was re-suspended in 100 μl of 50 μM NaOH and incubated for 30 min at 95 °C to solubilize DNA, followed by pH neutralization by the addition of 10 μl of 1 M Tris-HC 1, pH 8. These extracts served as normalization controls for the quantitation of mitochondrial DNA (mtDNA) and bacterial DNA. To generate ‘cytosolic’ extracts, cell pellets were re-suspended in cytosolic extraction buffer (150 mM NaCl, 50 mM Tris pH 8.0, and 20 μg/ml Digitonin [Sigma-Aldrich]), and homogenates were rotated end-over-end for 10 min at room temperature for selective membrane permeabilization. Cytosolic fractions were separated from intact cells and nuclear/bacterial fractions by serial centrifugations at 17,000×g for 3 min. Recovered supernatants were incubated for 10 min at 95 °C to isolate DNA. To assess the presence of nuclear DNA, mtDNA, and H. pylori DNA in cellular fractions, standard PCR and qPCR targeting fragments of the H. pylori chromosome (hp1421, 290 bp), mtDNA (coxII, 444 bp(Fernandez-Moreno et al., 2016)), and nuclear DNA (hNuc, 467 bp (Fernandez-Moreno et al., 2016)) were amplified from ‘total’ and ‘cytosolic’ fractions using Taq polymerase (standard PCR) or Fast SYBR Green chemistry (qPCR) on a Viia7 platform (Thermo). C_{T} values obtained for cytosolic fractions were normalized to corresponding C_{T} values obtained for total cell extracts, and cytosolic enrichment of bacterial DNA was calculated as the normalized ratio of hp1421 C_{T} values to coxII C_{T} values. A minimum of four biological replicate experiments were performed for each experimental condition.

Quantitation of secreted extracellular cGAMP. AGS cells seeded into T25 flasks were grown to approximately 80% confluency were mock infected or were challenged by H. pylori at an MOI of 100 for 6 h. Bacteria were inactivated by the addition of 200 μg/ml gentamicin, and monolayers were incubated overnight. At 24 h post-infection, equivalent volumes of cell culture supernatants were collected and concentrated via solvent evaporation. Samples were reconstituted in one-tenth volume assay buffer, and cGAMP levels were quantified using the DetectX® 2′,3′-cGAMP STING-Based FRET assay (Arbor Assays). For each experimental condition, cGAMP secretion assays were performed in triplicate and data represents a minimum of three biological replicate experiments.

Confocal laser scanning microscopy. Adult primary human gastric epithelial cells were grown on collagen-coated 12 mm glass coverslips (#1.5, 170 μm thickness) overnight prior to challenge by WT or cagX H. pylori at a MOI of 50. As a control, a subset of coverslips was mock infected. After 6 h, coverslips were washed in sterile PBS three times, followed by fixation in 4% paraformaldehyde in PBS for 20 min at room temperature. Coverslips were washed in PBS and cells were permeabilized in confocal blocking buffer (3% bovine serum albumin, 0.1% TritonX-100, 1% saponin in sterile PBS) for 1 h at room temperature. For immunostaining, coverslips were stained with anti-STING monoclonal antibody (Life Technologies, 1:100) in confocal blocking buffer overnight at 4 °C. Coverslips were rigorously washed three times in PBST to remove unbound antibody and were subsequently incubated in AlexaFluor 488-conjugated secondary antibody (Life Technologies, 1:1000) in blocking buffer for 1 h at room temperature. For visualization of the nuclei and actin, samples were stained with stained with 4′,6-diamidino-2-phenylindole (DAPI) and AlexaFluor 594 phalloidin for 1 h at room temperature. Coverslips were washed in PBS and were mounted with ProLong Gold antifade (Life Technologies). Fluorescence images were captured using a 60× silicon immersion objective on an Olympus FV3000 confocal laser scanning microscope and images were acquired and processed using cell Sens software (Olympus). Quantification of STING particle size and number was performed using Fiji software (ImageJ) with automated thresholding and subsequent particle analysis of segmented images for mock infected (7 fields of view, n=90 cells); WT infected (11 fields of view, n=82 cells); and cagX infected (9 fields of view, n=59 cells) gastric epithelial cells. To normalize across imaging conditions, average particle sizes were calculated by multiplying the average pixel area by the pixel resolution for each field of view.

RNA isolation and gene expression analyses. Primary gastric epithelial cells were mock infected (n=3 biological replicates) or co-cultured with WT H. pylori 26695 (n=6 biological replicates) or the corresponding cagX isogenic mutant (n=3 biological replicates) for 6 h. Cells were washed three times with sterile PBS and total RNA was isolated using the Direct-Zol Miniprep Plus kit (Zymo Research) following the manufacturer's protocol. Total RNA was stringently digested with Turbo DNase I (Invitrogen) to remove contaminant DNA. To determine inflammatory gene expression changes in response to H. pylori infection, DNA-free RNA (100 ng per sample) was analyzed on the nCounter Sprint Profiler (NanoString Technologies) using the nCounter Human Host Response Panel (NanoString Technologies), which simultaneously quantifies transcripts for 773 immune-related genes and 12 internal reference genes. Differences in gene expression between experimental groups was calculated using the ROSALIND Platform for nCounter Analysis (rosalind.onramp.bio). Raw data (RCC files) were normalized to internal reference genes the nSolver 4.0 software integrated within the ROSALIND platform (ROSALIND, Inc.). Gene normalization was performed using housekeeping probes selected based on the geNorm algorithm as implemented in the Bioconductor package NormqPCR. Differentially expressed genes in H. pylori challenged cells were determined using the Benjamini-Hochberg P value adjustment method of estimating false discovery rates (FDR), with significance set at p<0.05. Read distribution percentages, violin plots, identity heatmaps, and sample multidimensional scaling (MDS) plots were generated within ROSALIND during sample QC. Read normalization, gene expression fold changes, and the associated P values were calculated using criteria provided by NanoString. Pathway enrichment analysis was performed within ROSALIND using the REACTOME database, and gene term enrichment was calculated using a hypergeometric distribution algorithm in reference to the background set of genes in the panel with significance set at p<0.01 and greater than ±1.8-fold gene expression enrichment. Volcano plots and heat maps were generated in GraphPad Prism using normalized gene expression data exported from ROSALIND. Volcano plots were constructed by plotting the log₂ of the normalized fold change versus the −log₁₀ of the adjusted P value for each gene. The dashed P_{adj} lines demarcate genes meeting the threshold for significance (P_{adj}<0.01 and P_{adj}<0.05) after correction with the Benjamini-Hochberg procedure for controlling FDR.

Gastric epithelial cell supernatant transfer assay. Primary gastric epithelial cells seeded into 24-well gelatin-coated dishes were mock infected or were co-cultured with WT H. pylori or the cagX isogenic mutant at a MOI of 100 for 6 h or 12 h. Cell culture supernatants were harvested and treated with 100 g/ml gentamicin for 1 h at 37 °C to eliminate viable bacteria. Supernatants were subsequently spun for 10 min at 10,000×g to remove bacteria and gastric cells, and cleared supernatants were stored at −20 °C. For TLR9 stimulation studies, HEK293-hTLR9 cells were seeded into 96-well plates at a density of approximately 2×10⁴ cells per well in a volume of 100 μl DMEM per well. Primary gastric epithelial cell supernatants were added to hTLR9 reporter cells at an equal volume and incubated for 24 h at 37 °C in 5% CO₂. TLR9 activation was determined via SEAP levels determined by QuantiBlue™ (Invivogen) using a microplate reader (BioTek Synergy HI) to record the absorbance at 650 nm. The concentration of cell-free DNA in processed supernatants was determined by high sensitivity dsDNA Qubit assay (Life Technologies).

For enzyme treatments of gastric epithelial cell supernatants, pre-cleared supernatants obtained from 6 h H. pylori challenged gastric epithelial cells were either treated by (i) the addition of 10 units Turbo DNase (Life Technologies) and incubation at 37 °C for 30 min, (ii) heating the supernatant to 70 °C for 15 min, or (iii) heating the supernatant to 70 °C for 15 min followed by cooling to room temperature and subsequent DNase treatment as described in (i). Treated supernatants were added to HEK293-hTLR9 reporter cells as described for supernatant transfer assays, and TLR9 stimulation was assessed by QuantiBlue™ after 24 h incubation. TLR9 activation is expressed as the fold change over mock treated HEK293-hTLR9 cells.

Colony immunoblotting. To assess bacterial cell surface localization of cag T4SS effectors⁵⁰, H. pylori strains were grown on blood agar plates for 24 h before harvesting and normalization to OD₆₀₀ of 1. Bacteria were pelleted by centrifugation and resuspended in 50 μL Brucella broth to reach an OD₆₀₀ of 20. Concentrated suspensions were spotted onto fresh blood agar plates (5 μL) and incubated at 37 °C, 5% CO₂. After 24 h, a nitrocellulose membrane was placed on top of the colonies for 5 min at room temperature. The membrane was washed twice in TBST (tris-buffered saline, 0.05% Tween 20) and blocked in 5% milk in TBST. Surface exposure of bacterial effectors was assessed by immunoblotting using anti-CagA (α-CagA, Santa Cruz Biotechnology) or anti-dsDNA (α-dsDNA, EMD Millipore). Immunoblotting with anti-H. pylori antibody (a gift from Dr. Tim Cover) was performed as a positive control.

Live cell bacterial immunocapture. H. pylori was grown for 24 h on blood agar plates prior to collection and normalization to an OD₆₀₀ of 0.5 in Brucella broth supplemented with 6% FBS. Normalized bacteria were incubated with 250 μg/mL anti-dsDNA monoclonal antibody (α-dsDNA, EMD Millipore) or an equivalent amount of IgG isotype control antibody (Thermo) for 1 h in the presence of 5% CO₂ at 37 °C. After incubation, an aliquot was obtained for total colony forming unit (CFU) enumeration by serial dilution plating. The remaining sample was incubated with Protein G Dynabeads (Invitrogen) for 10 minutes at RT with constant rotation. Beads were isolated by magnetic separation and washed twice with 5 bead volumes of sterile PBS. Beads were then resuspended in 100 μL sterile PBS followed by serial dilution and plating for CFU enumeration. For DNase treated samples, H. pylori pellets were resuspended in PBS and normalized to an OD₆₀₀ of 1 prior to incubation with 5 μL DNase buffer and 1 unit Turbo DNase I (Life Technologies). After 15 min incubation, an equal volume of Brucella broth supplemented with 6% FBS was added to each well to reach an OD₆₀₀ of 0.5, and bacterial immunocapture was performed. Levels of H. pylori displaying dsDNA on the bacterial cell surface were calculated as a percentage of total bacteria in each sample and normalized to levels of bacteria isolated in IgG isotype control purifications.

Erythromycin susceptibility assays. H. pylori strains cultured for 24 h on blood agar plates were collected, normalized to an OD₆₀₀ of 1, and an equivalent volume of normalized cultures were inoculated onto fresh blood agar by sterile bead plating. Erythromycin-impregnated discs (15 μg) were placed onto dried plates, and the diameter of the zone of inhibition was determined after 24 h incubation at 37 °C and 5% CO₂. Susceptibility assays were performed a minimum of three times for each H. pylori strain.

Extracellular vesicle (EV) purification. Primary gastric epithelial cells were challenged by H. pylori or were mock infected for 6 h. Supernatants were pre-cleared by serial centrifugations (10,000×g) at 4 °C. EV-containing supernatants (2 ml per biological replicate) were subsequently magnetically labeled for 1 h at room temperature by CD9, CD63, and CD81 antibodies (Human Exosome Isolation Kit, Pan, Miltenyi Biotec) followed by EV isolation via magnetic separation and elution. Immunoaffinity purified exosomes were subjected to qPCR analysis probing for H. pylori genomic DNA (hp1421 locus, 290 bp fragment) and mtDNA (coxII, 444 bp fragment) by Fast SYBR Green chemistry (Life Technologies) on a Viia7 platform (Thermo). H. pylori DNA enrichment within purified EVs was determined by quantifying the ratio of hp1421 C_{T} values to coxII C_{T} values for EVs obtained from WT and cagX-infected gastric cells. EVs purified from mock infected gastric epithelia contained levels of mtDNA similar to EVs derived from H. pylori-challenged primary cells.

Immunoprecipitation of cGAS-DNA complexes. 293T cells were grown in 6-well plates for 24-30 hours to achieve approximately 90% confluency. Cells were transfected with pUNO 1-hcGAS-HA3X (240 ng DNA/well) complexed to Lipofectamine 2000. After 12-16 h, approximately 7.2×10⁶ transfected 293T cells were challenged by exponentially growing WT or cagX H. pylori at a MOI of 100. As a control, an equivalent number of transfected 293T cells were mock infected. After 6 h of infection, 293T cells were rinsed with PBS and DNA-protein complexes were cross-linked by 1% paraformaldehyde for 15 min at room temperature. Cross-linking reactions were quenched by the addition of 250 mM glycine, and cells were collected by mechanical detachment and centrifugation at 4000 rpm for 10 min. Cell pellets were washed once with sterile PBS, followed by re-suspension in pre-chilled lysis buffer (5 mM EDTA, 1% NP-40, and 1× protease inhibitor cocktail in PBS) and sonication at 5% amplitude (10 Sec ON, 10 Sec OFF, 4-6 cycles) to generate cleared lysates. Sonicated cell extracts were centrifuged at 14,000 rpm for 30 min at 4 °C. The recovered cell-free supernatant was incubated with 4 μg of anti-HA monoclonal antibodies (clone 12CA5) overnight at 4 °C with continuous end-over-end rotation. The following day, 50 μl of Protein G Dynabeads (Life Technologies) pre-blocked in PBS containing 1% BSA were incubated with immunopurification samples for 90-120 min at 4 °C with continuous end-over-end rotation. Dynabeads were collected by magnetic isolation, washed twice with 1× cell lysis buffer, followed by one was in high salt wash buffer (cell lysis buffer+300 mM NaCl). Magnetic beads were re-suspended in 100 μl of 1% SDS +0.1 M sodium bicarbonate buffer and de-crosslinked by incubation at 60 °C overnight. Purification of cGAS-HA3x was confirmed in the eluted fractions by immunoblot analysis. To purify DNA complexed with cGAS-HA3x, de-crosslinked fractions were treated with 20 μg of Proteinase K (Sigma-Aldrich) at 60 °C for 1-2 h, followed by DNA isolation via the ChIP DNA Clean and concentrator kit (Zymo Research) according to the recommended protocol. Eluted DNA was quantified using the Qubit high sensitivity dsDNA assay (Life Technologies).

cGAS ‘ChIP-seq’ library preparation and sequencing. ChIP samples were quantified using Qubit 2.0 Fluorometer (Life Technologies) the DNA integrity was analyzed with 4200 TapeStation (Agilent Technologies). cGAS ‘ChIP-seq’ library preparation and sequencing reactions were conducted at GENEWIZ, Inc. (South Plainfield, NJ, USA). NEB NextUltra DNA Library Preparation kit was used following the manufacturer's recommendations (Illumina). Briefly, DNA eluted from cGAS immunopurifications was end repaired and adapters were ligated after adenylation of the 3′ ends. Adapter-ligated DNA was size selected, followed by clean up, and limited cycle PCR enrichment. The cGAS ‘ChIP’ library was validated using Agilent TapeStation and quantified using Qubit 2.0 Fluorometer as well as RT-PCR (Applied Biosystems). Sequencing libraries were multiplexed and clustered on two lanes of a flowcell.

After clustering, the flowcell was loaded on the Illumina HiSeq instrument according to manufacturer's protocol (Illumina). Sequencing was performed using a 2×150 paired end (PE) configuration. Image analysis and base calling were conducted by the HiSeq Control Software (HCS). Raw sequence data generated from Illumina HiSeq was converted into fastq files and de-multiplexed using Illumina's bcl2fastq 2.17 software. One mismatch was allowed for index sequence identification. Sequence reads were processed to remove adapter sequences and nucleotides with poor quality at both 5′ and 3′ ends using CLC Genomics workbench. Sequence reads below 15 bases were discarded. Trimmed data was aligned to both human (Homo sapiens reference genome hg38) and H. pylori 26695 (reference genome NC_000915) reference genomes. Only specific alignment was allowed during mapping. To detect peaks that were differentially present in cGAS purifications obtained from WT-infected cells versus cagX-infected cells, reads were normalized to mock infected control preparations, and peak calling was performed using the Transcription Factor model within CLC Genomics workbench with p<0.01 considered significant.

Nanoluciferase (NanoLuc) bioluminescence assays. AGS or 293T cells were cultured overnight to reach approximately 70% confluence, and were challenged by WT or cagX H. pylori [NanoLuc] (26695) or WT or cagE H. pylori [NanoLuc] (G27) at a MOI of 50 for 24 h. Cell culture supernatants were removed and monolayers were washed in sterile PBS to remove non-adherent cells. Monolayers were lysed in 1% NP-40, and 50 μl of cell lysate was transferred to a white walled 96-well plate. To measure nanoluciferase bioluminescence, 20 μl Nano-Glo Luciferase substrate assay buffer containing furimazine (Promega) prepared according to the manufacturer protocol was added to each well. Luciferase activity was immediately assessed using a BioTek Synergy H1 plate reader with luminesce acquisition settings set as recommended by the manufacturer, with the exception of the gain which was adjusted to 230. To determine the level of background NanoLuc activity produced by H. pylori, 20 μl of overnight bacterial cultures were directly lysed in Nano-Glo Luciferase substrate assay buffer containing furimazine, and luciferase values were immediately obtained via plate reader using the same parameters as for eukaryotic cells. Background luminescence produced by H. pylori was determined by normalizing luciferase values by the corresponding culture OD₆₀₀ and is expressed as the fold change in luminescence over values obtained for H. pylori cultures that do not harbor the nanoluciferase expression construct. Bioluminescence of H. pylori-challenged wells is expressed as the fold change over mock infected wells. For each eukaryotic cell line, a minimum of four biological replicate experiments were performed in 24-well plate technical replicate format.

Live cell fluorescence microscopy analysis of LifeAct-mScarlet. H. pylori harboring LifeAct-mScarlet constructs were grown to exponential phase in broth culture and AGS or 293T monolayers were inoculated at a MOI of 50 overnight at 37 °C in 5% CO₂. Monolayers were washed in sterile PBS to remove non-adherent bacteria, and monolayers were imaged via live cell, phase contrast epi-fluorescence microscopy on a Nikon Ti Eclipse equipped with a 594ₑₘ filter and a Plan Apo VC 20X/0.75 NA air objective. Fluorescent images were superimposed on the corresponding phase contrast image of the same field of view. Images were processed for equivalent contrast, brightness, and magnification using the OMERO platform (Allan et al., 2012).

Flow cytometry analysis. AGS or 293T cells were cultured overnight to reach approximately 70% confluence, and were challenged by WT or cagX H. pylori [LifeAct-mScarlet] at a MOI of 50 for 24 h. Cell culture supernatants were removed and monolayers were washed in sterile PBS to remove non-adherent cells. Cells were trypsinized (AGS) or mechanically detached (293T) from tissue culture flasks, washed once in PBS via centrifugation and pelleting, and re-suspended in PBS at approximately 1×10⁶ cells/ml. Samples were analyzed on an Attune™ NxT Flow Cytometer (Thermo). Forward scatter-height (FSC-H) and sideward scatter-height (SSC-H) profiles were used in gating strategies to select for single cells, and positive mScarlet fluorescence gates were determined by analyzing 293T cells that had been transfected with pLifeAct_mScarlet-i_N1 (Bindels et al., 2017).

IL-8 quantitation. Interleukin-8 (IL-8) secretion was monitored by human CXCL8 ELISA (R&D Systems) as previously described (Shaffer et al., 2011; Varga et al., 2021). Briefly, AGS cells were plated in 24-well dishes and were cultured overnight prior to infection with H. pylori at a MOI of 100 for 4.5 h. Supernatants were collected and stored at −20 °C until analysis by ELISA. A minimum of three biological replicate experiments were performed in triplicate for all strains, and IL-8 secretion is expressed as a percent of IL-8 levels induced by WT H. pylori for each replicate experiment.

‘Transfer DNA’ immunoprecipitation. To assess whether processed chromosomal DNA fragments physically associate with the cag T4SS outer membrane complex, H. pylori grown for 24 h on blood agar were harvested in 2 ml PBS, and 50 μL of the cell suspension were removed to serve as the ‘input’. The remainder of the collected cells were pelleted by centrifugation and were washed once in PBS. Cell pellets were re-suspended in 500 μL PBS, and protein-DNA complexes were cross-linked by the addition of 500 μL 1% paraformaldehyde for 10 min at room temperature, followed by quenching with 1 ml 250 mM glycine. Cross-linked cells were pelleted and washed once with PBS. Cell pellets were re-suspended in 1 mL lysis buffer (5 mM EDTA, 1% NP-40 in PBS) supplemented with 2× cOmplete™ inhibitor (Roche) and were sonicated until the lysate became turbid. Cell lysates were treated with 2 units of Turbo DNase (Life Technologies) in 10 mM MgCl₂ for 30 min at room temperature. To solubilize membranes, 0.2% SDS and 0.2% sodium deoxycholic acid (final concentrations) were added to cell lysates, and samples and rotated end-over-end for 1-2 h. Supernatants were separated from insoluble cell debris by centrifugation at 14,000 rpm for 30 minutes at 4 °C. In a separate tube, 4 μl polyclonal anti-CagY antisera (a kind gift from Dr. Tim Cover) was added to 800 μl lysis buffer containing 10 mM MgCl₂, 2 units Turbo DNase, and 25 μl Protein G Dynabeads, and was incubated for 10 min at room temperature. Cleared supernatants were added directly to CagY antibody solutions and were incubated for an additional 2-4 h with continuous end-over-end rotation. Beads were isolated by magnetic separation and were washed twice in PBS supplemented with 10 mM MgCl 2 and 1 unit Turbo DNase, followed by two washes in high salt buffer (lysis buffer containing 400 mM NaCl), and a final wash in PBS. Protein-DNA complexes were eluted and de-cross-linked in 100 μl 1% SDS in 0.1 M NaHCO₃ at 65 °C overnight. The following day, proteins were digested using Proteinase K (10 μg) at 65 °C for 30 minutes. DNA was precipitated by 100% ethanol in 0.3 M sodium acetate (1:3 v/v) at −20 °C. Precipitated DNA was pelleted by centrifugation at 14,000 rpm for 30 minutes at 4 °C, washed once with 70% ethanol, and re-hydrated in 20 μl ultrapure water. To serve as a control, the initial ‘input’ cell pellet was re-suspended in 100 μl of 50 μM NaOH, and DNA was liberated by incubating at 95 °C 30 min, followed by pH neutralization by the addition of 10 μl of 1M Tris, pH 8. To assess chromosomal DNA association with immunopurified CagY complexes, 1 μl of ‘input’ and ‘IP’ DNA samples were used as the template in standard PCR assays targeting a 795 bp chromosomal DNA amplicon. Quantitation of DNA amplification from ‘input’ and ‘IP’ samples was conducted by densitometry analysis and amplification efficiency of ‘IP’ samples was calculated as a percent of the corresponding ‘input’ sample amplification for each biological replicate experiment. Transfer DNA immunopurification assays were performed a minimum of four times per strain.

Recombinant protein purification. Proteins of interest were cloned from H. pylori 26695 into pMAL-c6T (New England Biolabs) to permit protein fusion overexpression and purification via affinity chromatography as previously described (Grove et al., 2013). Briefly, MBP fusions were expressed in E. coli BL21 (NEB) by induction via 0.5 mM isopropyl β-D-1-thiogalactopyranoside for 3 h at 37 °C. Bacteria were harvested by centrifugation, and cells were lysed by sonication in purification buffer (20 mM Tris pH 7.5, 500 mM NaCl, 1 mM EDTA, 5 mM BME, 1× protease inhibitor cocktail) followed by centrifugation at 14,000 rpm for 30 min at 4 °C. Soluble MBP fusions were incubated with pre-equilibrated amylose resin (NEB) for 1 h at 4 °C and columns were washed to remove unbound protein with 5 column volumes of purification buffer. MBP fusions were eluted in elution buffer (10 mM maltose, 20 mM Tris pH 7.5, 100 mM NaCl, 1 mM EDTA, 5 mM BME). Protein size and purity was determined by SDS-PAGE analysis, and protein concentration was determined by Bradford assay (Bio-Rad). Purified MBP fusions were stored in elution buffer supplemented with 10% glycerol at −80 °C for DNA binding and cleavage assays.

DNA binding and cleavage assays. 5′ or 3′ Cy5-labelled chemically synthesized oligonucleotides containing the tfs3 oriT-like recognition sites (Grove et al., 2013) were incubated with varying concentrations of MBP fusion proteins ranging from 0 to 4 μM. For electrophoretic mobility shift assays, DNA binding reactions were assembled in 15 μl total volumes with 10 nM substrate in reaction buffer (50 mM Tris-HCl pH 8, 50 mM KCl, 0.5 mM MgCl2, 1 mM DTT, 0.1 μg/μl BSA) for 30 min at 4 °C. Free DNA and nucleoprotein complexes were resolved using native PAGE [8-10% Acrylamide (29:1)] run in 1× TBE at 70V for 2-3 h. For DNA cleavage assays, 20 nM DNA substrate and fusion proteins were assayed in 10 μl reaction in cleavage buffer (50 mM Tris-HCl pH 8, 50 mM KC1, 5 mM MgCl₂, 1 mM DTT, 0.1μg/μl BSA) for 90 min at 37 °C. Proteinase K treatment was performed in presence of 0.1% SDS followed by boiling in formamide at 95 °C for 15 min, and the reaction products were resolved by denaturing Urea-PAGE (15%) in 1× TBE. For DNA nicking and linearization assays, 30 nM of supercoiled pJet1.2 containing a single copy of the tfs3 oriT-like region was incubated with MBP fusion proteins in reaction buffer (50 mM Tris pH 8, 50 mM KCl, 5 mM MgCl₂, 1 mM DTT, 0.1 μg/ml BSA) for 30 min at 37 °C. Reactions were stopped by the addition of 50 mM EDTA, Proteinase K (10 μg), and 0.1 % SDS in 0.1% bromphenol blue loading dye for 15 min at 70 °C. Reaction products were resolved and visualized via electrophoresis in a 1% agarose gel in 1× TAE buffer.

DNA binding electromobility shift assays (EMSA). 5′ Cy5-labeled chemically synthesized oligonucleotides containing the tfs3 oriT-like recognition sites⁸³ were incubated with varying concentrations of MBP fusion proteins ranging from 0 μM to 8 μM. DNA binding reactions were assembled in 15 μl total volume containing 0.01 μM DNA substrate in reaction buffer (50 mM Tris-HCl pH 8, 50 mM KCl, 0.5 mM MgCl₂, 1 mM DTT, 0.1 μg/μl BSA) for 30 min at 4 °C. Free DNA and nucleoprotein complexes were resolved using native PAGE (10%) run in 1× TBE at 70V for 2-3h. For competition assays, unlabeled competitor oligonucleotides (0.01 μM to 0.4 μM) were added to the reaction mixture containing Cy5-labeled oligonucleotides (0.01 μM) and MBP-CagX (0.75 μM). After incubation on ice for 30 min, reaction mixtures were resolved using native PAGE (10%) run in 1× TBE at 70V for 2-3h at 4 °C. Quantification of free DNA was performed using ImageLab software (Bio-Rad).

Protein structure modeling. CagC predicted structure was determined using AlphaFold2colab^{54,55}. Protein sequence alignments were performed using MegAlign Pro (DNAstar). Superimposed images of protein structures were generated using ChimeraX Matchmaker⁸⁴. Structural analyses of the H. pylori cag T4SS PRC were conducted on PDB 6X6J. The electrostatic surfaces of CagX, TraK, and VirB9 were generated as coulombic potential maps in ChimeraX.

Bacterial adenylate cyclase two-hybrid (BACTH) assays. To probe direct interactions between proteins, we employed a bacterial two-hybrid system in which the full-length Hp0996 bait was fused to the T18 N-terminus (pUT18 vector) as previously described (Damke et al., 2022; Karimova et al., 2017). Plasmids encoding various prey proteins were cloned as N-terminal fusions to the T25 subunit (pKNT25 vector). Recombinant plasmids were verified by whole plasmid DNA sequencing. Bait and prey plasmids were co-transformed into E. coli strain BTH101 and transformants were selected on dual antibiotic (100 μg/mL ampicillin and 50 μg/mL kanamycin) LB plates at 30 °C. Empty T18 and T25 vectors were co-transformed to serve as a negative control while T18-Zip/T25-Zip (Karimova et al., 2017) and T18-DprA/T25-DprA (Mortier-Barriere et al., 2007) were co-transformed into BTH101 to serve as positive controls. Individual colonies were selected, grown overnight in at 30 °C in LB supplemented with both selective antibiotics and 0.5 mM IPTG, and were spotted onto dual antibiotic LB plates supplemented with 0.5 mM IPTG and 40 μg/mL X-gal followed by incubation at 30 °C. Plates were stored at RT to monitor colony color (blue indicating positive protein-protein interactions). To confirm protein-protein interactions, overnight cultures were inoculated into M63 minimal media ((NH₄)₂SO₄ (2 g/L), KH₂PO₄ (13.6 g/L), thiamine B1 (1 mg/L), 1 mM MgSO₄, FeSO₄.7H₂O (5 mg/L), pH 7) supplemented with 0.4% maltose, 0.5 mM IPTG and selective antibiotics (50 μg/mL ampicillin and 30 μg/mL kanamycin), and were grown under aerobic conditions at 30 °C for 72 h (positive protein-protein interactions indicated by growth in M63-maltose minimal medium). Bacterial growth was assayed by monitoring the optical density at 600 nm (Biotek Synergy H1) and compared to positive (Zip-Zip) and negative (empty vector) control strains. Results represent the fold change in culture OD600 over the corresponding negative control cultures from a minimum of at least three independent biological replicate experiments.

Immunoblotting. To detect bacterial protein expression, normalized samples were lysed in reducing 2× SDS buffer (Bio-Rad), resolved by SDS-PAGE (10%), transferred to a nitrocellulose membrane, and immunoblotted using rabbit polyclonal antisera raised against CagC peptides⁶⁹ or recombinant CagX (CagC and CagX antisera were a gift from Dr. Tim Cover) as previously described⁴⁸. To confirm equal sample loading, membranes were immunoblotted using H. pylori outer membrane protein monoclonal antibody (α-OMP, Santa Cruz) or anti-H. pylori polyclonal antisera raised against H. pylori whole cell lysate (a gift from Dr. Tim Cover). Secondary detection by horse radish peroxidase-conjugated anti-rabbit IgG or anti-mouse IgG was achieved using SuperSignal West Pico chemiluminescent substrate (Thermo).

Statistical analyses. Data are expressed as mean values ±standard error of the mean, which were calculated from a minimum of three biological replicate experiments. In all graphs, each data point represents an individual measurement, lines represent the mean, and error bars represent the standard error of the mean. Statistical analyses were performed using GraphPad Prism 9 software, with differences of p<0.05 considered statistically significant.

### Results

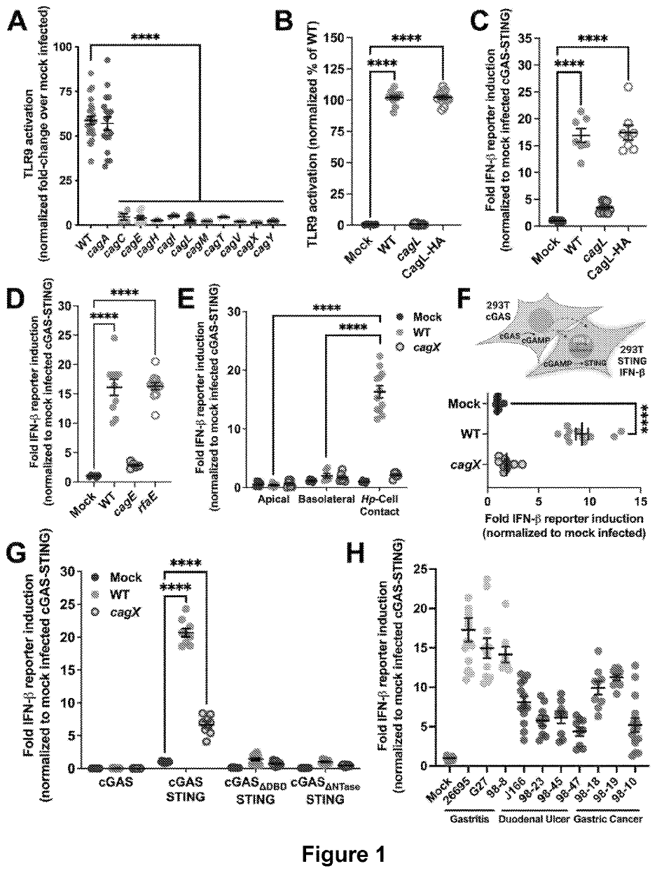

H. pylori provokes multiple DNA surveillance systems in a cag T4SS-dependent manner. Previous studies demonstrate the capacity of H. pylori to stimulate DNA-sensing pattern recognition receptors, including TLR9 (Rad et al., 2009; Varga et al., 2016b), raising the hypothesis that microbial nucleic acids are actively translocated into host cells. In agreement with previous reports (Rad et al., 2009; Varga et al., 2016b), H. pylori challenge of HEK293 reporter cell lines stably transfected with TLR9 demonstrated that cag T4SS activity is required for TLR9 stimulation (FIG. 1A,B). Consistent with the observation that CagA is not translocated into HEK293 cells (Kumar Pachathundikandi et al., 2011; Varga et al., 2016b), disruption of cagA did not diminish levels of H. pylori-induced TLR9 activation (FIG. 1A). To confirm that a functional cag T4SS is required to activate TLR9, we co-cultured TLR9 reporter cells with a cagL isogenic mutant or the corresponding genetically complemented strain. Whereas inactivation of cagL abrogated TLR9 activation, complementation in a heterologous chromosomal locus rescued TLR9 stimulation to levels indistinguishable from the parental WT strain (FIG. 1B). In concert with previous investigations, these data demonstrate that cag T4SS activity, but not CagA delivery, is required for robust TLR9 signaling.

In addition to endosomal DNA pattern recognition receptors such as TLR9, gastric epithelial cells harbor cytosolic DNA surveillance proteins including the nucleotidyltransferase cyclic GMP-AMP (cGAMP) synthase (Cai et al., 2014; Diner et al., 2013; Sun et al., 2013; Wu et al., 2013). To test whether H. pylori DNA is trafficked into the host cell cytoplasm to activate cytosolic DNA surveillance sensors, we challenged 293T cells transfected with constructs to enable cGAS and STING expression and quantified levels of IFN-β promoter-driven luciferase produced in response to H. pylori. Compared to mock infected cells, WT H. pylori stimulated high levels of cGAS-STING signaling at 18 h post-infection. Similar to TLR9 activation assays, the cagL mutant was unable to stimulate robust cGAS-STING signaling, a phenotype that was restored by genetic complementation in a secondary chromosomal locus (FIG. 1C). In contrast to HEK293-hTLR9 cells, moderate levels of translocated CagA were detected in 293T cells co-cultured with WT H. pylori but not in corresponding cagE-challenged monolayers (FIG. S1A). Although H. pylori has the capacity to deliver CagA into 293T cells, disruption of cagA did not significantly alter levels of cag T4SS-dependent IFN-β promoter activity in cGAS-STING reporter cells (FIG. S1B). To exclude the possibility that cGAS-STING activation results from bacterial endocytosis or increased bacterial interaction with host cell surfaces, we performed gentamicin protection assays. In contrast to marked differences in cGAS-STING activation elicited by WT and cagX, equivalent levels of adherent and intracellular bacteria were recovered from 293T co-cultures, confirming that cGAS-STING signaling is not an artifact of non-specific bacterial internalization or spontaneous bacterial lysis (FIG. S1C).

Previous studies demonstrate that in addition to detecting invading microbial threats, cGAS senses and responds to DNA damage and genomic instability (Cai et al., 2014; Ke et al., 2022). H. pylori cag T4SS activity is directly linked to nuclear double-stranded DNA (dsDNA) breaks introduced in response to ALPK1/TIFA signaling stimulated by D-glycero-beta-D-manno-heptose 1,7-bisphosphate (HBP) or ADP-beta-D-manno-heptose (β-ADP-heptose) translocation (Bauer et al., 2020; Gall et al., 2017; Zimmermann et al., 2017). We therefore addressed the possibility that cag T4SS-dependent nuclear DNA damage stimulates cGAS-STING signaling elicited by WT H. pylori. Disruption of rfaE, the enzyme responsible for β-ADP-heptose production (Bauer et al., 2020; Gall et al., 2017; Stein et al., 2017; Zimmermann et al., 2017), did not impact the level of cGAS-STING signaling achieved by H. pylori (FIG. 1D), indicating that ALPK1/TIFA signaling-induced DNA damage does not significantly contribute to H. pylori-driven cGAS activation in vitro. In addition to ALPK1/TIFA-mediated DNA modifications (Bauer et al., 2020; Gall et al., 2017; Stein et al., 2017; Zimmermann et al., 2017), H. pylori has the capacity to induce production of DNA-damaging reactive oxygen species (ROS) by triggering NF-_{κ}B activation and additional mechanisms involving inducible nitric oxide synthase (iNOS) and associated inflammatory enzymes (Bauer et al., 2020; Kidane, 2018). In support of the observation that cag T4SS-induced dsDNA breaks are not a significant cGAS activating factor in the context of H. pylori infection, cGAS-STING activation was achieved by H. pylori co-cultured in the presence of the antioxidant N-acetyl-cysteine at concentrations that abrogate ROS production (Bauer et al., 2020) (FIG. S1D).

Following infection, damaged mitochondria release DNA (mtDNA) and other constituents into the cytosol to act as potent danger-associated molecular patterns (DAMPs) that engage TLR9 (Garcia-Martinez et al., 2016; Oka et al., 2012; Zhang et al., 2010) and cGAS-STING signaling axes to initiate type I IFN production (Rongvaux et al., 2014; West et al., 2015; White et al., 2014). To test the hypothesis that H. pylori cag T4SS activity modulates mitochondrial integrity resulting in the release of mtDNA and activation of cytosolic DNA-sensing PRRs, we assayed cGAS-STING activation in the presence of BAX/BAK macropore inhibitory peptides that prevent permeabilization of the mitochondrial outer membrane and herniation of mtDNA into the cytosol (McArthur et al., 2018; White et al., 2014). H. pylori induced similar levels of cGAS-STING activation in the presence of BAX inhibitory peptide or non-inhibitory peptide control co-cultures (FIG. S1E) suggesting that ruptured mitochondria are not the primary source of cGAS-activating DNA. Likewise, treatment of co-cultures with a mitochondria-targeted antioxidant did not significantly alter levels of cGAS-STING signaling (FIG. S1D), demonstrating that H. pylori-induced mtDNA damage is not a predominant DAMP within the context of cag T4SS-dependent cGAS activation. To exclude the possibility that cGAS-STING activation is dependent upon the import of released H. pylori DNA via host cell mechanisms, we quantified IFN-β promoter activity produced by cGAS-STING reporter cells that were physically separated from H. pylori. In support of the hypothesis that cGAS-STING activation requires direct H. pylori-host cell interaction, cGAS stimulation was achieved by H. pylori that were in direct contact with reporter cells, but not in samples in which H. pylori and reporter cells were physically separated by a 0.4 μM-pore polycarbonate insert (FIG. 1E). Collectively, these studies demonstrate that cGAS stimulation is a consequence of cag T4SS-dependent DNA delivery into the host cell cytosol.

To define the role of cGAS in H. pylori-driven IFN-β signaling, we next determined whether cag T4SS activity could transactivate STING through direct cGAMP transfer via gap junction-mediated diffusion. To assess cGAS signaling in trans, 293T cells transfected with cGAS constructs were co-cultured with 293T cells harboring STING and IFN-β reporter constructs, and co-cultures were challenged by H. pylori for 18 h. High levels of STING signaling were observed in cells challenged by WT H. pylori, but not a cagX mutant, suggesting that cag T4SS-dependent cGAS stimulation generates sufficient cGAMP for migration into bystander cells. To examine the requirement of cGAS protein domains in H. pylori-induced STING signaling, we next monitored IFN-β activation in 293T cells transfected with constructs to express either cGAS alone or cGAS variants in combination with STING. In comparison to cells transfected with only cGAS_{WT}, WT H. pylori stimulated high levels of IFN-β transcription when cGAS_{WT} and STING were co-expressed (FIG. 1G). In contrast, cag T4SS-dependent IFN-β transcription was markedly reduced in 293T cells transfected with STING and cGAS variants lacking the DNA-binding domain (cGAS_{ΔDBD}) or harboring point mutations within nucleotidyltransferase catalytic residues that abolish cGAMP production (cGAS_{ΔNTase}). Collectively, these results demonstrate that cGAS senses and responds to cag T4SS activity.

A previous report suggests that decreased STING signaling is associated with adverse outcomes in gastric cancer patients (Song et al., 2017). We therefore monitored cGAS-STING signaling induced by several H. pylori clinical isolates obtained from patients with gastric diseases of varying severity. In contrast to strains isolated from patients exhibiting gastritis, H. pylori isolated from individuals with duodenal ulcers or gastric cancer elicited lower levels of cGAS-STING signaling (FIG. 1H), suggesting that H. pylori may modulate the capacity to induce cag T4SS-dependent STING activation during chronic stomach colonization.

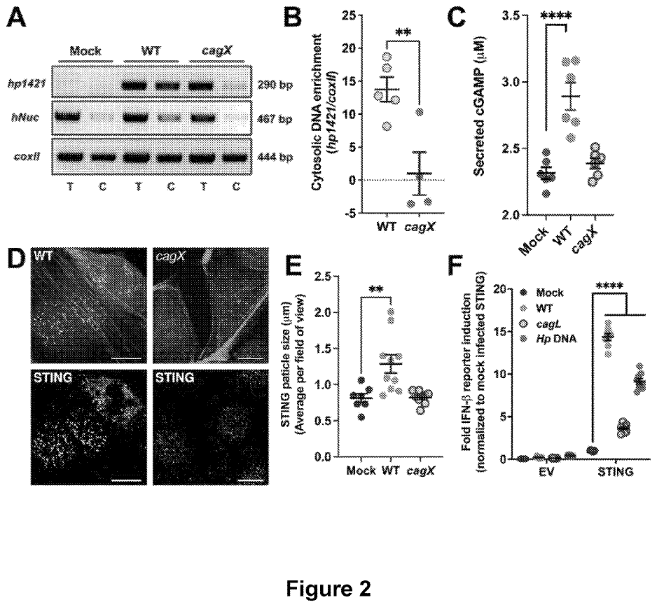

Microbial DNA is delivered to the gastric epithelial cell cytoplasm via cag T4SS activity. We next assessed the consequence of H. pylori trans-kingdom DNA conjugation within the context of biologically-relevant interactions with gastric epithelial cells. To monitor DNA injection into gastric epithelial cells, AGS cells were challenged by either WT or the cagX isogenic mutant at a MOI of 50. After 6 h, gastric epithelial cell co-cultures were treated with DNasel to remove extracellular DNA and eukaryotic cells were fractionated using digitonin to selectively permeabilize the plasma membrane, leaving the nuclear envelope and bacterial cells intact. PCR analysis of fractionated infected AGS cells revealed the presence of H. pylori DNA in cytoplasmic fractions (FIG. 2A) with significantly more bacterial DNA present in cytosolic fractions obtained from WT-challenged cells. When comparing the ratio of cytoplasmic bacterial DNA to cytoplasmic-localized mitochondrial DNA by qPCR, significantly more bacterial DNA was present in the cytosol of gastric epithelial cells infected by the WT strain compared to corresponding cells infected by the cagX mutant (FIG. 2B). Levels of mitochondrial DNA did not significantly differ in cytosolic extracts obtained from infected and uninfected cells (FIG. 2A). To exclude the possibility that cytosolic localization of H. pylori DNA resulted from non-specific bacterial lysis or endocytosis, we performed studies to analyze the level of adherent and internalized H. pylori in infected gastric epithelial cells. Using gentamicin-protection assays, we determined that similar levels of adherent and intracellular WT and cagX bacteria were recovered from AGS co-cultures (data not shown). Multiple cancer cell lines, including cell lines derived from gastric adenocarcinoma, continuously export low levels of extracellular cGAMP that serves as a potent immunotransmitter (Carozza et al., 2020). We thus measured levels of extracellular cGAMP secreted by AGS cells in response to H. pylori challenge. Consistent with the hypothesis that cag T4SS-mediated DNA delivery stimulates cytosolic cGAS and the subsequent production of cGAMP, the level of extracellular cGAMP was significantly higher in supernatants obtained from WT-challenged co-cultures compared to mock infected or corresponding cagX-challenged monolayers (FIG. 2C). Collectively, these results suggest that the presence of bacterial DNA in the host cell cytoplasm is a consequence of cag T4SS activity and leads to the production of cGAS-dependent cGAMP.

Although gastric adenocarcinoma cell lines (including AGS and MKN45) produce detectable levels of cGAS and other nucleic acid-sensing PRRs, STING expression is absent (Qiao et al., 2020). To determine whether translocated H. pylori DNA elicits cGAS-STING responses in normal gastric epithelia, we challenged primary adult gastric epithelial cells with H. pylori and monitored the formation of peri-nuclear STING polymers that aggregate in response to cGAMP binding. Compared to mock infected or cagX-challenged cells, WT H. pylori induced the formation of large STING aggregates that could be visualized by confocal microscopy (FIG. 2D). Consistent with activation of cGAS-STING signaling, quantification of the average STING polymer size in H. pylori-gastric cell co-cultures revealed significantly larger STING aggregates in WT-challenged cells (FIG. 2E), suggesting cag T4SS-dependent stimulation of DNA-sensing pathways. In addition to cGAS, epithelial cells harbor other nucleic acid reconnaissance systems that signal through STING. To determine whether H. pylori stimulates additional STING-dependent signaling pathways, we analyzed IFN-β activity in 293T-STING cells. Compared to control 293T cells harboring empty vector, H. pylori stimulated STING signaling in a cag T4SS-dependent manner (FIG. 2F). STING signaling was also elicited by transfection of purified, fragmented H. pylori chromosomal DNA into IFN-β reporter cells (FIG. 2F), suggesting that endogenous 293T cytosolic DNA-sensing pathways respond to H. pylori DNA. Together, these studies demonstrate that H. pylori delivers chromosomal DNA fragments to gastric epithelial cells via cag T4SS mechanisms and reveal that endogenous STING-dependent nucleic acid surveillance systems are activated by translocated H. pylori DNA.

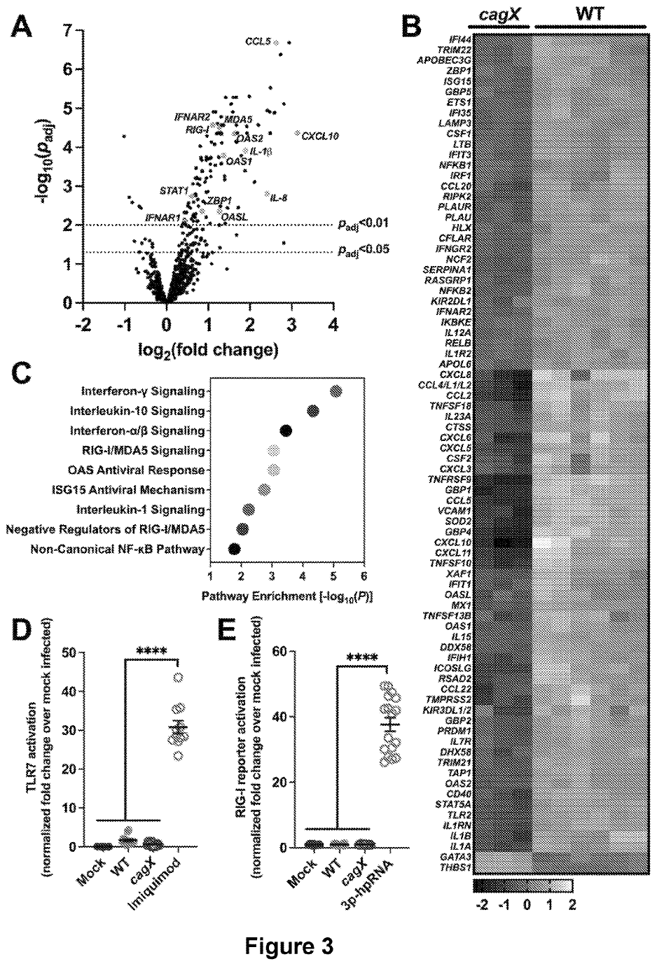

DNA is a specific cag T4SS nucleic acid substrate. We reasoned that in addition to DNA substrates, the cag T4SS may translocate RNA or DNA: RNA hybrids into gastric cells to stimulate STING-dependent signaling. Thus, we sought to characterize the innate inflammatory signature elicited by H. pylori. Primary adult gastric epithelial cells were mock infected or challenged by either WT or the corresponding cagX isogenic mutant prior to isolation of total RNA. To characterize epithelial innate inflammatory responses, gene expression patterns were analyzed at 6 h post-infection using the NanoString Host Response Panel. Compared to mock-infected and cagX-challenged primary gastric epithelial cell co-cultures, cag T4SS activity induced a significant increase in transcripts associated with anti-microbial defense pathways and interferon signaling (FIG. 3A and FIG. S2). Hierarchical clustering performed on genes that were differentially expressed in response to H. pylori challenge revealed distinct clustering of mock infected and cagX-infected co-cultures in comparison to WT challenge (FIG. S2C). Comparison of transcripts differentially produced in response to WT versus cagX H. pylori identified 134 genes that were upregulated via cag T4SS activity, including genes encoding pro-inflammatory chemokines and cytokines (e.g., CXCL10, CXCL8/IL-8, TNF, IL1B, CCL5), IFN-α/β signaling (e.g., ISG15, IRF1, IFI35, STAT1, IFNAR2, SAMHD1), and IFN-β immunoregulatory programs (e.g., IFNGR2, TRIM5, GBP4, VCAM1) (FIG. 3A,B). Consistent with the observed gene expression patterns, pathway analysis of genes induced by cag T4SS activity demonstrated a significant enrichment in transcripts associated with interferon-regulated nucleic acid reconnaissance programs (FIG. 3C). In addition to cag T4SS-dependent regulation of the DNA sensor ZBP1, we observed significantly increased transcript levels of several cytosolic RNA-sensing surveillance systems including DDX58/RIG-I, RIG-I-like Receptor IFIH1/MDA5 (Chow et al., 2015; Dixit and Kagan, 2013), and 2′-5′-oligoadenylate synthetases OAS1 and OAS2 (Schwartz et al., 2020) in WT-infected primary gastric epithelial cells (FIG. 3A,B).

A previous study reported the capacity of dendritic cells to sense and respond to purified H. pylori RNA through RIG-I and TLR 7/8, leading to the production of type I IFN (Rad et al., 2009; Salama et al., 2013). However, whether the cag T4SS actively translocates RNA to stimulate TLR7/8 or RIG-I signaling axes has not been elucidated. We thus sought to determine whether DNA is a specific cag T4SS nucleic acid effector. Challenge of HEK293 reporter cell lines expressing either TLR7 (FIG. 3D) or RIG-1 (FIG. 3E) revealed that in contrast to robust activation of DNA-sensing PRRs, H. pylori was unable to activate either endosomal ssRNA (TLR7) or cytoplasmic ssRNA/dsRNA (RIG-I) sensors. Collectively, these data suggest that chromosomally-derived DNA is a specific cag T4SS substrate, and provide further evidence that cag T4SS-dependent DNA translocation stimulates STING-dependent signaling.

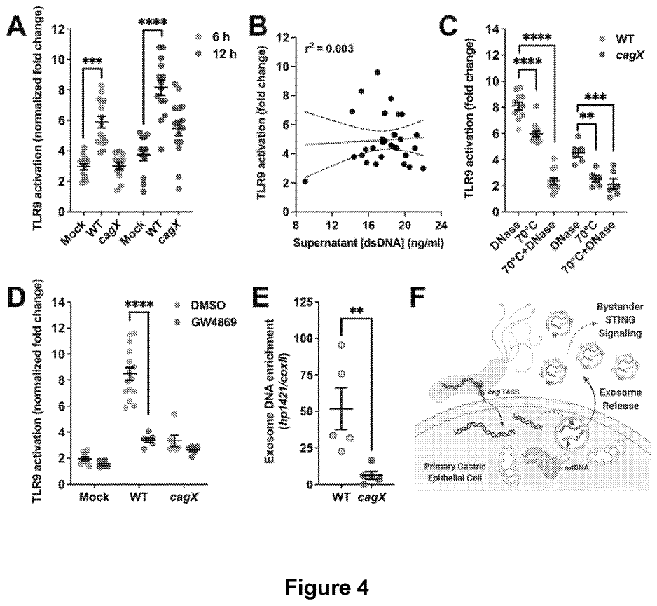

H. pylori cag T4SS activity stimulates DNA immunosurveillance in bystander cells. A previous study reported that foreign intracellular bacterial DNA can be delivered to adjacent cells via extracellular vesicles as a mechanism to amplify IFN signaling (Nandakumar et al., 2019). We therefore investigated whether translocated H. pylori DNA could be sorted into extracellular vesicles to enable paracrine-like DNA signaling by primary gastric epithelial cells. To test the hypothesis that infected gastric epithelial cells release extracellular vesicles containing H. pylori DNA, cell-free supernatants collected from infected primary gastric epithelial donor cells were used to challenge recipient TLR9 reporter cells. Compared to supernatants obtained from either mock infected or cagX-challenged co-cultures, supernatants harvested from WT-challenged gastric cells robustly activated TLR9 signaling (FIG. 4A). The capacity of infected cell supernatants to activate TLR9 was time dependent, as levels of TLR9 activation increased when reporter cells were treated with supernatants obtained from gastric epithelial cells co-cultured with H. pylori for 12 h compared to 6 h (FIG. 4A). To exclude the possibility that TLR9 activation resulted from contaminate DNA arising from cell lysis, we quantified the level of cell-free DNA in donor supernatants. TLR9 activation elicited by gastric cell supernatants was not correlated to the level of donor supernatant cell-free DNA (FIG. 4B), suggesting that the TLR9-stimulating agonist was enclosed within a host cell-derived delivery mechanism.

To confirm the role of translocated DNA in transferrable nucleic acid immunosurveillance in bystander cells, donor gastric cell supernatants were treated with DNase alone or in combination with heat prior to co-culture with TLR9 reporter cells. Whereas DNase treatment had a negligible effect on TLR9 stimulating capacity, heat treatment modesty reduced TLR9 responses, which were further reduced when donor supernatants were treated with both heat and DNase prior to reporter cell challenge (FIG. 4C). These observations led to the hypothesis that translocated microbial DNA packaged within gastric cell-derived extracellular vesicles, such as exosomes, stimulates nucleic acid reconnaissance systems in bystander cells. To test whether exosome biogenesis is required for the delivery of translocated DNA to uninfected cells, we challenged recipient TLR9 reporter cells with donor supernatants obtained from infected primary gastric epithelial cells cultured in the presence or absence of a neutral sphingomyelinase (nSmase2) inhibitor that prevents exosome release. Compared to untreated and mock infected cells, nSmase2 inhibition led to a significant reduction in TLR9 activation levels elicited by WT-challenged donor cell supernatants (FIG. 4D), supporting a role of exosomes in stimulating DNA-sensing pathways in bystander cells. To further investigate whether translocated H. pylori DNA was packaged within exosomes released from infected gastric epithelial cells, we isolated CD9, CD63, and CD81-positive exosomes from cell culture supernatants via immunopurification. Analysis by qPCR revealed that exosomes purified from WT-challenged gastric cell supernatants were significantly enriched in H. pylori DNA compared to corresponding exosomes obtained from cagX-challenged co-cultures (FIG. 4E), suggesting that foreign bacterial DNA is sorted into exosomes for paracrine-like DAMP signal amplification (FIG. 4F).

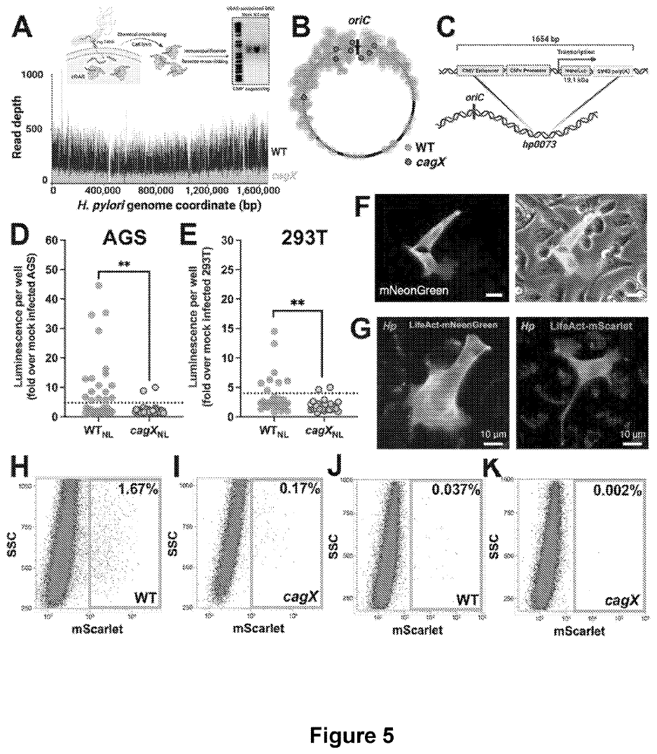

Random chromosomal fragments are delivered to target cells via cag T4SS-dependent mechanisms. To identify whether a specific DNA sequence is excised and transferred to host cells by cag T4SS activity, we performed experiments to purify cGAS-DNA complexes using modified ChIP-seq workflows designed to capture bacterial DNA that physically binds and activates cGAS. 293T-cGAS cells were challenged by H. pylori for 6 h, followed by chemical cross-linking and immunopurification of cGAS. Deep sequencing analysis confirmed that co-purifying H. pylori DNA isolated from WT and cagX-infected cells mapped across the entire H. pylori chromosome, with significantly more bacterial DNA reads associated with cGAS purifications obtained from WT-challenged monolayers (FIG. 5A). Normalized sequencing reads and differential peak calling approaches identified more than three hundred H. pylori chromosomal regions specifically associated with cGAS purified from WT-infected cells compared to eight bacterial DNA peaks associated with corresponding preparations obtained from cagX co-cultures (P=0.01). When enriched peak centers were mapped to the coordinate position across the H. pylori chromosome, differentially enriched peaks heavily clustered around the oriC region, with few peaks mapping to the chromosomal region diametrically opposed to oriC (FIG. 5B), suggesting that DNA translocation is linked to bi-directional DNA replication.

To test the hypothesis that chromosomal regions near oriC are translocated to gastric epithelial cells at a high frequency, we cloned a CMV promoter-driven monomeric nanoluciferase construct into the ureA locus (hp0073) adjacent to oriC (FIG. 5C). Gastric epithelial cells and 293T cells were challenged by WT or cagX H. pylori strains harboring the eukaryotic-optimized nanolucifersase expression construct (WT_{NL} and cagX_{NL}, respectively), and luciferase activity in infected cell lysates was monitored by bioluminescence. Compared to mock infected and cagX_{NL}-infected cells, AGS and 293T cells challenged by WT_{NL} produced high levels of bioluminescence (FIGS. 5D and 5E), indicating that nanoluciferase production by epithelial cells is linked to cag T4SS-dependent DNA translocation.

To confirm that targeted DNA fragments proximal to oriC can be transferred to host cells via cag T4SS mechanisms, we replaced the nanoluciferase gene with a eukaryotic-optimized construct designed to express monomeric mScarlet tethered to the LifeAct N-terminal peptide to enable live cell visualization of F-actin. Microscopy analysis of AGS (FIGS. 5F) and 293T (FIG. 5G) monolayers challenged by WT_{mScarlet} revealed numerous m Scarlet-positive cells compared to corresponding cagX_{mScarlet}-infected co-cultures. Flow cytometry analysis confirmed our observation that significantly more mScarlet-positive epithelial cells were generated when monolayers were challenged by WT_{mScarlet} compared to T4SS-deficient controls (FIG. 5H-K). Together, these data provide direct evidence that targeted chromosomal fragments greater than 1.5 kb in length are excised and delivered to host cells via cag T4SS-dependent mechanisms.

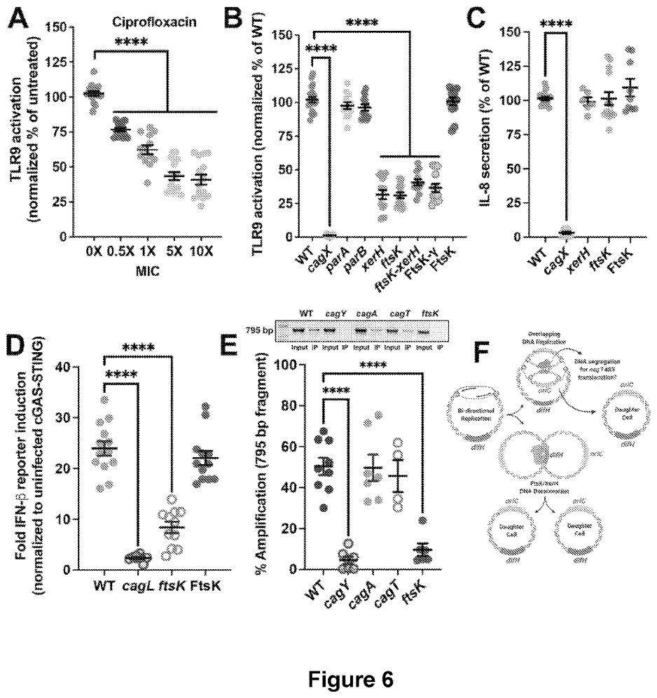

H. pylori trans-kingdom conjugation is mechanistically coupled to chromosomal decatenation. We next sought to understand the mechanism by which effector DNA is coupled to the cag T4SS for transfer to target host cells. The observation that fragments of translocated bacterial DNA map predominantly to the oriC region led to the hypothesis that H. pylori trans-kingdom conjugation is linked to chromosomal replication. In support of this hypothesis, cag T4SS-dependent TLR9 activation was significantly impaired in the presence of the DNA gyrase inhibitor ciprofloxacin in a dose-dependent manner (FIG. 6A). To analyze the contribution of DNA segregation proteins to coupling transfer DNA to the cag T4SS apparatus, we employed targeted mutagenesis to delete individual genes known to be involved in DNA partitioning. Whereas isogeneic mutants deficient in genes encoding the DNA partitioning proteins ParA or ParB induced WT levels of cag T4SS-dependent TLR9 activation, isogenic mutants lacking genes encoding the DNA translocase ftsK (hp1090) or the recombinase xerH (hp0675) exhibited marked defects in TLR9 stimulation (FIG. 6B), suggesting that chromosomal dimer resolution is required for transfer DNA coupling to the cag T4SS apparatus. TLR9 stimulation defects exhibited by the ftsK mutant could be rescued by genetic complementation with full length FtsK, but not a truncated FtsK harboring only the translocase DNA binding domain (FtsK-γ, which is required for interaction with XerH and XerH-mediated DNA recombination (Debowski et al., 2012) (FIG. 6B). Loss of xerH or ftsK was not associated with defects in cag T4SS-dependent induction of IL-8 synthesis by gastric epithelial cells (FIG. 6C), indicating that chromosomal segregation defects specifically impair cag T4SS phenotypes associated with DNA translocation. Consistent with the observed defects in TLR9 activation, ftsK mutants stimulated significantly reduced levels of cGAS-STING signaling compared to WT and the corresponding FtsK complemented strain (FIG. 6D).

To investigate the role of chromosomal segregation in loading effector DNA into the cag T4SS apparatus, we developed a ‘transfer DNA’ immunopurification assay to capture chromosomal fragments contained within the cag T4SS translocation channel. We reasoned that effector DNA trafficked into the cag T4SS apparatus would physically interact with components of the outer membrane complex that comprise the secretion chamber (Chung et al., 2019; Frick-Cheng et al., 2016). To test this hypothesis, we treated H. pylori with formaldehyde to chemically cross-link DNA-protein complexes, lysed the cells by sonication, and purified the cag T4SS core complex via immunopurification targeting CagY, which forms the central cap-like structure within the inner ring of the outer membrane complex (Chung et al., 2019). PCR analysis targeting a 795 bp chromosomal fragment revealed the presence of DNA in reverse cross-linked CagY complexes purified from WT but not in mock preparations obtained from cagY isogenic mutants (FIG. 6E). DNA co-purification with CagY was not dependent on the presence of either the effector protein CagA or CagT, a component localized to the periphery of the outer membrane ring complex (Chung et al., 2019; Frick-Cheng et al., 2016); however, DNA loading into the cag T4SS apparatus was significantly impaired in the absence of FtsK (FIG. 6E). Together, these data demonstrate that effector DNA is loaded into the cag T4SS machinery prior to encountering host cells to establish a ‘ready-to-fire’ nanomachine and demonstrate that transfer DNA loading is mechanistically coupled to chromosomal replication and replichore decatenation. We thus propose a model in which FtsK-XerH complexes resolve imbalanced replichores arising from overlapping rounds of chromosomal replication, resulting in the rare excision of fragmented DNA that is subsequently shuttled the cag T4SS apparatus via unknown mechanisms (FIG. 6F).

### Discussion

STING-dependent immunosurveillance plays a critical role in maintaining gastric mucosal homeostasis and regulating inflammatory responses with carcinogenic potential (Ke et al., 2022). STING signaling has been implicated in the development and progression of stomach cancer, but whether STING activation promotes or restricts gastric carcinogenesis remains unresolved. For example, a previous study demonstrated that STING downregulation in primary gastric tumors was associated with increased malignancy and the progression of gastric cancer (Song et al., 2017), while a conflicting report determined that high STING expression in malignant tissues and tumor-associated macrophages was predictive of poor prognosis in stomach cancer patients (Miao et al., 2020). Thus, the molecular role of cGAS-STING signaling in chronic gastric inflammation and pre-malignant lesion development remains unresolved. Consistent with the observation that chronic H. pylori colonization elicits STING signaling in the murine gastric mucosa (Song et al., 2017), our study establishes a critical role of cag T4SS-mediated trans-kingdom DNA conjugation in stimulating STING-dependent outcomes that underscore infection-associated carcinogenesis.

In the context of bacterial infection, type I IFN responses have been associated with both protective and detrimental outcomes (Boxx and Cheng, 2016; Peignier and Parker, 2021). Previous work demonstrates that the H. pylori cag T4SS-dependent delivery of muropeptide fragments activates Nod1 sensing and IRF7-mediated type I IFN responses in epithelial cells that restrict bacterial proliferation via CXCL10-mediated immune cell recruitment (Viala et al., 2004; Watanabe et al., 2010). Our work demonstrates that during acute infection, CXCL10 is highly upregulated by cag T4SS activity in primary gastric epithelial cells (FIG. 3).

In addition to increased transcript levels associated with interferon stimulated genes, we observed augmented production of transcripts associated with SMHD1 [a suppressor of type I IFN and NF-_{κ}B signaling (Chen et al., 2018)], IFI35 [a negative regulator of RIG-I signaling (Das et al., 2014)], and ISG15 [an IFN-α/β-inducible ubiquitin-like modifier that is a key negative regulator of IFN-α/β immunity (Zhang et al., 2015)] in WT-infected primary gastric cells, suggesting that H. pylori has evolved mechanisms to counteract or suppress nucleic acid signaling pathways induced by cag T4SS activity. We speculate that similar to evasion strategies employed by numerous viruses, H. pylori counteracts type I IFN responses through the cag T4SS-dependent translocation of as yet unidentified, evolutionarily-conserved protein effectors that target and neutralize components of DNA-sensing or STING signaling pathways. Alternatively, pro-inflammatory STING signaling may balance immunosuppressive responses stimulated by cag T4SS-dependent TLR9 activation (Varga et al., 2016a) as a mechanism to sustain gastric homeostasis during acute colonization

Epithelial DNA damage, genomic alterations, and chromosomal instability are hallmarks of H. pylori-induced gastric cancer (Chaturvedi et al., 2014). While H. pylori cag T4SS activity has been shown to both induce the formation of double-stranded DNA breaks (DSBs) and micronuclei that potentially contribute to cGAS-STING signaling in gastric epithelial cells (Bauer et al., 2020; Hanada et al., 2014; Koeppel et al., 2015), our data suggest that translocated chromosomal DNA fragments serve as the predominant cytosolic cGAS trigger in the context of H. pylori infection (FIG. 1 and FIG. S1). In support of our data indicating that translocated chromosomal fragments are the key agonist that alerts cytosolic nucleic acid surveillance, we found that H. pylori effector DNA is packaged into exosomes released by gastric epithelial cells to enable paracrine-like signal amplification (FIG. 4). Consistent with a previous study demonstrating that intracellular Listeria monocytogenes infection stimulates the production of extracellular vesicles with IFN-inducing potential (Nandakumar et al., 2019), the delivery of pathogen-derived DNA via exosome release may represent a conserved mechanism by which epithelial cells potentiate nucleic acid surveillance-dependent danger signaling to uninfected tissues. We propose that H. pylori has evolved to exploit this innate defense mechanism to shape a tolerogenic immune response that favors persistent gastric colonization (Varga et al., 2016a).

Mechanistically, our data demonstrate that cag T4SS-dependent DNA delivery is coupled to chromosomal replication and replichore decatenation (FIG. 5 and FIG. 6). Similar to Neisseria gonorrhoeae ssDNA secretion mechanisms (Callaghan et al., 2017), chromosomal partitioning influences cag T4SS-mediated DNA translocation. Indeed, our studies reveal that effector DNA coupling to the cag T4SS apparatus requires both FtsK membrane tethering and DNA translocase activity (FIG. 6). In support of the critical role of replichore decatenation in cag T4SS-dependent DNA translocation, H. pylori lacking the DNA recombinase XerH, which requires direct interaction with FtsK to resolve chromosome dimers and catenated chromosomes (Bebel et al., 2016; Debowski et al., 2012), exhibited significantly reduced levels of TLR9 stimulation. Our studies suggest that while xerH mutants harbor increased DNA content per cell compared to the parental WT strain (Debowski et al., 2012), cag T4SS DNA translocation phenotypes are significantly impaired when chromosomal DNA topological isomers cannot be efficiently resolved (FIG. 6). Additionally, our findings establish that DNA effector molecules are pre-loaded into the cag T4SS apparatus to establish a ‘ready-to-fire’ nanomachine that can be rapidly deployed during acute infection, and demonstrate that FtsK is required for partitioning DNA substrates into the cag T4SS (FIG. 6). Although H. pylori harbors several orthologs to paradigmatic DNA conjugation systems, endonucleases, phage integrases, and canonical A. tumefaciens VirD2 relaxases, the mechanism by which chromosomally-derived effector DNA is precisely excised and shuttled through the cag T4SS while maintaining faithful genome inheritance remains unresolved. Thus, further studies are warranted to explore the intriguing possibility that the cag T4SS has co-opted orphaned components of remnant tfs3 and tfs4 conjugation systems to enable trans-kingdom DNA delivery (Fernandez-Gonzalez and Backert, 2014).

Our results provide the first direct evidence that H. pylori delivers immunostimulatory chromosomal DNA fragments into target epithelial cells and demonstrate that translocated effector DNA physically binds and activates cytosolic DNA-sensing reconnaissance systems. The observation that chromosomal fragments encompassing full-length eukaryotic genes can be excised and directed to the cag T4SS for delivery into target cells underscores the potential to exploit H. pylori trans-kingdom conjugation for targeted DNA cargo delivery. Notably, our work provides the essential experimental framework required to harness the cag T4SS as a mucosal-targeted DNA vaccine or therapeutic delivery device that can be pharmacologically controlled in vivo. Studies to determine the maximal DNA fragment length that can be efficiently transported to gastric cells via cag T4SS mechanisms are currently underway.

Our work highlights the importance of understanding host-pathogen conflicts that engage cellular PRR signaling axes to drive chronic inflammation and stimulate the development of infection-related malignancies. While we propose that translocated microbial DNA plays a critical and underappreciated role in the development of inflammation-associated carcinogenesis, further investigations are required to understand STING-dependent outcomes within the context of gastric cancer. Our findings identify a central role of cag T4SS activity in eliciting cGAS-STING immunosurveillance in the gastric epithelium; however, additional studies are necessary to understand how STING activation shapes the gastric immune landscape to enable persistent H. pylori colonization and to elucidate whether STING signaling influences the development of pre-malignant lesions. Finally, our work more broadly points towards therapeutic STING modulation as a promising intervention strategy to reduce the incidence and severity of infection-associated malignancies that arise as a consequence of chronic inflammation.

### Helicobacter pylori Type IV Secretion System Cross Talk Enables Trans-Kingdom DNA Conjugation

Recognition of foreign nucleic acids is an evolutionarily conserved mechanism by which the host detects microbial threats. Whereas some intracellular bacterial pathogens trigger DNA surveillance pathways following phagosomal membrane perturbation, mechanisms by which extracellular bacteria activate cytosolic nucleic acid reconnaissance systems remain unresolved. Here, we demonstrate that Helicobacter pylori exploits cag type IV secretion system (cag T4SS) activity to provoke STING signaling in gastric epithelial cells. We provide direct evidence that immunostimulatory chromosomal fragments delivered to the host cell cytoplasm bind and activate the DNA sensor cGMP-AMP synthase and determined that double-stranded DNA is a specific cag T4SS nucleic acid substrate. We identified several ‘uncoupling mutations’ that selectively disarm DNA translocation and established that cag T4SS-dependent DNA delivery is mechanistically coupled to chromosomal replication and replichore decatenation. We discovered that interplay between distinct T4SS machineries harbored by H. pylori enables trans-kingdom conjugation through a mechanism whereby an endonuclease associated with the tfs3 conjugative T4SS excises effector DNA and interfaces with the cag T4SS to facilitate cargo delivery. Finally, we show that DNA is coupled to the cag T4SS apparatus prior to host cell contact to establish a ‘ready-to-fire’ molecular device and demonstrate that DNA loading is dependent upon topological isomer resolution and orthologous relaxase tethering to the cag T4SS translocation channel. Collectively, our study highlights how the cag T4SS nanomachine evolved to co-opt orphaned components of divergent DNA transfer systems and chromosomal partitioning machineries to diversify the effector repertoire and expand apparatus versatility.

While intracellular bacterial pathogens elicit cGAS-STING signaling following phagosomal membrane destabilization or rupture achieved in a type III, IV, VI, or VII secretion system-dependent manner (Ku et al., 2020; Marinho et al., 2017; Nandakumar et al., 2019; Storek et al., 2015; Watson et al., 2015; Zhang et al., 2014), the mechanisms by which extracellular bacteria stimulate DNA reconnaissance systems remain unclear. H. pylori harbors up to four chromosomally-encoded T4SS machineries with the putative capacity to transport DNA into and out of the bacterial cell—(i) the cag T4SS, (ii) the comB DNA uptake system required for natural competence, and the (iii) tfs3 and (iv) tfs4 interbacterial DNA conjugation systems (Fischer et al., 2020). Canonical trans-kingdom conjugation involving transfer of plasmid-derived ssDNA into plant cells by the Agrobacterium tumefaciens vir T4SS requires the cognate VirD2 relaxase and VirD4 coupling protein that recruits the relaxosome to the T4SS apparatus (Christie et al., 2005; Costa et al., 2021). While the cag T4SS harbors an orthologous VirD4-like coupling protein that is essential for CagA delivery but dispensable for cag T4SS-mediated TLR9 stimulation (Lin et al., 2020), VirD2-like endonucleases are not encoded within the cag T4SS pathogenicity island (Fischer et al., 2020). The observation that tfs3 and tfs4 plasticity zones encode putative origin of transfer (oriT) sequences, conjugational relaxases related to the prototypical A. tumefaciens VirD2 endonuclease, and VirD4-like coupling proteins (Costa et al., 2021; Fischer et al., 2020; Kersulyte et al., 2009) raised the hypothesis that T4SS cross talk enables cag T4SS-dependent trans-kingdom conjugation.

Here, we demonstrate that H. pylori-induced cGAS-STING signaling is a direct consequence of cag T4SS-dependent DNA translocation. We provide evidence that in contrast to A. tumefaciens, the H. pylori cag T4SS delivers double-stranded DNA (dsDNA) fragments derived from the bacterial chromosome into host cells via trans-kingdom conjugation. We show that translocated DNA cargo is sorted into exosomes released by primary gastric epithelial cells to stimulate DNA-sensing pathways in uninfected bystanders as a mechanism to potentiate inflammatory responses via paracrine-like signaling. We demonstrate that cag T4SS-mediated DNA translocation is mechanistically coupled to chromosomal replication and topological isomer disentanglement, and show that designer DNA constructs optimized for eukaryotic expression are delivered to the gastric epithelial cell via cag T4SS mechanisms. We provide evidence that cag T4SS-mediated DNA translocation occurs through a two-step process in which dsDNA is at least partially exposed to the extracellular milieu prior to entering the host cell cytoplasm, suggesting that in contrast to prototypical conjugative transfer models, cag T4SS DNA is not secreted through a pilus conduit into the target cell. Finally, we identify the tfs3-associated VirD2-like endonuclease Hp0996 as a critical DNA translocation factor that excises target chromosomal sequences and couples transfer DNA to the cag T4SS secretion channel for transport via trans-kingdom conjugation. To our knowledge, this is the first report of an endogenous T4SS chimera in which component interchangeability affords expanded cag T4SS apparatus versatility and effector molecule diversity. Collectively, our results highlight how H. pylori exploits cag T4SS mosaicism to tip the delicate STING signaling balance towards inflammatory responses that may stimulate carcinogenesis and enable chronic colonization of the gastric niche.

### Results

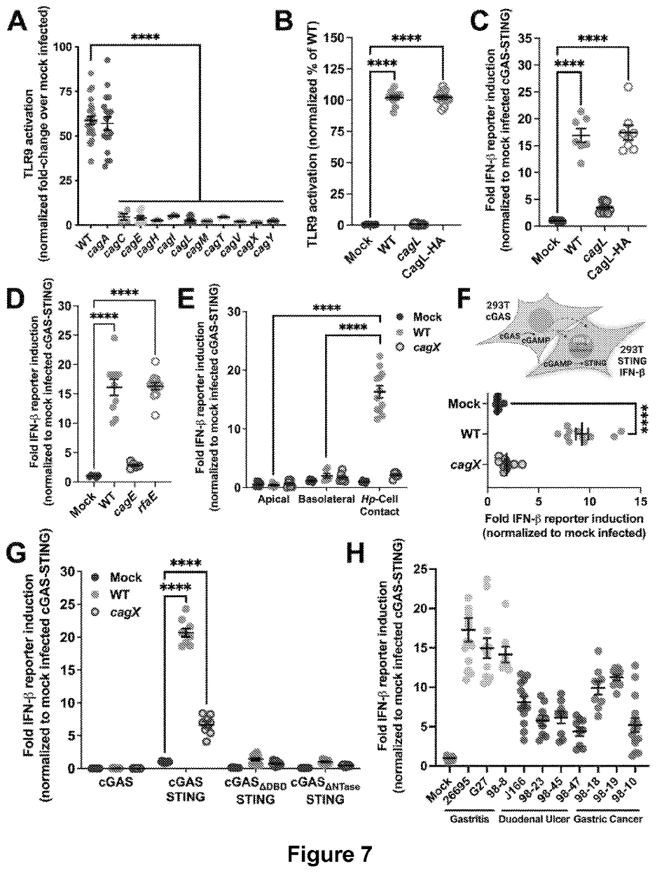

H. pylori provokes multiple DNA surveillance systems in a cag T4SS-dependent manner. Previous studies demonstrate the capacity of H. pylori to stimulate DNA-sensing pattern recognition receptors, including TLR9 (Rad et al., 2009; Varga et al., 2016b), raising the hypothesis that microbial nucleic acids are actively translocated into host cells. In agreement with previous reports (Rad et al., 2009; Varga et al., 2016b), H. pylori challenge of HEK293 reporter cell lines stably transfected with TLR9 demonstrated that cag T4SS activity is required for TLR9 stimulation (FIG. 7A,B). Consistent with the observation that CagA is not translocated into HEK293 cells (Kumar Pachathundikandi et al., 2011; Varga et al., 2016b), disruption of cagA did not diminish levels of H. pylori-induced TLR9 activation (FIG. 7A). To confirm that a functional cag T4SS is required to activate TLR9, we co-cultured TLR9 reporter cells with a cagL isogenic mutant or the corresponding genetically complemented strain. Whereas inactivation of cagl abrogated TLR9 activation, complementation in a heterologous chromosomal locus rescued TLR9 stimulation to levels indistinguishable from the parental WT strain (FIG. 7B). In concert with previous investigations, these data demonstrate that cag T4SS activity, but not CagA delivery, is required for robust TLR9 signaling.

In addition to endosomal DNA pattern recognition receptors such as TLR9, gastric epithelial cells harbor cytosolic DNA surveillance proteins including the nucleotidyltransferase cyclic GMP-AMP (cGAMP) synthase (Cai et al., 2014; Diner et al., 2013; Sun et al., 2013; Wu et al., 2013). To test whether H. pylori DNA is trafficked into the host cell cytoplasm to activate cytosolic DNA surveillance sensors, we challenged 293T cells transfected with constructs to enable cGAS and STING expression and quantified levels of IFN-β promoter-driven luciferase produced in response to H. pylori. Compared to mock infected cells, WT H. pylori stimulated high levels of cGAS-STING signaling at 18 h post-infection. Similar to TLR9 activation assays, the cagL mutant was unable to stimulate robust cGAS-STING signaling, a phenotype that was restored by genetic complementation in a secondary chromosomal locus (FIG. 7C). In contrast to HEK293-hTLR9 cells, moderate levels of translocated CagA were detected in 293T cells co-cultured with WT H. pylori but not in corresponding cagE-challenged monolayers. Although H. pylori has the capacity to deliver CagA into 293T cells, disruption of cagA did not significantly alter levels of cag T4SS-dependent IFN-β promoter activity in cGAS-STING reporter cells. To exclude the possibility that cGAS-STING activation results from bacterial endocytosis or increased bacterial interaction with host cell surfaces, we performed gentamicin protection assays. In contrast to marked differences in cGAS-STING activation elicited by WT and cagX, equivalent levels of adherent and intracellular bacteria were recovered from 293T co-cultures, confirming that cGAS-STING signaling is not an artifact of non-specific bacterial internalization or spontaneous bacterial lysis.

Previous studies demonstrate that in addition to detecting invading microbial threats, cGAS senses and responds to DNA damage and genomic instability (Cai et al., 2014; Ke et al., 2022). H. pylori cag T4SS activity is directly linked to nuclear double-stranded DNA (dsDNA) breaks introduced in response to ALPK1/TIFA signaling stimulated by D-glycero-beta-D-manno-heptose 1,7-bisphosphate (HBP) or ADP-beta-D-manno-heptose (β-ADP-heptose) translocation (Bauer et al., 2020; Gall et al., 2017; Zimmermann et al., 2017). We therefore addressed the possibility that cag T4SS-dependent nuclear DNA damage stimulates cGAS-STING signaling elicited by WT H. pylori. Disruption of rfaE, the enzyme required for HBP and β-ADP-heptose production (Bauer et al., 2020; Gall et al., 2017; Stein et al., 2017; Zimmermann et al., 2017), did not impact the level of cGAS-STING signaling achieved by H. pylori (FIG. 7D), indicating that ALPK1/TIFA signaling-induced DNA damage does not significantly contribute to H. pylori-driven cGAS activation in vitro. In addition to ALPK1/TIFA-mediated DNA modifications (Bauer et al., 2020; Gall et al., 2017; Stein et al., 2017; Zimmermann et al., 2017), H. pylori has the capacity to induce production of DNA-damaging reactive oxygen species (ROS) by triggering NF-KB activation and additional mechanisms involving inducible nitric oxide synthase (iNOS) and associated inflammatory enzymes (Bauer et al., 2020; Kidane, 2018). In support of the observation that cag T4SS-induced dsDNA breaks are not a significant cGAS activating factor in the context of H. pylori infection, cGAS-STING activation was achieved by H. pylori co-cultured in the presence of the antioxidant N-acetyl-cysteine at concentrations that abrogate ROS production (Bauer et al., 2020). Following infection, damaged mitochondria release DNA (mtDNA) and other constituents into the cytosol to act as potent danger-associated molecular patterns (DAMPs) that engage TLR9 (Garcia-Martinez et al., 2016; Oka et al., 2012; Zhang et al., 2010) and cGAS-STING signaling axes to initiate type I IFN production (Rongvaux et al., 2014; West et al., 2015; White et al., 2014). To test the hypothesis that H. pylori cag T4SS activity modulates mitochondrial integrity resulting in the release of mtDNA and activation of cytosolic DNA-sensing PRRs, we assayed cGAS-STING activation in the presence of BAX/BAK macropore inhibitory peptides that prevent permeabilization of the mitochondrial outer membrane and herniation of mtDNA into the cytosol (McArthur et al., 2018; White et al., 2014). H. pylori induced similar levels of cGAS-STING activation in the presence of BAX inhibitory peptide or non-inhibitory peptide control co-cultures suggesting that ruptured mitochondria are not the primary source of cGAS-activating DNA. Likewise, treatment of co-cultures with a mitochondria-targeted antioxidant did not significantly alter levels of cGAS-STING signaling, demonstrating that H. pylori-induced mtDNA damage is not a predominant DAMP within the context of cag T4SS-dependent cGAS activation. To exclude the possibility that cGAS-STING activation is dependent upon the import of released H. pylori DNA via host cell mechanisms, we quantified IFN-β promoter activity produced by cGAS-STING reporter cells that were physically separated from H. pylori. In support of the hypothesis that cGAS-STING activation requires direct H. pylori-host cell interaction, cGAS stimulation was achieved by H. pylori that were in direct contact with reporter cells, but not in samples in which H. pylori and reporter cells were physically separated by a 0.4 μM-pore polycarbonate insert (FIG. 7E). Collectively, these studies demonstrate that cGAS stimulation is a consequence of cag T4SS-dependent DNA delivery into the host cell cytosol.

To define the role of cGAS in H. pylori-driven IFN-β signaling, we next determined whether cag T4SS activity could transactivate STING through direct cGAMP transfer via gap junction-mediated diffusion. To assess cGAS signaling in trans, 293T cells transfected with cGAS constructs were co-cultured with 293T cells harboring STING and IFN-β reporter constructs, and co-cultures were challenged by H. pylori for 18 h. High levels of STING signaling were observed in cells challenged by WT H. pylori, but not a cagX mutant, suggesting that cag T4SS-dependent cGAS stimulation generates sufficient cGAMP for migration into bystander cells (FIG. 7F). To examine the requirement of cGAS protein domains in H. pylori-induced STING signaling, we next monitored IFN-β activation in 293T cells transfected with constructs to express either cGAS alone or cGAS variants in combination with STING. In comparison to cells transfected with only cGASWT, WT H. pylori stimulated high levels of IFN-β transcription when cGASWT and STING were co-expressed (FIG. 7G). In contrast, cag T4SS-dependent IFN-β transcription was markedly reduced in 293T cells transfected with STING and cGAS variants lacking the DNA-binding domain (cGAS_{ΔDBD}) or harboring point mutations within nucleotidyltransferase catalytic residues that abolish cGAMP production (cGAS_{ΔNTase}) (FIG. 7G). To evaluate the kinetics of cGAS-STING activation, we used co-culture systems paired with gentamicin treatment at defined time points. In these assays, we observed robust reporter transcription occurring within 4 to 6 hours post-infection in WT-challenged co-cultures, with IFN-b reporter transcripts accumulating steadily over time. Collectively, these results demonstrate that cGAS senses and responds to cag T4SS activity.

A previous report suggests that decreased STING signaling is associated with adverse outcomes in gastric cancer patients (Song et al., 2017). We therefore monitored cGAS-STING signaling induced by several cag T4SS-positive H. pylori clinical isolates obtained from patients with gastric diseases of varying severity. In contrast to strains isolated from patients exhibiting gastritis, H. pylori isolated from individuals with duodenal ulcers or gastric cancer elicited lower levels of cGAS-STING signaling (FIG. 7H), suggesting that H. pylori may modulate the capacity to induce cag T4SS-dependent STING activation during chronic stomach colonization.

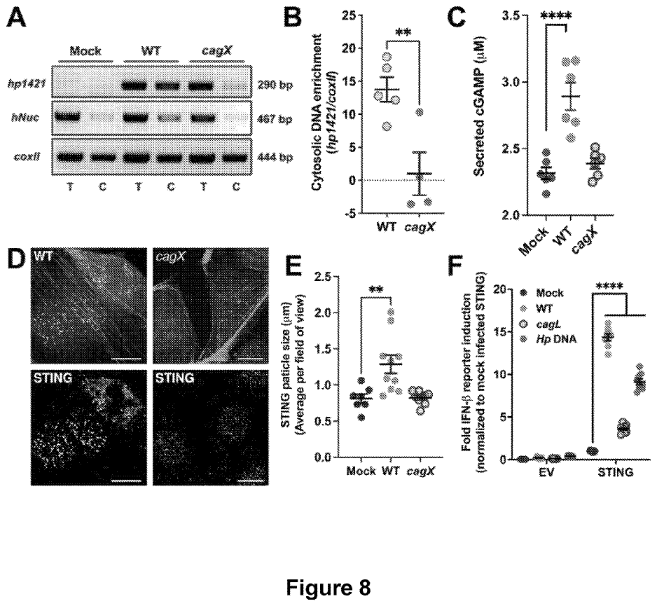

Microbial DNA is delivered to the gastric epithelial cell cytoplasm via cag T4SS activity. We next assessed the consequence of H. pylori trans-kingdom DNA conjugation within the context of biologically-relevant interactions with gastric epithelial cells. To monitor DNA injection into gastric epithelial cells, AGS cells were challenged by either WT or the cagX isogenic mutant at a MOI of 50. After 6 h, gastric epithelial cell co-cultures were treated with DNasel to remove extracellular DNA and eukaryotic cells were fractionated using digitonin to selectively permeabilize the plasma membrane, leaving the nuclear envelope and bacterial cells intact. PCR analysis of fractionated infected AGS cells revealed the presence of H. pylori DNA in cytoplasmic fractions (FIG. 8A) with significantly more bacterial DNA present in cytosolic fractions obtained from WT-challenged cells. When comparing the ratio of cytoplasmic bacterial DNA to cytoplasmic-localized mitochondrial DNA by qPCR, significantly more bacterial DNA was present in the cytosol of gastric epithelial cells infected by the WT strain compared to corresponding cells infected by the cagX mutant (FIG. 8B). Levels of mitochondrial DNA did not significantly differ in cytosolic extracts obtained from infected and uninfected cells (FIG. 8A). To exclude the possibility that cytosolic localization of H. pylori DNA resulted from non-specific bacterial lysis or endocytosis, we performed studies to analyze the level of adherent and internalized H. pylori in infected gastric epithelial cells. Using gentamicin-protection assays, we determined that similar levels of adherent and intracellular WT and cagX bacteria were recovered from AGS co-cultures (data not shown).

Multiple cancer cell lines, including cell lines derived from gastric adenocarcinoma, continuously export low levels of extracellular cGAMP that serves as a potent immunotransmitter (Carozza et al., 2020). We thus measured levels of extracellular cGAMP secreted by AGS cells in response to H. pylori challenge. Consistent with the hypothesis that cag T4SS-mediated DNA delivery stimulates cytosolic cGAS leading to the subsequent production of cGAMP, the level of extracellular cGAMP was significantly higher in supernatants obtained from WT-challenged co-cultures compared to mock infected or corresponding cagX-challenged monolayers (FIG. 8C). Collectively, these results suggest that the presence of bacterial DNA in the host cell cytoplasm is a consequence of cag T4SS activity and leads to the production of cGAS-catalyzed cGAMP.

Although gastric adenocarcinoma cell lines (including AGS and MKN45) produce detectable levels of cGAS and other nucleic acid-sensing PRRs, STING expression is absent (Qiao et al., 2020). To determine whether translocated H. pylori DNA elicits cGAS-STING responses in normal gastric epithelia, we challenged primary adult gastric epithelial cells with H. pylori and monitored the formation of peri-nuclear STING polymers that aggregate in response to cGAMP binding. Compared to mock infected or cagX-challenged cells, WT H. pylori induced the formation of large STING aggregates that could be visualized by confocal microscopy (FIG. 8D). Consistent with activation of cGAS-STING signaling, quantification of the average STING polymer size in H. pylori-gastric cell co-cultures revealed significantly larger STING aggregates in WT-challenged cells (FIG. 8E), suggesting cag T4SS-dependent stimulation of cGAS surveillance. In addition to cGAS, epithelial cells harbor other nucleic acid reconnaissance systems that signal through STING. To determine whether H. pylori stimulates additional STING-dependent signaling pathways, we analyzed IFN-β activity in 293T-STING cells. Compared to control 293T cells harboring empty vectors (EVs), H. pylori stimulated STING signaling in a cag T4SS-dependent manner (FIG. 8F). STING signaling was also elicited by transfection of purified, fragmented H. pylori chromosomal DNA into IFN-β reporter cells (FIG. 8F), suggesting that endogenous 293T cytosolic DNA-sensing pathways respond to H. pylori DNA. Together, these studies demonstrate that H. pylori delivers chromosomal DNA fragments to gastric epithelial cells via cag T4SS mechanisms and reveal that endogenous STING-dependent nucleic acid surveillance systems are activated by translocated H. pylori DNA.

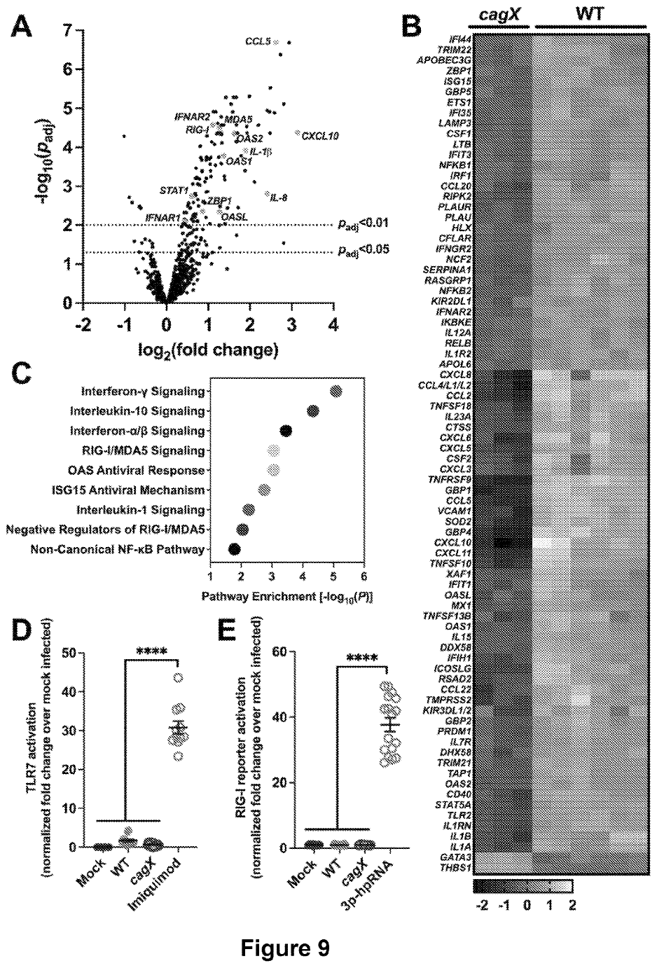

DNA is a specific cag T4SS nucleic acid substrate. We reasoned that in addition to DNA substrates, the cag T4SS may translocate RNA or DNA: RNA hybrids into gastric cells to stimulate STING-dependent signaling. Thus, we sought to characterize the innate inflammatory signature elicited by H. pylori. Primary adult gastric epithelial cells were mock infected or challenged by either WT or the corresponding cagX isogenic mutant prior to isolation of total RNA. To characterize epithelial innate inflammatory responses, gene expression patterns were analyzed at 6 h post-infection using the NanoString Host Response Panel. Compared to mock-infected and cagX-challenged primary gastric epithelial cell co-cultures, cag T4SS activity induced a significant increase in transcripts associated with anti-microbial defense pathways and interferon signaling (FIG. 9A). Hierarchical clustering performed on genes that were differentially expressed in response to H. pylori challenge revealed distinct clustering of mock infected and cagX-infected co-cultures in comparison to WT challenge. Comparison of transcripts differentially produced in response to WT versus cagX H. pylori identified 134 genes that were upregulated via cag T4SS activity, including genes encoding pro-inflammatory chemokines and cytokines (e.g., CXCL10, CXCL8/IL-8, TNF, IL1B, CCL5), IFN-α/β signaling (e.g., ISG15, IRF1, IFI35, STAT1, IFNAR2, SAMHD1), and IFN-γ immunoregulatory programs (e.g., IFNGR2, TRIM5, GBP4, VCAM1) (FIG. 9A,B). Consistent with the observed gene expression patterns, pathway analysis of genes induced by cag T4SS activity demonstrated a significant enrichment in transcripts associated with interferon-regulated nucleic acid reconnaissance programs (FIG. 9C). In addition to cag T4SS-dependent regulation of the DNA sensor ZBP1, we observed significantly increased transcript levels of several cytosolic RNA-sensing surveillance systems including DDX58/RIG-I, RIG-I-like Receptor IFIH1/MDA5 (Chow et al., 2015; Dixit and Kagan, 2013), and 2′-5′-oligoadenylate synthetases OAS1 and OAS2 (Schwartz et al., 2020) in WT-infected primary gastric epithelial cells (FIG. 9A,B).

A previous study reported the capacity of gastric mucosa-associated dendritic cells to sense and respond to purified H. pylori RNA through RIG-I and TLR 7/8, leading to the production of type I IFN (Rad et al., 2009; Salama et al., 2013). However, whether the cag T4SS actively translocates RNA to stimulate TLR7/8 or RIG-I signaling axes has not been elucidated. We thus sought to determine whether DNA is a specific cag T4SS nucleic acid effector. Challenge of HEK293 reporter cell lines expressing either TLR7 (FIG. 9D) or RIG-1 (FIG. 9E) revealed that in contrast to robust activation of DNA-sensing PRRs, H. pylori was unable to activate either endosomal ssRNA (TLR7) or cytoplasmic ssRNA/dsRNA (RIG-I) sensors. Collectively, these data suggest that chromosomally-derived DNA is a specific cag T4SS substrate, and provide further evidence that cag T4SS-mediated DNA translocation stimulates STING-dependent signaling.

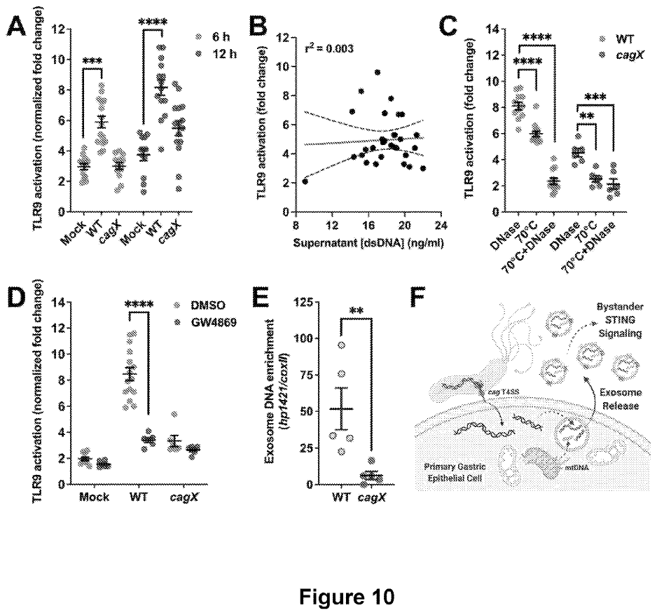

H. pylori cag T4SS activity stimulates DNA immunosurveillance in bystander cells. Prior work demonstrates that foreign intracellular bacterial DNA can be delivered to adjacent cells via extracellular vesicles as a mechanism to amplify IFN signaling (Nandakumar et al., 2019). We therefore investigated whether translocated H. pylori DNA could be sorted into extracellular vesicles to enable paracrine-like DNA signaling by primary gastric epithelial cells. To test the hypothesis that infected gastric epithelial cells release extracellular vesicles containing H. pylori DNA, cell-free supernatants collected from infected primary gastric epithelial donor cells were used to challenge recipient TLR9 reporter cells. Compared to supernatants obtained from either mock infected or cagX-challenged co-cultures, supernatants harvested from WT-challenged gastric cells robustly activated TLR9 signaling (FIG. 10A). The capacity of infected cell supernatants to activate TLR9 was time dependent, as levels of TLR9 activation increased when reporter cells were treated with supernatants obtained from gastric epithelial cells co-cultured with H. pylori for 12 h compared to 6 h (FIG. 10A). To exclude the possibility that TLR9 activation resulted from contaminate DNA arising from cell lysis, we quantified the level of cell-free DNA in donor supernatants. TLR9 activation elicited by gastric cell supernatants was not correlated to the level of donor supernatant cell-free DNA (FIG. 10B), suggesting that the TLR9-stimulating agonist was enclosed within a host cell-derived delivery mechanism.

To confirm the role of translocated DNA in transferrable nucleic acid immunosurveillance in bystander cells, donor gastric cell supernatants were treated with DNase alone or in combination with heat prior to co-culture with TLR9 reporter cells. Whereas DNase treatment had a negligible effect on TLR9 stimulating capacity, heat treatment modesty reduced TLR9 responses, which were further reduced when donor supernatants were treated with both heat and DNase prior to reporter cell challenge (FIG. 10C). These observations led to the hypothesis that translocated microbial DNA packaged within gastric cell-derived extracellular vesicles, such as exosomes, stimulates nucleic acid reconnaissance systems in bystander cells. To test whether exosome biogenesis is required for the delivery of translocated DNA to uninfected cells, we challenged recipient TLR9 reporter cells with donor supernatants obtained from infected primary gastric epithelial cells cultured in the presence or absence of a neutral sphingomyelinase (nSmase2) inhibitor that prevents exosome release. Compared to untreated and mock infected cells, nSmase2 inhibition led to a significant reduction in TLR9 activation levels elicited by WT-challenged donor cell supernatants (FIG. 10D), supporting a role of exosomes in stimulating DNA-sensing pathways in bystander cells. To further investigate whether translocated H. pylori DNA was packaged within exosomes released from infected gastric epithelial cells, we isolated CD9, CD63, and CD81-positive exosomes from cell culture supernatants via immunopurification. Analysis by qPCR revealed that exosomes purified from WT-challenged gastric cell supernatants were significantly enriched in H. pylori DNA compared to corresponding exosomes obtained from cagX-challenged co-cultures (FIG. 10E), suggesting that foreign bacterial DNA is sorted into exosomes for paracrine-like DAMP signal amplification (FIG. 10F).

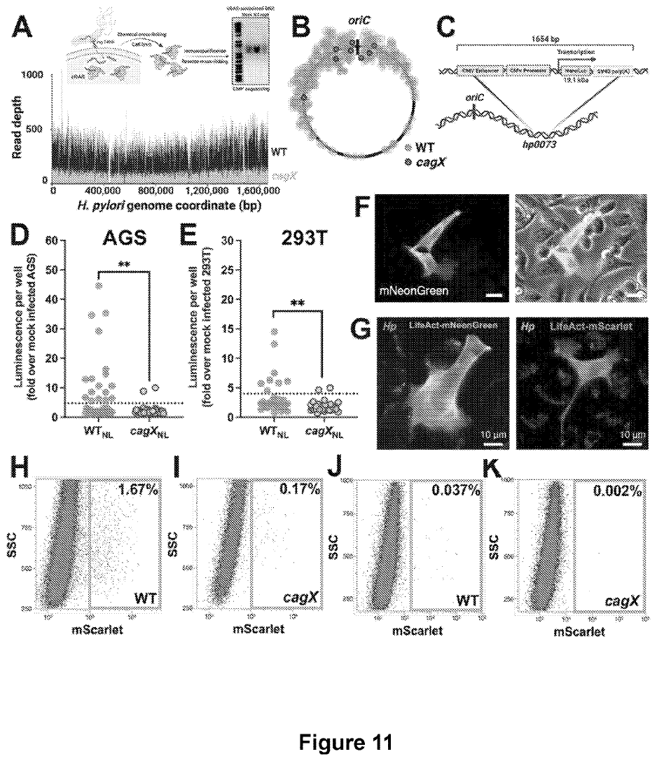

Chromosomal fragments are delivered to target cells via cag T4SS-dependent mechanisms. To identify whether a specific DNA sequence is excised and transferred to host cells by cag T4SS activity, we purified cGAS-DNA complexes using modified ChIP-seq workflows to capture bacterial DNA that physically binds and activates cGAS. 293T-cGAS cells were challenged by H. pylori for 6 h, followed by chemical cross-linking and immunopurification of cGAS. Deep sequencing analysis confirmed that co-purifying H. pylori DNA isolated from WT and cagX-infected cells mapped across the entire H. pylori chromosome, with significantly more bacterial DNA reads associated with cGAS purifications obtained from WT-challenged monolayers (FIG. 11A). Normalized sequencing reads and differential peak calling approaches identified more than three hundred H. pylori chromosomal regions specifically associated with cGAS purified from WT-infected cells compared to eight bacterial DNA peaks associated with corresponding preparations obtained from cagX co-cultures (P=0.01). When enriched peak centers were mapped to the coordinate position across the H. pylori chromosome, differentially enriched peaks heavily clustered around the oriC region, with few peaks mapping to the chromosomal region diametrically opposed to oriC (FIG. 11B), suggesting that DNA translocation is linked to bi-directional DNA replication.

To test the hypothesis that chromosomal regions near oriC are translocated to gastric epithelial cells at a high frequency, we cloned a CMV promoter-driven monomeric nanoluciferase construct into the ureA locus (hp0073) adjacent to oriC (FIG. 11C). Gastric epithelial cells and 293T cells were challenged by WT or cagX H. pylori strains harboring the eukaryotic-optimized nanoluciferase expression construct (WTNL and cagXNL, respectively), and luciferase activity in infected cell lysates was monitored by bioluminescence. Compared to mock infected and cagXNL-infected cells, AGS and 293T cells challenged by WTNL produced high levels of bioluminescence (FIGS. 11D and 11E), indicating that nanoluciferase production by epithelial cells is linked to cag T4SS-dependent DNA translocation.

To confirm that targeted DNA fragments proximal to oriC can be transferred to host cells via cag T4SS mechanisms, we replaced the nanoluciferase gene with a eukaryotic-optimized construct designed to express monomeric mScarlet tethered to the LifeAct N-terminal peptide to enable live cell visualization of F-actin. Microscopy analysis of AGS (FIGS. 11F) and 293T (FIG. 11G) monolayers challenged by WTmScarlet revealed numerous mScarlet-positive cells compared to corresponding cagXmScarlet-infected co-cultures. Flow cytometry analysis confirmed our observation that significantly more mScarlet-positive epithelial cells were generated when monolayers were challenged by WTmScarlet compared to T4SS-deficient controls (FIG. 11H-K). Together, these data provide direct evidence that targeted chromosomal fragments greater than ~2 kb in length are excised and delivered to host cells via cag T4SS-dependent mechanisms.

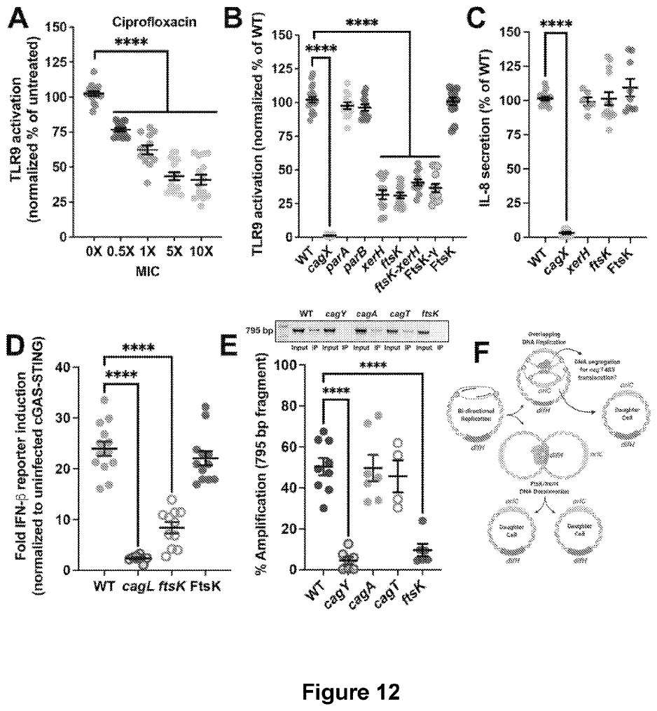

H. pylori trans-kingdom conjugation is mechanistically coupled to chromosomal decatenation. We next sought to understand the mechanism by which effector DNA is coupled to the cag T4SS for transfer to target host cells. The observation that fragments of translocated bacterial DNA map predominantly to the oriC region led to the hypothesis that H. pylori trans-kingdom conjugation is linked to chromosomal replication. In support of this hypothesis, cag T4SS-dependent TLR9 activation was significantly impaired in the presence of the DNA gyrase inhibitor ciprofloxacin in a dose-dependent manner (FIG. 12A). To analyze the contribution of DNA segregation proteins to coupling transfer DNA to the cag T4SS apparatus, we employed targeted mutagenesis to delete individual genes known to be involved in DNA partitioning. Whereas isogeneic mutants deficient in genes encoding the DNA partitioning proteins ParA or ParB induced WT levels of cag T4SS-depenedent TLR9 activation, isogenic mutants lacking genes encoding the DNA translocase ftsK (hp1090) or the recombinase xerH (hp0675) exhibited marked defects in TLR9 stimulation (FIG. 12B), suggesting that chromosomal dimer resolution is required for transfer DNA coupling to the cag T4SS apparatus. TLR9 stimulation defects exhibited by the ftsK mutant could be rescued by genetic complementation with full length FtsK, but not a truncated FtsK harboring only the translocase DNA binding domain (FtsK-γ), which is required for interaction with XerH and XerH-mediated DNA recombination (Debowski et al., 2012) (FIG. 12B). Loss of xerH or ftsK was not associated with defects in cag T4SS-dependent induction of IL-8 synthesis by gastric epithelial cells (FIG. 12C), indicating that chromosomal segregation defects specifically impair cag T4SS phenotypes associated with DNA translocation. Consistent with the observed defects in TLR9 activation, ftsK mutants stimulated significantly reduced levels of cGAS-STING signaling compared to WT and the corresponding FtsK complemented strain (data not shown).

To investigate the role of chromosomal segregation in loading effector DNA into the cag T4SS apparatus, we developed a ‘transfer DNA’ immunopurification assay to capture chromosomal fragments contained within the cag T4SS translocation channel. We reasoned that effector DNA trafficked into the cag T4SS apparatus would physically interact with components of the outer membrane complex that comprise the secretion chamber (Chung et al., 2019; Frick-Cheng et al., 2016). To test this hypothesis, we treated H. pylori with formaldehyde to chemically cross-link DNA-protein complexes, lysed the cells by sonication, and purified the cag T4SS core complex via immunopurification targeting CagY, which forms the central cap-like and periplasmic ring structures within the inner ring of the outer membrane complex (Chung et al., 2019). PCR analysis targeting a 795 bp chromosomal fragment revealed the presence of DNA in cross-linked CagY complexes purified from WT but not in mock preparations obtained from cagY isogenic mutants (FIG. 12E). DNA co-purification with CagY was not dependent on the presence of either the effector protein CagA or CagT, a component localized to the periphery of the outer membrane ring complex (Chung et al., 2019; Frick-Cheng et al., 2016); however, DNA loading into the cag T4SS apparatus was significantly impaired in the absence of FtsK (FIG. 12E). Together, these data demonstrate that effector DNA is loaded into the cag T4SS machinery prior to encountering host cells to establish a ‘ready-to-fire’ nanomachine and demonstrate that transfer DNA loading is mechanistically coupled to chromosomal replication and replichore decatenation. We thus propose a model in which FtsK-XerH complexes resolve imbalanced replichores arising from overlapping rounds of chromosomal replication to liberate excess DNA that is subsequently processed and shuttled through the cag T4SS apparatus (FIG. 12F).

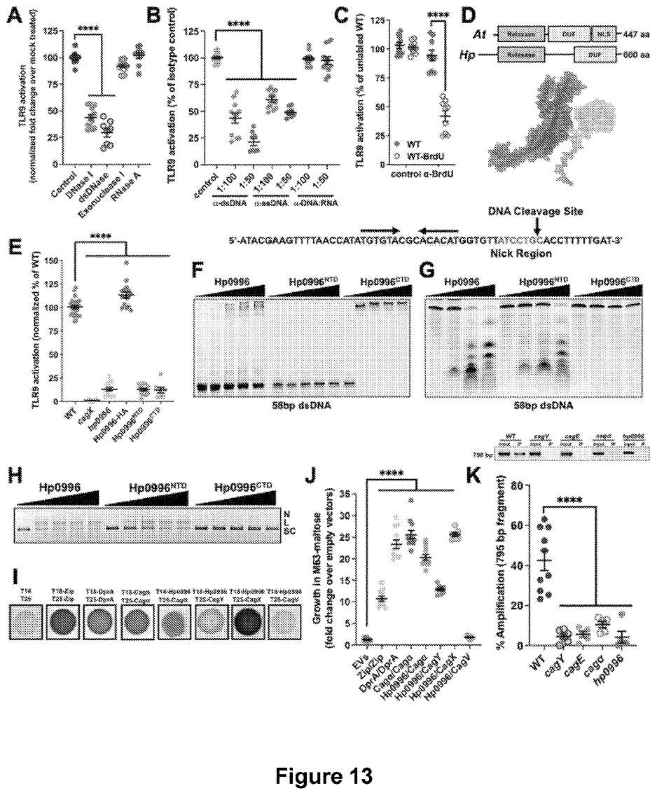

Chromosomal DNA is delivered to host cells via a novel secretion mechanism. We next performed studies to determine if translocated H. pylori DNA is protected from the extracellular environment (e.g., enclosed within a T4SS conduit) by assessing whether exogenous nuclease treatment would compromise DNA transport and thereby impair TLR9 activation. In agreement with a previous study (Varga et al., 2016b), H. pylori-driven TLR9 activation decreased by approximately 50% when DNase I was added to co-cultures concomitantly with H. pylori compared to levels achieved in the absence of exogenous nuclease (FIG. 13A). DNase I non-specifically cleaves single- and double-stranded DNA, as well as RNA: DNA hybrids. Therefore, we tested whether nucleases that specifically degrade either double-stranded DNA (dsDNase) or single-stranded DNA (exonuclease I) would impact cag T4SS-dependent DNA translocation. TLR9 activation was significantly impaired by the addition of exogenous dsDNase but was unaffected by the presence of exonuclease I or RNase A, supporting the hypothesis that translocated H. pylori DNA is double-stranded. In parallel, we analyzed the ability of monoclonal anti-DNA antibodies to functionally block TLR9 activation. Compared to isotype control treated co-cultures, H. pylori-driven TLR9 activation was significantly impaired in the presence of either anti-dsDNA or, to a lesser degree, anti-ssDNA monoclonal antibodies (FIG. 13B), a result likely due to inherent antibody cross-reactivity. In complementary studies, we further analyzed whether bacterial DNA is localized to an extracellular site prior to translocation into host cells by labeling the H. pylori chromosome via bromodeoxyuridine (BrdU) incorporation. Compared to unlabeled WT bacteria, anti-BrdU antibodies significantly impaired TLR9 activation by H. pylori that had incorporated BrdU nucleoside analogs into the chromosome (FIG. 13C). Collectively, these results suggest that H. pylori DNA cargo is at least partially double-stranded and localized to an extracellular site prior to entering host cells. Our data coalesce with previous results demonstrating that CagA translocation is partially blocked in the presence of anti-CagA monoclonal antibodies (Murata-Kamiya et al., 2010), supporting a model whereby cag T4SS cargo is transported to the bacterial cell surface prior to delivery to the gastric cell cytoplasm.

Trans-kingdom DNA delivery requires the VirD2-like endonuclease Hp0996. We hypothesized that in addition to FtsK/XerH and the cag T4SS, orphaned T4SS components may be required for trans-kingdom DNA delivery. To investigate the mechanism by which transfer DNA is excised and coupled to the cag T4SS apparatus, we next performed studies to determine whether tfs3- and tfs4-associated putative endonucleases, tyrosine recombinases, or DNA coupling proteins are required for cag T4SS-dependent DNA translocation. Analysis of the H. pylori 26695 genome identified several genes encoding orthologous T4SS components harbored within incomplete tfs3/tfs4 plasticity zones, including a TraG/VirD4-like DNA coupling protein (tfs4-encoded hp1006), XerD family tyrosine recombinases (tfs3-encoded hp0995 and tfs4-encoded hp1009), VirD2-like endonucleases (tfs3-encoded hp0996 FIG. 13D and truncated tfs4-encoded hp1004), and orphaned VirB11-like (hp1421) and VirB4-like (hp0441) ATPases (Fischer et al., 2020). While DNA recombinases, DNA coupling proteins, the truncated VirD2 ortholog Hp1004, and orphaned T4SS-associated ATPases were dispensable for TLR9 stimulation, the VirD2-like endonuclease Hp0996 was required for robust TLR9 activation (FIG. 13E). Comparison of Hp0996 to the canonical A. tumefaciens VirD2 relaxase enzyme revealed the presence of a conserved N-terminal relaxase domain and a C-terminal domain of unknown function (DUF) lacking the nuclear localization signal typically localized to the VirD2 C-terminus (FIG. 13D). While TLR9 activation phenotypes of the hp0996 isogenic strain were restored when Hp0996-HA was produced from a secondary chromosomal locus, complementation with either the Hp0996 N-terminus or C-terminus alone failed to restore DNA translocation phenotypes (FIG. 13E) suggesting that both protein domains are required for Hp0996 function.

VirD2 endonucleases bind the oriT of plasmid DNA, cleave the phosphodiester bond within the nicking region, and covalently attach to the 5′ end of the processed ssDNA for transport into plant cells (Christie et al., 2005). In contrast to canonical A. tumefaciens vir T4SS-mediated trans-kingdom delivery of plasmid-derived ssDNA, our data suggest that the cag T4SS translocates dsDNA originating from the chromosome into epithelial cells (FIG. 13A-C). To test the hypothesis that Hp0996 is a VirD2 ortholog that recognizes and cleaves cognate tfs3 oriT-like regions (FIG. 13F, inset; sequence identified in (Grove et al., 2013)), we expressed and purified recombinant maltose-binding protein (MBP) fused to H. pylori 26695 Hp0996 (MBP-Hp0996) for biochemical analyses. Recombinant Hp0996 strongly bound both ssDNA and dsDNA (FIG. 13F) harboring the tfs3 oriT-like sequence. While previous work characterized the enzymatic function of VirD2 N-terminal relaxase domains, the function of the VirD2 C-terminal region (DUF) has not been elucidated. We therefore analyzed the capacity of Hp0996^{NTD} and Hp0996^{CTD} to bind target DNA. While Hp0996^{NTD} and Hp0996^{CTD} bound ssDNA containing the tfs3 oriT sequence, only Hp0996^{CTD} tightly bound tfs3 oriT sequences in dsDNA (FIG. 13F), suggesting that the C-terminal DUF region anchors VirD2 to target DNA substrates. Consistent with the predicted endonuclease function, recombinant full-length Hp0996 and the Hp0996 N-terminal relaxase domain, but not the Hp0996 C-terminal DNA binding domain, exhibited cleavage of both ssDNA and dsDNA (FIG. 13G). To test the hypothesis that Hp0996 exhibits strand-specific relaxase activity, we next performed nicking and DNA linearization assays using plasmids containing a single tfs3 oriT-like region. Incubation of purified full length recombinant Hp0996 or Hp0996^{NTD}, but not Hp0996^{CTD}, resulted in a concentration-dependent conversion of supercoiled plasmid DNA to nicked open circle and linearized species (FIG. 13H), confirming that in the absence of other factors, Hp0996 can bind and catalyze tfs3 oriT-dependent DNA nicking and cleavage reactions. Given that the tfs3 oriT-like hexanucleotide 5′-ATCCTG-3′ motif appears approximately 376 times throughout the H. pylori 26695 chromosome, we propose a model whereby Hp0996 mediates cleavage of excess DNA fragments arising from overlapping or aborted rounds of chromosomal replication.

The A. tumefaciens VirD2 delivers plasmid-derived ssDNA into plant cells via translocation through the vir T4SS (Christie et al., 2005). We therefore investigated whether Hp0996 interacts with the cag T4SS apparatus to couple transfer DNA to the secretion channel. Bacterial two-hybrid protein-protein interaction analysis revealed that in comparison to negative (empty vector) and positive controls (Zip-Zip (Karimova et al., 2017), H. pylori DprA-DprA (Mortier-Barriere et al., 2007), and Cagα-Cagα (Savvides et al., 2003) interactions), Hp0996 strongly interacted with the VirB11-like ATPase Cagα, but not the predicted inner membrane component CagV, suggesting that Hp0996 recruits transfer DNA to the cytoplasmic face of the inner membrane cag T4SS ATPase complex for coupling to the secretion channel. Consistent with previous studies (Lin et al., 2020), Cagα, but not the VirD4-like coupling protein Cag5, was required for TLR9 activation phenotypes. Interactions between Hp0996 and portions of CagY and CagX that form the cag T4SS periplasmic ring structure (Sheedlo et al., 2020) were also detected by bacterial two-hybrid analysis (FIG. 13I-J), suggesting that similar to A. tumefaciens VirD2, Hp0996 potentially enters the translocation channel to mediate DNA delivery to gastric cells. Finally, transfer DNA immunoprecipitation assays demonstrate that in addition to the inner membrane-associated ATPases CagE and Cagα, Hp0996 is required for DNA loading into the cag T4SS core complex prior to encountering host cells (FIG. 13K), demonstrating a critical role of the VirD2-like endonuclease in effector DNA recruitment to the secretion channel. Collectively, these results identify a critical role of the tfs3-encoded relaxase in cag T4SS-mediated trans-kingdom conjugation and represent the first described example of endogenous T4SS chimerism in which orthologous component co-option expands machinery function and effector molecule repertoire.

### Discussion

STING-dependent immunosurveillance plays a critical role in maintaining gastric mucosal homeostasis and regulating inflammatory responses with carcinogenic potential (Ke et al., 2022). STING signaling has been implicated in the development and progression of stomach cancer, but whether STING activation promotes or restricts gastric carcinogenesis remains unresolved. For example, a previous study demonstrated that STING downregulation in primary gastric tumors was associated with increased malignancy and the progression of gastric cancer (Song et al., 2017), while a conflicting report determined that high STING expression in malignant tissues and tumor-associated macrophages was predictive of poor prognosis in stomach cancer patients (Miao et al., 2020). Thus, the molecular role of cGAS-STING signaling in chronic gastric inflammation and pre-malignant lesion development remains unresolved. Our study establishes a critical role of cag T4SS-mediated trans-kingdom DNA conjugation in stimulating STING-dependent outcomes that underscore infection-associated carcinogenesis.

In the context of bacterial infection, type I IFN responses have been associated with both protective and detrimental outcomes (Boxx and Cheng, 2016; Peignier and Parker, 2021). Previous work demonstrates that the H. pylori cag T4SS-dependent delivery of muropeptide fragments activates Nod1 sensing and IRF7-mediated type I IFN responses in epithelial cells that restrict bacterial proliferation via CXCL10-mediated immune cell recruitment (Viala et al., 2004; Watanabe et al., 2010). Our work demonstrates that during acute infection, CXCL10 is highly upregulated by cag T4SS activity in primary gastric epithelial cells (FIG. 9). In support of our model, recent work demonstrates that H. pylori counteracts type I IFN responses to control excessive inflammation via cag T4SS-independent mechanisms (Dooyema et al., 2022). In addition to increased transcript levels associated with interferon stimulated genes, we observed augmented production of transcripts associated with SMHD1 [a suppressor of type I IFN and NF-KB signaling (Chen et al., 2018)], IFI35 [a negative regulator of RIG-I signaling (Das et al., 2014)], and ISG15 [an IFN-α/13-inducible ubiquitin-like modifier that is a key negative regulator of IFN-α/13 immunity (Zhang et al., 2015)] in WT-infected primary gastric cells, suggesting that H. pylori has evolved mechanisms to dampen nucleic acid signaling pathways induced by cag T4SS activity (Dooyema et al., 2022). Consistent with this hypothesis, pro-tumorigenic immune effectors that suppress STING activation, including TRIM5 and TRIM22 (Dooyema et al., 2022), were significantly upregulated in WT-infected primary gastric epithelial cells (FIG. 9A-B). We speculate that similar to evasion strategies employed by numerous viruses, H. pylori may preclude nucleic acid reconnaissance via the cag T4SS-dependent translocation of as yet unidentified, evolutionarily-conserved protein effectors that target and neutralize components of DNA-sensing or STING signaling pathways. Alternatively, pro-inflammatory STING signaling may balance immunosuppressive responses stimulated by cag T4SS-dependent TLR9 activation (Varga et al., 2016a) as a mechanism to sustain gastric homeostasis during acute colonization.

Epithelial DNA damage, genomic alterations, and chromosomal instability are hallmarks of H. pylori-induced gastric cancer (Chaturvedi et al., 2014). While H. pylori cag T4SS activity has been shown to induce the formation of both double-stranded DNA breaks (DSBs) and micronuclei that potentially contribute to cGAS-STING signaling in gastric epithelial cells (Bauer et al., 2020; Hanada et al., 2014; Koeppel et al., 2015), our data suggest that translocated chromosomal DNA fragments serve as the predominant cytosolic cGAS trigger in the context of H. pylori infection (FIG. 7). In support of our data indicating that translocated chromosomal fragments are the key agonist that alerts cytosolic nucleic acid surveillance, we found that H. pylori effector DNA is packaged into exosomes released by gastric epithelial cells to enable paracrine-like signal amplification (FIG. 10). Consistent with a previous study demonstrating that intracellular Listeria monocytogenes infection stimulates the production of extracellular vesicles with IFN-inducing potential (Nandakumar et al., 2019), the delivery of pathogen-derived DNA via exosome release may represent a conserved mechanism by which epithelial cells potentiate nucleic acid surveillance-dependent danger signaling to uninfected tissues. We propose that H. pylori has evolved to exploit this innate defense mechanism to shape a tolerogenic immune response that favors persistent gastric colonization (Dooyema et al., 2022; Varga et al., 2016a).

While the exact mechanism by which effector DNA is precisely excised and shuttled through the cag T4SS while maintaining faithful genome inheritance is incompletely defined, our work represents a major advance in understanding how a single T4SS nanomachine can deliver a diverse effector repertoire to target host cells. Mechanistically, our data demonstrate that cag T4SS-dependent DNA delivery is coupled to chromosomal replication and replichore decatenation (FIG. 11 and FIG. 12). Similar to Neisseria gonorrhoeae T4 SS-dependent single-stranded chromosomal DNA export mechanisms (Callaghan et al., 2017), chromosomal partitioning influences cag T4SS-mediated DNA translocation. Indeed, our studies reveal that effector DNA coupling to the cag T4SS apparatus requires both FtsK membrane tethering and DNA translocase activity (FIG. 12). In support of the critical role of replichore decatenation in cag T4SS-dependent DNA translocation, H. pylori lacking the DNA recombinase XerH, which requires direct interaction with FtsK to resolve entangled dimers and catenated chromosomes (Bebel et al., 2016; Debowski et al., 2012), exhibited significantly reduced levels of TLR9 stimulation. Our studies suggest that while xerH mutants harbor increased DNA content per cell compared to the parental WT strain (Debowski et al., 2012), cag T4SS DNA translocation phenotypes are significantly impaired when chromosomal DNA topological isomers cannot be efficiently resolved (FIG. 12). Additionally, our findings establish that DNA effector molecules are pre-loaded into the cag T4SS apparatus to establish a ‘ready-to-fire’ nanomachine that can be rapidly deployed during acute infection and demonstrate that FtsK is required for partitioning DNA substrates into the cag T4SS (FIG. 12).

In contrast to a previous report (Tegtmeyer et al., 2022), our unexpected discovery that the cag T4SS evolved to usurp the tfs3-encoded VirD2-like relaxase to implement trans-kingdom conjugation (FIG. 13) addresses a previously unresolved paradox surrounding the mechanism by which H. pylori delivers chromosomal DNA to host cells. However, whether additional yet unidentified DNA coupling factors or remnant endonucleases facilitate relaxosome tethering to the cag T4SS apparatus remains unknown. We postulate that novel DNA binding proteins may aid in relaxosome recruitment to the cytoplasmic cag T4SS ATPase complex and that additional processing steps, occurring perhaps in the periplasm or at the bacterial cell surface, may be required for transfer DNA modification prior to delivery to host cells. Additionally, our finding that chromosomally-derived cag T4SS effector DNA is at least partially double-stranded diverges from canonical DNA conjugation models in which plasmid-borne ssDNA is translocated into target cells in complex with cognate relaxases. We speculate that this innovation represents yet another example of the remarkable apparatus flexibility afforded by orthologous component co-option.

Furthermore, our biochemical assays reveal that the Hp0996 C-terminal DUF serves as a DNA binding domain that strongly anchors the relaxase to both single- and double-stranded targets (FIG. 13F), revealing a previously unresolved function of VirD2 endonuclease C-terminal architectures. Consistent with a proposed gating mechanism controlling effector substrate switching at the inner membrane (Savvides et al., 2003), we demonstrate a direct interaction between Cagα and Hp0996 (FIG. 131-J) suggesting that Hp0996 docking may trigger a conformational shift that biases cag T4SS activity towards trans-kingdom conjugation. Furthermore, we detected direct interactions between Hp0996 and portions of CagY and CagX comprising the cag T4SS periplasmic ring complex via bacterial two-hybrid assays (FIG. 13I-J), suggesting that Hp0996 enters the periplasm complexed with transfer DNA. Consistent with the observation that DNA translocation phenotypes are susceptible to exogenous nucleases and DNA cross-reactive antibodies (FIG. 13A-C), our data point towards a novel two-step secretion mechanism whereby effector DNA is transported across the bacterial outer membrane prior to entering host cells. Thus, DNA does not appear to be secreted through a potential cag T4SS-associated pilus conduit as suggested for archetypical conjugation systems.

Our results provide the first direct evidence that H. pylori delivers immunostimulatory chromosomal DNA fragments into target epithelial cells and demonstrate that translocated effector DNA physically binds and activates cytosolic DNA-sensing reconnaissance systems. The observation that chromosomal fragments encompassing full-length eukaryotic genes can be excised and directed to the cag T4SS for delivery into target cells underscores the potential to exploit H. pylori trans-kingdom conjugation for designer DNA cargo delivery. Notably, our work provides the essential experimental framework required to harness the cag T4SS as a mucosa-targeted DNA vaccine or therapeutic delivery device that can be pharmacologically controlled in vivo. Studies to determine the maximal DNA fragment length that can be efficiently transported to gastric cells via cag T4SS mechanisms are currently underway.

Finally, our work highlights the importance of understanding host-pathogen conflicts that engage cellular PRR signaling axes to drive chronic inflammation and stimulate the development of infection-related malignancies. While we propose that translocated microbial DNA plays a critical and underappreciated role in the development of inflammation-associated carcinogenesis, further investigations are required to understand STING-dependent outcomes within the context of gastric cancer. Our findings identify a central role of cag T4SS activity in eliciting cGAS-STING immunosurveillance in the gastric epithelium; however, additional studies are warranted to understand how STING activation shapes the gastric immune landscape to enable persistent H. pylori colonization and to elucidate whether STING signaling influences the development of pre-malignant lesions. Finally, our work more broadly points towards therapeutic STING modulation as a promising intervention strategy to reduce the incidence and severity of infection-associated malignancies that arise as a consequence of chronic inflammation.

### Architectural Asymmetry Enables DNA Transport Through the Helicobacter pylori Cag Type IV Secretion System

Architectural symmetry mismatch is a remarkable evolutionary innovation exhibited by diverse bacterial nanomachines including the dynamic type IV secretion system (T4SS) superfamily¹⁻³. Recent advances in the structural definition of paradigmatic T4SS machineries uncovered unexpected asymmetric features at the junction between the inner membrane complex (IMC) and outer membrane complex (OMC), or between concentric rings comprising the OMC in expanded T4SS architectures¹⁻⁴. In addition to T4SS machineries, symmetry mismatch has been observed in other bacterial secretion systems including the T2SS⁵, T3SS^{6,7}, and T6SS⁸, suggesting an important, yet unresolved, functional significance. Although the structural relationships between T4SS apparatus components and cognate effector substrates transiting the secretion channel are currently unresolved, sub-structure symmetry mismatch presumably provides conformational mobility to facilitate the unidirectional ratcheting of selected cargo between adjacent architectural layers. Accordingly, asymmetry between structural sub-complexes may impart the incredible functional plasticity and apparatus dynamism required to orchestrate diverse substrate selection and enable directional effector translocation^{1,3,9}.

The versatile T4SS nanomachine is employed by Gram-negative and Gram-positive organisms to deliver DNA, proteins, or other substrates to target prokaryotic or eukaryotic cells. In Gram-negative organisms, ‘minimized’ T4SS machineries encompass roughly 12 conserved components, denoted VirB1 through VirB11 and VirD4, following a standardized nomenclature derived from the prototypical Agrobacterium tumefaciens vir T4SS¹⁰⁻¹². In these streamlined configurations, the apparatus IMC subassembly forms part of the translocation channel that spans the periplasm and couples to the OMC through a stalk-like structure^{9,13-15}. In some architectures, elongated VirB10 domains act as connectors bridging the OMC and IMC^{9,11,15}. Multiple ATPases, including the VirD4 coupling protein that delivers nucleoprotein complexes to the secretion channel^{11,16-19}, power cargo translocation across the bacterial envelope.

Trans-kingdom DNA transfer achieved by minimized T4SS machineries is an evolutionary remnant of generalized interbacterial conjugation mechanisms^{10,20,21}. Thus far, relatively few ‘dual function’ systems that deliver nucleoprotein complexes across kingdom boundaries have been identified. The prototypical dual function vir T4SS harbored by A. tumefaciens translocates tumor-inducing DNA (T-DNA) and associated effector proteins into recipient plant cells in a contact-dependent manner^{10,20,22-25}. Seminal work using a transfer DNA immunoprecipitation (TrIP) assay²⁴ to trace the DNA translocation pathway through the vir T4SS secretion channel provided a mechanistic understanding of how apparatus components coordinate nucleoprotein delivery across the bacterial outer membrane. In addition to A. tumefaciens, recent studies demonstrate that dual purpose and expanded machineries in Rhizobium etli²⁶, Bartonella henselae²⁷⁻³⁰, Coxiella burnetii²⁹, Legionella pneumophila^{29,31,32}, and Helicobacter pylori³³ have the fundamental capacity to translocate DNA into eukaryotic cells via T4SS-dependent mechanisms.

Structural asymmetry within secretion system architecture is fundamentally important for apparatus diversification and biological function. However, the mechanism by which symmetry mismatch contributes to nanomachine assembly and interkingdom effector translocation are undefined. Here, we show that architectural asymmetry orchestrates dynamic substrate selection and enables trans-kingdom DNA conjugation through the Helicobacter pylori cag type IV secretion system (cag T4SS). Structural analyses of asymmetric units within the cag T4SS periplasmic ring complex (PRC) revealed intermolecular pi-pi stacking interactions that coordinate DNA binding and license trans-kingdom conjugation without disrupting the translocation of protein and peptidoglycan effector molecules. Additionally, we identified a novel proximal translocation channel gating mechanism that regulates cargo loading and governs substrate transport across the outer membrane. We thus propose a model whereby the organization and geometry of architectural symmetry mismatch exposes pi-pi interfaces within the PRC to facilitate DNA transit through the cag T4SS translocation channel.

We employed a structure-function approach to delineate mechanisms underscoring cag T4SS-dependent DNA translocation. We report the identification of a novel cag T4SS gating mechanism that regulates the passage of DNA and protein effectors across the outer membrane and licenses the import of exogenous molecules into the periplasm. We provide evidence that in addition to direct interactions with components of the cag T4SS IMC and PRC, the VirB2-like pilin CagC polymerizes to form an ‘endopilus’ gate or plug domain that controls effector transport through the secretion channel. We propose a model in which CagC polymers dynamically assemble into an endopilus-like structure corresponding to the stalk region that bridges the IMC and periplasmic complexes in the cag T4SS machinery. Mechanistically, we demonstrate that the VirB9 ortholog CagX facilitates secondary substrate selection within the PRC via dsDNA-pi-stacked interactions at sites exhibiting architectural asymmetry. Whereas disruption of intermolecular CagX pi-pi stacking resulted in a significant reduction of DNA binding in vitro and diminished trans-kingdom conjugation in vivo, non-nucleic acid cargo was effectively delivered into gastric epithelial cells when PRC pi-stacking interactions were genetically ablated. Collectively, these studies highlight architectural innovations underpinning substrate selectivity and provide a mechanistic framework for understanding how the remarkable cag T4SS nanomachine coordinates diverse effector molecule delivery into gastric epithelial cells.

### Results

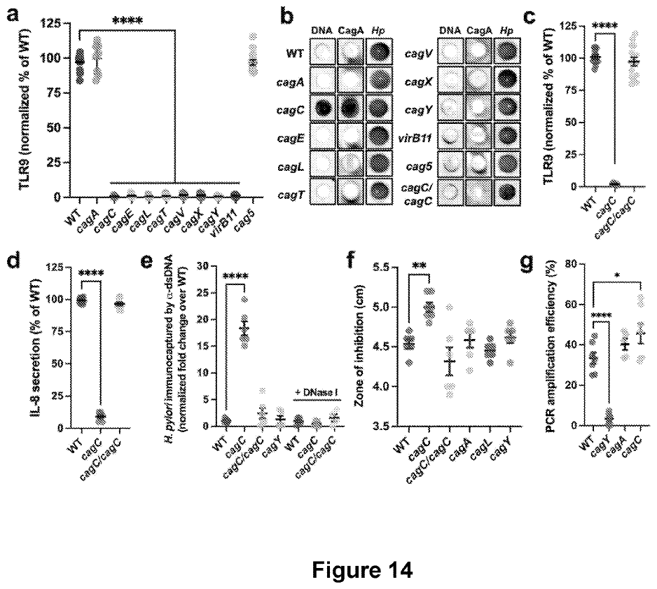

CagC gates effector translocation through the cag T4SS. The cag T4SS harbors multiple vir T4SS homologs including CagX, CagY, CagT, CagV, CagE, Cagα, CagL, CagC, and Cag5 (VirB9, VirB10, VirB7, VirB8, VirB4, VirB11, VirB5, VirB2, and VirD4, respectively)^{1,3,17,18,22,38,42-44}. Prior work identified ‘uncoupling’ mutations within the A. tumefaciens vir T4SS that selectively disarm T-pilus biogenesis without perturbing trans-kingdom conjugation^{12,45,46} Similarly, various H. pylori cag T4SS-dependent phenotypes, including CagA translocation and IL-8 stimulation, occur via uncoupled mechanisms that require incongruent subsets of cag T4SS components^{18,34,47,48}, raising the hypothesis that molecularly dissimilar cag T4SS substrates are secreted into host cells via distinct mechanisms. To test this hypothesis, we first analyzed the contribution of vir T4SS homologs in TLR9 stimulation, an outcome that requires a functional cag T4SS^{18,33,34,49}. Consistent with previous studies analyzing cag T4SS-dependent phenotypes^{18,35,43,49}, TLR9 stimulation occurred in a CagA-independent manner^{33,34} and required recognized vir T4SS homologs, with the exception of the VirD4-like coupling protein Cag5¹⁸, suggesting that DNA trafficking through the secretion channel occurs via non-canonical trans-kingdom conjugation (FIG. 14a). To investigate the mechanism by which disparate cag T4SS substrates are selected for transport to target host cells, we next analyzed potential structural features that could facilitate DNA substrate selection for export through the cag T4SS apparatus. Previous work identified a single point mutation within the A. tumefaciens vir T4SS OMC that confers secretion channel gating defects resulting in the uncontrolled release of VirE2 effectors to the bacterial cell surface⁵⁰. We reasoned that the cag T4SS may harbor a similar gating mechanism based on the presence of multiple vir T4SS homologs localized throughout the apparatus architecture^{1,3,18,38}. To test whether individual cag T4SS components are associated with apparatus gating, we employed non-denaturing colony blot assays developed to interrogate translocation channel integrity in A. tumefaciens⁵⁰. In contrast to WT H. pylori, which does not release detectable levels of cag T4SS effector molecules into the extracellular milieu, isogenic mutants deficient in cagC accumulated surface-exposed CagA and transfer DNA in a host cell contact-independent manner (FIG. 14b). Genetic complementation of the cagC isogenic mutant at a heterologous chromosomal locus abrogated effector leakage (FIG. 14b) and restored cag T4SS-dependent TLR9 activation (FIG. 14c) and IL-8 stimulation phenotypes (FIG. 14d), suggesting that CagC regulates channel gating or substrate passage across the outer membrane. In support of the hypothesis that CagC forms a specialized plug or gating mechanism, inactivation of other reported structural (CagX, CagY, CagT, CagV, CagL) or energetic (CagE, Cagα, Cag5) components did not affect cag T4SS translocation channel integrity (FIG. 14b).

To quantitatively monitor the uncontrolled release of effector DNA into the extracellular milieu, we developed a live cell assay to enumerate H. pylori that accumulate surface-exposed transfer DNA in the absence of host cell contact. For these studies, WT H. pylori or the indicated isogenic mutant strain was incubated with anti-double-stranded DNA (anti-dsDNA) monoclonal antibodies or an equivalent volume of a corresponding IgG isotype control, and immunocaptured bacteria were isolated by Protein G-conjugated magnetic bead separation. Compared to WT and the cagY strain, which does not assemble the cag T4SS core complex^{1,3,38} and thus does not exhibit effector leakage (FIG. 14b), significantly more cagC bacteria were immunocaptured by anti-dsDNA antibodies (FIG. 14e). Consistent with a role in effector gating, genetic complementation of the cagC mutant restored immunocaptured bacteria to WT levels (FIG. 14e). Likewise, incubating bacterial cultures with DNase I prior to anti-dsDNA immunocapture markedly reduced levels of isolated H. pylori cagC (FIG. 14e) demonstrating that aberrantly localized dsDNA is tethered to the bacterial cell surface.

We next sought to determine whether cag T4SS pore gating defects afford unrestricted access to the periplasm. We reasoned that loss of either secretion channel integrity or pore gating would license the influx of bulky molecules, such as erythromycin, that are typically impermeable to the Gram-negative outer membrane⁵⁰⁻⁵². To test whether potential cag T4SS pore gating mechanisms control periplasm access, we employed erythromycin sensitivity assays to detect changes in outer membrane permeability⁵¹. Compared to WT and isogenic mutants that do not exhibit effector molecule leakage, the cagC strain displayed significantly increased pore permeability (FIG. 14f) indicating that in the absence of host cell contact, CagC is important for stabilizing the secretion channel in a tightly closed conformation. Erythromycin susceptibility patterns of the genetically complemented cagC mutant phenocopied the parental WT strain (FIG. 14f), further demonstrating that CagC controls pore gating and access to the periplasm via leaky secretion channels.

Previous work determined that effector DNA is pre-loaded into the cag T4SS apparatus prior to initiating host cell contact to generate a ‘ready-to-fire’ nanomachine³⁴. To test whether defective gating modulates DNA loading, we used a modified transfer DNA immunoprecipitation assay developed in A. tumefaciens²⁴ to monitor levels of DNA confined within the cag T4SS core complex lumen³⁴. For these studies, we isolated cag T4SS assemblies from chemically cross-linked H. pylori via immunopurification targeting CagY (a constituent of the cag T4SS outer membrane-associated complex and periplasmic ring structure^{1,3,38}), and monitored DNA loading by PCR analysis of co-purifying chromosomal fragments that remained shielded from DNase degradation within the secretion channel. In agreement with a previous report³⁴, DNA fragments were readily amplified from cross-linked CagY preparations obtained from WT and cagA-deficient H. pylori, but not from mock preparations generated from the cagY isogenic strain (FIG. 14g). Consistent with a role in regulating DNA passage through the cag T4SS OMC, significantly more DNA co-purified with CagY complexes in strains lacking cagC (FIG. 14g), demonstrating that CagC is not required for effector DNA loading into the distal secretion channel. Collectively, these studies demonstrate that loss of CagC confers cag T4SS channel leakiness that enables the unrestricted export of effectors and the import of exogenous small molecules into the periplasm.

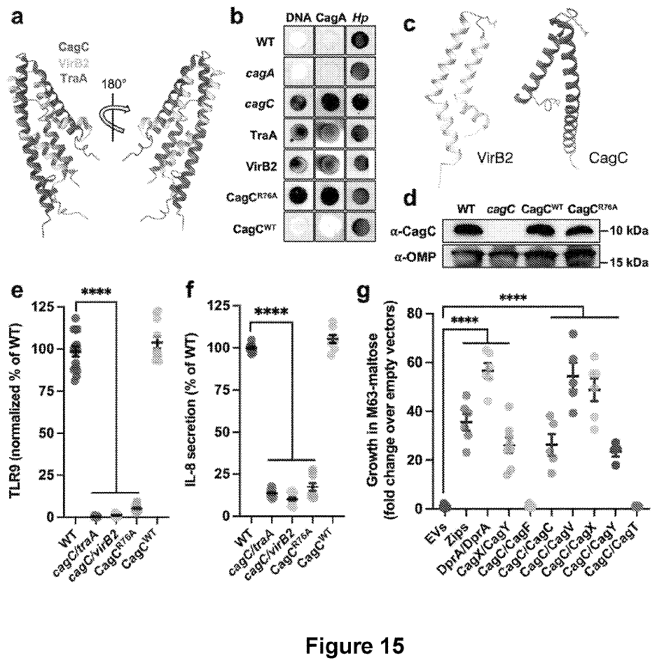

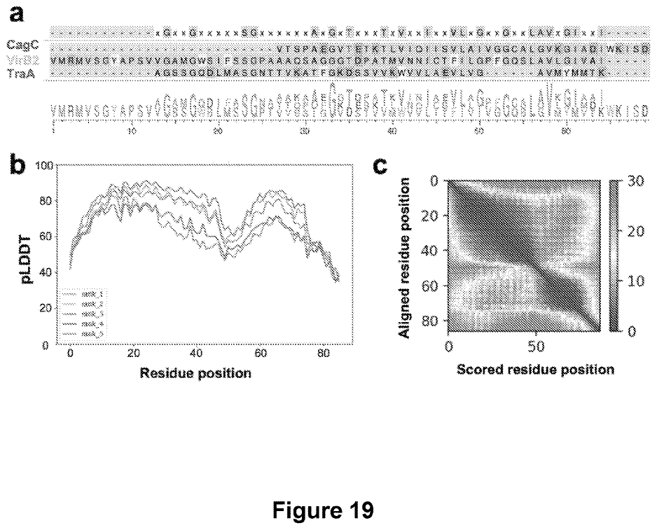

CagC is a specialized T4SS pilin ortholog. We next performed protein sequence analysis and structural modeling to further investigate the role of CagC as a novel gating mechanism within the cag T4SS apparatus. In accordance with a previous study analyzing pilin-like motifs in bacterial cell surface-exposed polypeptides⁵³, CagC exhibited primary sequence similarity to canonical T4SS pilins including A. tumefaciens VirB2 and E. coli TraA (FIG. 19a). AlphaFold2⁵⁴ modeling of the mature CagC monomer lacking the leader peptide⁵⁴⁻⁵⁶ revealed a predicted helical structure that closely resembles the experimentally resolved structures of conjugative pilins VirB2^{57,58} and TraA⁵⁹ (FIG. 15a and FIG. 19b,c). In contrast to a previous report suggesting that conjugative pilins are cyclic in nature⁶⁰, superimposition of predicted CagC structures with monomeric VirB2 and TraA revealed a slightly U-shaped protomer corresponding to structural conformations in which the free pilin termini were oriented towards the exterior of assembled canonical T-pilus or F-pilus filaments⁵⁷⁻⁵⁹ (FIG. 15a).

Based on the predicted structural conservation, we hypothesized that orthologous pilins could rescue cag T4SS defects exhibited by the cagC isogenic mutant. To these this hypothesis, we expressed either A. tumefaciens virB2 or E. coli traA in the cagC isogenic mutant and analyzed cag T4SS-dependent outcomes elicited by each chimera. Despite predicted structural similarities, genetic complementation of cagC with either virB2 or traA did not restore cag T4SS pore gating (FIG. 15b), TLR9 activation (FIG. 15e), or IL-8 stimulation (FIG. 15f) defects, suggesting that CagC fulfills a unique role within the cag T4SS. One possibility is that CagC forms an ‘endopilus’ within the secretion channel lumen corresponding to the stalk-like density observed in the in situ cag T4SS architecture^{17,37}. Recent studies analyzing VirB2 protomers identified arginine residues (Arg 91) that protrude into the T-pilus lumen and form extensive electrostatic interactions required for pilus assembly and structural stabilization^{57,58} (FIG. 15c). Congruent with a predicted function as a specialized pilin, CagC harbors a single arginine residue in the mature protomer (Arg 76) corresponding to VirB2 Arg 91 (FIG. 15c). We reasoned that similar to T-pilus biogenesis, polymerization of a putative ‘endopilus’ could be selectively disrupted by modifying positively charged residues within the predicted CagC luminal loop (FIG. 15a,c). To investigate whether Arg 76 is required for CagC function, we generated a CagC variant containing a R76A single point mutation expressed from a heterologous chromosomal locus in the cagC mutant. Western blot analysis of H. pylori whole cell lysates reveled high levels of CagC^{R76A} expression (FIG. 15d), demonstrating that in contrast to VirB2 Arg 91^{57,58}, CagC Arg 76 does not contribute to protomer stability. Consistent with a role as a putative ‘endopilus’ or gating mechanism, CagC^{R76A} exhibited uncontrolled DNA and effector protein release to the bacterial cell surface (FIG. 15b). Accordingly, compared to the parental WT strain and cagC mutants genetically complemented with CagC^{WT}, CagC^{R76A} variants were defective in TLR9 activation (FIG. 15e) and IL-8 stimulation (FIG. 15f), indicating that positively charged residues within the luminal loop are important for CagC protomer function or multimerization into higher order structures. Taken together, these data suggest that the specialized pilin CagC assembles into a novel plug or gate-like apparatus to control effector passage through the secretion system pore.

Periplasmic cag T4SS assemblies bind transfer DNA. We next sought to understand the mechanism by which CagC regulates effector transit through the translocation channel. In the mature form, CagC pilin subunits are approximately 9.2 kDa (FIGS. 15d) and attempts to generate a functional epitope-tagged fusion protein for immunopurification studies were unsuccessful. Therefore, we employed a bacterial two-hybrid (BTH) screening approach designed to characterize interactions among both soluble and membrane-associated components⁶¹⁻⁶³ to localize CagC complexes within the cag T4SS apparatus. Analysis of direct protein-protein interactions revealed that in comparison to negative (empty vector) and positive (Zip-Zip⁶² and H. pyloriDprA-DprA⁶⁴ ) controls, CagC strongly interacted with components predicted to comprise the T4SS apparatus inner membrane complex (IMC) including the bitopic VirB8 homolog CagV (FIG. 15g). BTH assays also revealed strong interactions among the CagX N-terminal domain (residues 41-310) and corresponding CagY fragments (residues 1469-1603, designated CagY_{CT}) that assemble into the periplasmic ring complex (PRC)^{1,3,38}, demonstrating the ability of our screening approach to detect direct protein-protein interactions occurring in the periplasm. In addition to CagC-CagV, we detected CagC-CagC protomer interactions (FIG. 15g), suggesting that CagC can self-polymerize into higher order structures or subcomplexes. Whereas interactions among CagC and components localized to either the cytoplasm (the chaperone-like molecule CagF⁶⁵) or the periphery of the cag T4SS outer membrane complex (the VirB7 homolog CagT^{1,3,38}) were not detected, CagC directly interacted with both CagX and CagY_{CT} (FIG. 15g). These observations led to the hypothesis that CagC subassemblies bridge the IMC and PRC.

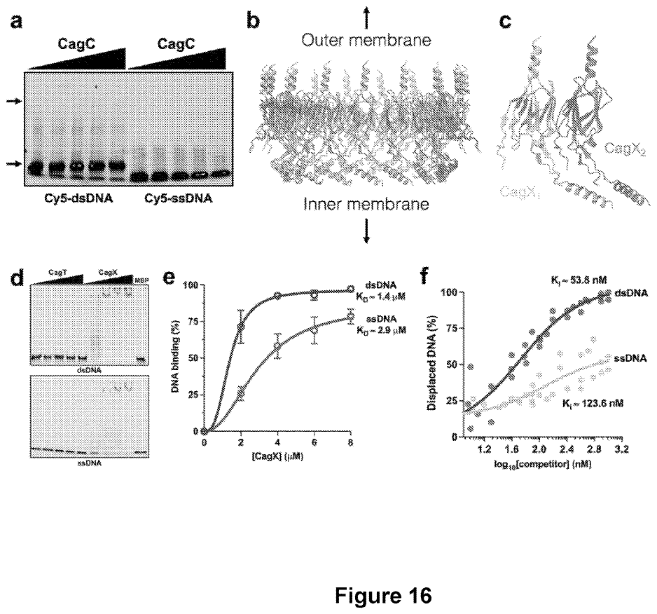

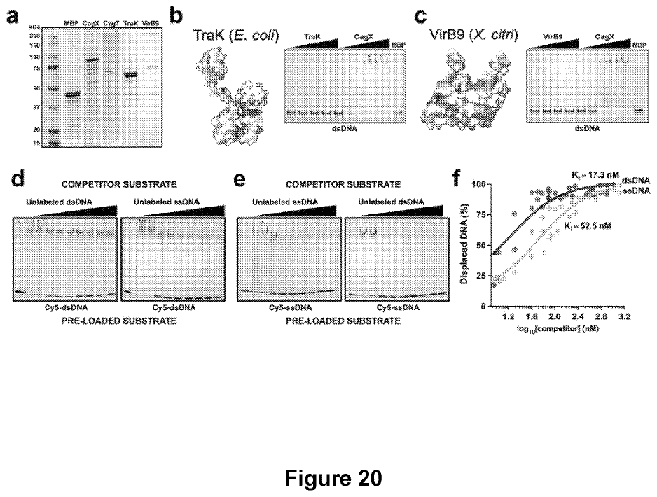

One possibility is that a putative CagC ‘endopilus’ assembly corresponding to the cag T4SS stalk-like region visualized in situ^{17,37} may dynamically assemble and retract to regulate effector DNA gating within the PRC via a piston-like mechanism. To test the hypothesis that CagC interacts with DNA, we purified recombinant CagC and performed electromobility shift assays (EMSA) to analyze DNA binding capacities. In agreement with a previous study suggesting that dsDNA is delivered to host cells in a cag T4SS-dependent manner³⁴, CagC weakly bound 58-mer effector dsDNA sequences³⁴, but not corresponding 58-mer ssDNA targets (FIG. 16a). Based on the observation that CagC interacts with both CagX and fragments of CagY that comprise the PRC (FIG. 15g and FIG. 16b,c), we questioned whether other cag T4SS subcomplexes bind DNA to facilitate cargo transfer. Compared to maltose-binding protein (MBP) or recombinant MBP-CagT controls, MBP-CagX robustly bound target H. pylori DNA sequences³⁴ in a concentration-dependent manner (FIG. 16d,e). In contrast to CagX, TraK (E. coli F plasmid) and VirB9 (A. tumefaciens vir T4SS) homologs harbored by conjugative DNA transfer systems did not interact with DNA (FIG. 20b,c), suggesting that DNA binding within the PRC is a unique structural innovation in the cag T4SS apparatus. Consistent with the observation that CagC displays preferential interactions with dsDNA substrates (FIG. 16a), recombinant CagX exhibited a stronger binding affinity for dsDNA compared to corresponding ssDNA sequences (FIG. 16e). In support of this result, DNA competition assays demonstrated CagX preferential binding to dsDNA (FIG. 16f and FIG. 20d-f). Together, these studies identify CagX as a novel periplasmic DNA-binding T4SS component.

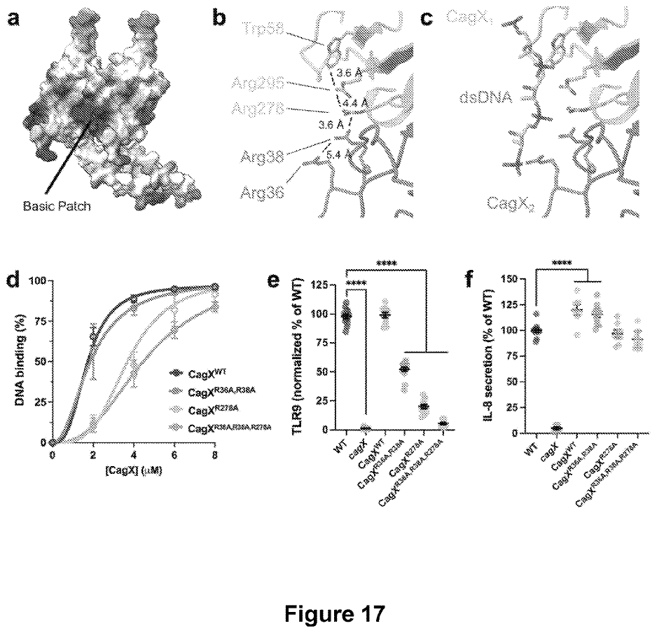

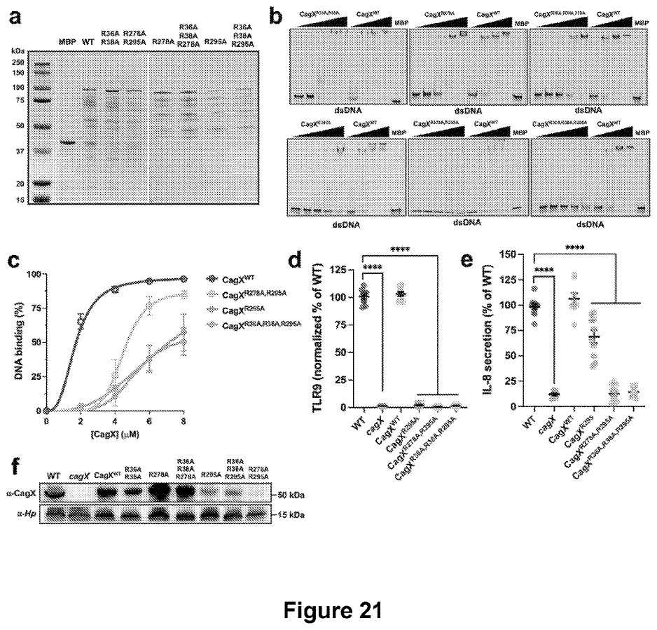

Symmetry mismatch enables DNA loading into the secretion channel. To delineate the molecular determinants of DNA binding within the cag T4SS PRC, we employed a structure-guided analysis to identify potential DNA binding sites in CagX-CagY assemblies. Close inspection of the CagX structure^{1,3} (FIG. 16c) revealed a basic patch formed at the interface of adjacent CagX subunits within the PRC (FIG. 17a) that represented a potential DNA binding region. We hypothesized that overlapping p (pi) orbitals among a series of arginine (Arg 36, 38, 278, and 295) and tryptophan (Trp 58) residues in the basic patch would produce electrostatic interfaces that enable DNA interactions within the secretion channel (FIG. 17b). Notably, the diameter and geometry of the proposed DNA binding site supports this assignment (FIG. 17c). To test the hypothesis that electrostatic CagX interfaces facilitate DNA loading into the secretion channel, we generated CagX variants by introducing point mutations in adjacent arginine residues within the basic patch region to disrupt intermolecular pi-stacking interactions (FIG. 17b). In comparison to CagX^{WT}, CagX variants in which Arg 36 and Arg 38 were replaced with alanine (CagX^{R36A,R38A}) exhibited similar dsDNA binding affinities in vitro, whereas CagX variants harboring the Arg 278 single point mutation (CagX^{R278A}) exhibited significantly decreased DNA binding capacities that were further reduced when consecutive Arg residues were disrupted in tandem (CagX^{R36A,R38A,R278A}) (FIG. 17d and FIG. 21a,b). Congruent with our hypothesis, H. pylori cagX mutants expressing CagX^{R36A,R38A}, CagX^{R278A}, or CagX^{R36A,R38A,R278A} translocated significantly less DNA into host cells, resulting in markedly reduced levels of TLR9 activation compared to the parental WT strain and cagX mutants genetically complemented by CagX^{WT} (FIG. 17e). Analysis of additional cag T4SS-dependent phenotypes produced by the CagX^{R36A,R38A,}, CagX^{R278A}, and CagX^{R36A,R38A,R278A} variant strains revealed that despite the observed DNA translocation defects, levels of IL-8 secretion were comparable to the WT and CagX^{WT} complemented strains (FIG. 17f), suggesting that DNA delivery is mechanistically uncoupled from the translocation of other cag T4SS substrates. In contrast, disrupting Arg 295via single point mutation or in tandem with Arg 36, Arg 38, or Arg 278 destabilized CagX, resulting in significantly decreased DNA binding in vitro (FIG. 21b, c) and markedly reduced cag T4SS activity (Supplementary FIG. 3d-f). We thus speculate that Arg 295 constitutes a critical CagY-binding interface in the PRC. Collectively, these studies indicate that intermolecular CagX pi-stacking interactions generate a DNA-binding pocket required for periplasmic substrate selection and trans-kingdom conjugation.

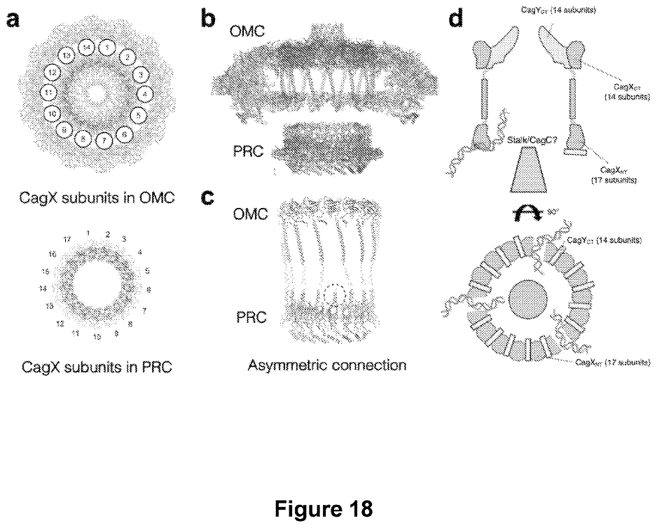

Analysis of cag T4SS architecture resolved by single particle reconstruction indicates that portions of CagX and CagY assemble into structures localized to both the OMC and the PRC (FIG. 18), producing a striking symmetry mismatch^{1,3}. In the current study, we identified a putative DNA binding site on CagX that is centered around resides Arg 36, 38, 278, 295 and Trp 58. The high-resolution cryo-EM structure indicates that this site is occupied by the periplasmic portion of CagY and thus, DNA binding may be excluded in the assembled PRC³. However, we note that the cryo-EM structure was determined through the imposition of symmetry and argue that this map omits asymmetric features. To that end, a low-resolution reconstruction of the PRC detected 14 copies of CagX in the OMC and 17 copies of CagX in the PRC (FIG. 18a) with 14 tube-like helical densities bridging the OMC (CagX C-terminus) and the PRC (CagX N-terminus)³ (FIG. 18b,c). This observation suggests that the additional copies of CagX in the PRC provide DNA interaction sites to facilitate nucleic acid trafficking through the core complex (FIG. 18d). We thus propose a model whereby asymmetric regions of the PRC assemble to form a DNA binding site for substrate selection and loading into the cag T4SS distal secretion channel (FIG. 18d).

### Discussion

Trans-kingdom DNA conjugation is an ancient mechanism that profoundly impacts host cell biology. The identification of DNA substrate selection mechanisms within the cag T4SS PRC highlights novel evolutionary adaptations that enable expanded machinery function. As previously suggested for the A. tumefaciens vir T4SS⁶⁶, the VirB9 ortholog CagX exhibits N-terminal domains that contribute to protein stability and establish inter-subunit contacts required for DNA substrate selection and trafficking through the translocation channel. Notably, our study uncovered multiple ‘uncoupling’ mutations that specifically disrupt DNA translocation without perturbing delivery of other substrates into host cells, highlighting remarkable cag T4SS apparatus flexibility. The capacity of CagX to recognize DNA and mediate selective substrate transfer through the cag T4SS OMC provides intriguing evidence suggesting that in addition to prototypical coupling proteins, periplasmic architectural features recruit effector cargo to the secretion channel. We speculate that CagX architectural asymmetry thus represents a secondary substrate selection checkpoint that regulates cargo transit through the periplasm and across the distal OMC translocation channel. We hypothesize that symmetry mismatch in other dual function T4SS machineries, and potentially other diverse secretion system architectures, similarly orchestrates substrate selection to ensure spatiotemporal and unidirectional translocation into target cells. Collectively, these observations expand the growing collection of disengaged systems exhibiting selective substrate translocation^{34,47} through evolutionarily divergent T4SS nanomachines in which scaffold biogenesis is uncoupled from trans-kingdom DNA conjugation^{50,66,67}.

Our studies to define the architectural determinants of cag T4SS substrate selection revealed a novel CagC-dependent gating mechanism that regulates cargo transfer through the proximal translocation channel into the PRC (FIGS. 14,15). Consistent with a proposed role as an endopilus-like structure, previous work demonstrates that CagC is a membrane-associated VirB2 ortholog that is localized to the bacterial cell surface via a process that requires cag T4SS apparatus biogenesis⁵³. Additional studies report the assembly of filamentous structures at the H. pylori-gastric epithelial cell interface that were proposed to constitute an extracellular cag T4SS-associated appendage analogous to interbacterial conjugative pili or the A. tumefaciens T pilus^{48,53,68-70}. However, the molecular composition of these structures has not been elucidated and multiple reports have identified several inconsistencies that argue against the presence of a canonical cag T4SS conjugative pilus. For example, H. pylori isogenic mutants deficient in the OMC component CagY, which forms the central translocation channel and is required for core complex assembly^{1,3,4,38}, produce extracellular filaments at the same level as WT bacteria^{68,69}. Thus, it is difficult to envision how a cag T4SS-associated pilus would nucleate and polymerize in the absence of (i) a translocation channel required for pilin transport to the bacterial cell surface and (ii) the OMC platform on which the conjugative pilus assembles⁷¹. While a putative cag T4SS-associated pilus may nucleate in the periplasm or at the inner membrane complex via mechanisms similar to T3SS needle biogenesis⁷², evidence strongly supports a model whereby prototypical T4SS conjugative pilus structures polymerize at the outer membrane⁷¹.

Furthermore, extracellular cag T4SS-associated filaments have not been observed in frozen hydrated H. pylori-gastric cell co-cultures imaged via cryo-electron tomography (cryo-ET) under near native conditions^{17,37}. While a conjugative pilus was not directly observed in the current study, our results provide compelling evidence for the assignment of CagC as a VirB2 pilin structural ortholog that forms a gating mechanism within the proximal secretion channel. Similar to the conjugative F pilus and the vir T4SS T pilus, we propose that CagC protomers assemble into a pentameric endopilus-like structure that lines the secretion channel. Assembled CagC pentamers may serve as a scaffold for biogenesis of heteromeric complexes that incorporate the proposed VirB5-like minor pilin CagL^{70,73,74} to form the cone-shaped stalk density that bridges the IMC and PRC^{17,37}. Stalk-like structures with similar sizes and geometries have been identified in cryo-ET atomic models of other T4SS machineries including the R388 conjugative system⁹ and the L. pneumophila dot/icm T4SS 75-77. Alternatively, CagC may form the rod structure observed spanning the periplasm and originating from inner membrane platforms within the cag T4SS architecture in situ³⁷. Finally, in conjunction with the observation that H. pylori deficient in cagC produce filamentous structures at the microbe-epithelial cell interface⁶⁹, the discovery of outer membrane-derived nanotubes produced by H. pylori in both the presence and absence of host cell contact^{37,78} support the hypothesis that the intercellular structures visualized in scanning electron microscopy studies are bacterial cell surface appendages unrelated to the cag T4SS.

H. pylori trans-kingdom conjugation occurs via incompletely defined mechanisms that require evolutionarily divergent VirD2-like relaxases and the DNA translocase/partitioning protein FtsK for nucleic acid cargo coupling to the translocation channel³⁴. Our work points towards a novel two-step substrate translocation mechanism that involves secondary DNA substrate selection within asymmetric CagX subunits in the PRC (FIG. 18). In this model, membrane tethered FtsK may form a pore in the inner membrane to pump effector DNA into the periplasmic space for substrate selection by the cag T4SS PRC. Alternatively, FtsK may act as a modified coupling protein that displaces cognate Cag5 from the cag T4SS cytoplasmic apparatus to deliver DNA directly into the proximal secretion channel. In this scenario, the stalk-like structure may dynamically retract and extend to ensure efficient DNA loading into the distal translocation channel. Secondary DNA substrate selection within the PRC chamber would therefore safeguard unidirectional translocation into host cells. This observation is strikingly similar to the previously suggested models of DNA trafficking through the A. tumefaciens vir T4SS whereby VirB9 C-terminal domains presumably interact with cellular VirB2 pilin subunits to stabilize the secretion channel^{24,66}. Likewise, the identification of VirB10-dependent gating mechanisms within the interior of the vir T4SS OMC chamber⁵⁰ and single point mutations in CagC that license the uncontrolled release of cag T4SS substrates to the bacterial cell surface (FIGS. 14,15) suggest that translocation channel gating is a fundamental mechanism within diverse dual function T4SS architectures. In the context of previously identified vir T4SS ‘uncoupling’ mutations that enable DNA translocation in the absence of extracellular pilus biogenesis, and evidence demonstrating that cag T4SS-dependent DNA transfer is susceptible to exogenous nucleases and neutralizing DNA antibodies^{33,34}, we speculate that CagC forms an internal endopilus that directs DNA through the distal secretion channel. We propose that while CagC is not required for DNA loading into the proximal translocation channel (FIG. 14g), putative endopilus-like structures may trigger conformational changes that stabilize the OMC pore. Subsequently, CagC polymers may line the translocation channel lumen to form a tight mating junction spanning the cellular interface. As CagC is required for the delivery of all identified cag T4SS substrates across the host cell membrane^{53,69} (FIG. 14), a putative CagC endopilus may function as a target cell sensor that locks the OMC pore into a translocation-competent state or as a signal transducer that propagates external cues to stimulate substrate switching. Coupled to secondary substrate selection mechanisms within the cag T4SS PRC, these studies broaden our understanding of how nanomachine architectural asymmetry orchestrates the release of diverse molecular cargo across the bacterial outer membrane.

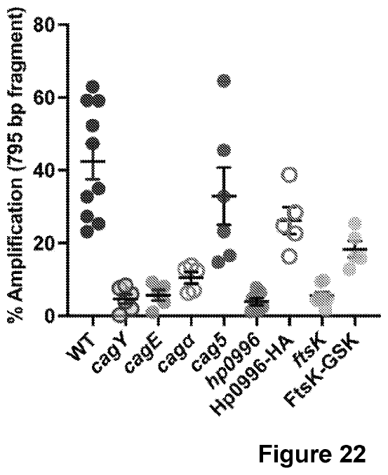

Future work to decipher complex structural relationships within the assembled secretion channel and periplasmic apparatus will undoubtedly provide mechanistic details underscoring interkingdom cargo transport through evolutionarily diverse machineries. For example, the present investigators have found that transfer DNA loading into the H. pylori cag T4SS apparatus in the absence of host cell contact requires the structural component CagY, Hp0996, FtsK, and the cag T4SS ATPases CagE and Caga, but not the VirD4-like coupling protein Cag5 (see FIG. 22).

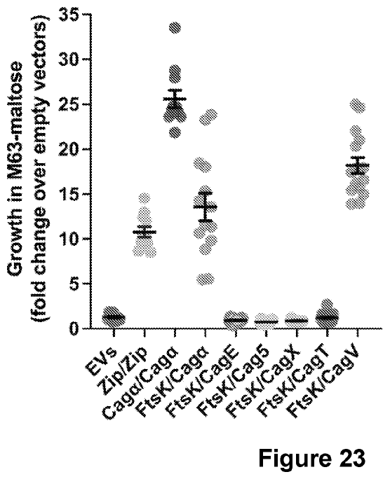

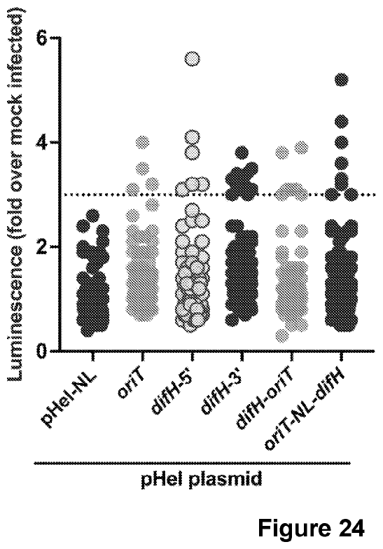

FtsK directly interacts with the cag T4SS apparatus, specifically Caga and the inner membrane component CagV, but not the core complex components CagX or CagT, or ATPases CagE and Cag5 (see FIG. 23). The present investigators have further shown that engineered plasmids may be translocated from H. pylori to target human cells via cag T4SS activity. See FIG. 24.

### REFERENCES

Ablasser, A., Bauernfeind, F., Hartmann, G., Latz, E., Fitzgerald, K.A., and Hornung, V. (2009). RIG-I-dependent sensing of poly(dA:dT) through the induction of an RNA polymerase III-transcribed RNA intermediate. Nature Immunol 10, 1065-1072.
Ablasser, A., Goldeck, M., Cavlar, T., Deimling, T., Witte, G., Rohl, I., Hopfner, K.P., Ludwig, J., and Hornung, V. (2013). cGAS produces a 2′-5′-linked cyclic dinucleotide second messenger that activates STING. Nature 498, 380-384.
Allan, C., Burel, J.M., Moore, J., Blackburn, C., Linkert, M., Loynton, S., Macdonald, D., Moore, W.J., Neves, C., Patterson, A., et al. (2012). OMERO: flexible, model-driven data management for experimental biology. Nat Methods 9, 245-253.
Almine, J.F., O'Hare, C.A., Dunphy, G., Haga, I.R., Naik, R.J., Atrih, A., Connolly, D.J., Taylor, J., Kelsall, I.R., Bowie, A.G., et al. (2017). IFI16 and cGAS cooperate in the activation of STING during DNA sensing in human keratinocytes. Nat Commm 8, 14392.
Amieva, M., and Peek, R.M. (2016). Pathobiology of Helicobacter pylori-induced gastric cancer. Gastroenterology 150, 64-78.
Bauer, M., Nascakova, Z., Mihai, A.I., Cheng, P.F., Levesque, M.P., Lampart, S., Hurwitz, R., Pfannkuch, L., Dobrovolna, J., Jacobs, M., et al. (2020). The ALPK1/TIFA/NF-kappaB axis links a bacterial carcinogen to R-loop-induced replication stress. Nat Commun 11, 5117.
Bebel, A., Karaca, E., Kumar, B., Stark, W.M., and Barabas, O. (2016). Structural snapshots of Xer recombination reveal activation by synaptic complex remodeling and DNA bending. Elife 5.
Bindels, D.S., Haarbosch, L., van Weeren, L., Postma, M., Wiese, K.E., Mastop, M., Aumonier, S., Gotthard, G., Royant, A., Hink, M.A., et al. (2017). mScarlet: a bright monomeric red fluorescent protein for cellular imaging. Nat Methods 14, 53-56.
Boxx, G.M., and Cheng, G. (2016). The roles of type I interferon in bacterial infection. Cell Host Microbe 19, 760-769.
Cai, X., Chiu, Y.H., and Chen, Z.J. (2014). The cGAS-cGAMP-STING pathway of cytosolic DNA sensing and signaling. Molecular Cell 54, 289-296.
Callaghan, M.M., Heilers, J.H., van der Does, C., and Dillard, J.P. (2017). Secretion of chromosomal DNA by the Neisseria gonorrhoeae type IV secretion system. Current Top Microbiol Immunol 413, 323-345.
Carozza, J.A., Bohnert, V., Nguyen, K.C., Skariah, G., Shaw, K.E., Brown, J.A., Rafat, M., von Eyben, R., Graves, E.E., Glenn, J.S., et al. (2020). Extracellular cGAMP is a cancer cell-produced immunotransmitter involved in radiation-induced anti-cancer immunity. Nat Cancer 1, 184-196.
Castano-Rodriguez, N., Kaakoush, N.O., and Mitchell, H.M. (2014). Pattern-recognition receptors and gastric cancer. Front Immunol 5, 336.
Chaturvedi, R., Asim, M., Piazuelo, M.B., Yan, F., Barry, D.P., Sierra, J.C., Delgado, A.G., Hill, S., Casero, R.A., Jr., Bravo, L.E., et al. (2014). Activation of EGFR and ERBB2 by Helicobacter pylori results in survival of gastric epithelial cells with DNA damage. Gastroenterology 146, 1739-1751 e1714.
Chen, S., Bonifati, S., Qin, Z., St Gelais, C., Kodigepalli, K.M., Barrett, B.S., Kim, S.H., Antonucci, J.M., Ladner, K.J., Buzovetsky, O., et al. (2018). SAMHD1 suppresses innate immune responses to viral infections and inflammatory stimuli by inhibiting the NF-kappaB and interferon pathways. Proc Nat Acad Sci USA 115, E3798-E3807.
Chiu, Y.H., Macmillan, J.B., and Chen, Z.J. (2009). RNA polymerase III detects cytosolic DNA and induces type I interferons through the RIG-I pathway. Cell 138, 576-591.
Chow, J., Franz, K.M., and Kagan, J.C. (2015). PRRs are watching you: Localization of innate sensing and signaling regulators. Virology 479-480, 104-109.
Christie, P.J., Atmakuri, K., Krishnamoorthy, V., Jakubowski, S., and Cascales, E. (2005). Biogenesis, architecture, and function of bacterial type IV secretion systems. Ann Rev Microbiol 59, 10.1146/annurev.micro.1158.030603.123630.
Chung, J.M., Sheedlo, M.J., Campbell, A.M., Sawhney, N., Frick-Cheng, A.E., Lacy, D.B., Cover, T.L., and Ohi, M.D. (2019). Structure of the Helicobacter pylori cag type IV secretion system. Elife 8.
Costa, T.R.D., Harb, L., Khara, P., Zeng, L., Hu, B., and Christie, P.J. (2021). Type IV secretion systems: advances in structure, function, and activation. Mol Microbiol 115, 436-452
Cover, T.L., Lacy, D.B., and Ohi, M.D. (2020). The Helicobacter pylori cag type IV secretion system. Trend Microbiol 28, 682-695.
Damke, P.P., Celma, L., Kondekar, S.M., Di Guilmi, A.M., Marsin, S., Depagne, J., Veaute, X., Legrand, P., Walbott, H., Vercruyssen, J., et al. (2022). ComFC mediates transport and handling of single-stranded DNA during natural transformation. Nat Commun 13, 1961.
Das, A., Dinh, P.X., Panda, D., and Pattnaik, A.K. (2014). Interferon-inducible protein IFI35 negatively regulates RIG-I antiviral signaling and supports vesicular stomatitis virus replication. J Virol 88, 3103-3113.
Debowski, A.W., Carnoy, C., Verbrugghe, P., Nilsson, H.O., Gauntlett, J.C., Fulurija, A., Camilleri, T., Berg, D.E., Marshall, B.J., and Benghezal, M. (2012). Xer recombinase and genome integrity in Helicobacter pylori, a pathogen without topoisomerase IV. PLoS One 7, e33310.
Diner, E.J., Burdette, D.L., Wilson, S.C., Monroe, K.M., Kellenberger, C.A., Hyodo, M., Hayakawa, Y., Hammond, M.C., and Vance, R.E. (2013). The innate immune DNA sensor cGAS produces a noncanonical cyclic dinucleotide that activates human STING. Cell Rep 3, 1355-1361.
Dixit, E., and Kagan, J.C. (2013). Intracellular pathogen detection by RIG-I-like receptors. Adv Immunol 117, 99-125.
Dooyema, S.D.R., Noto, J.M., Wroblewski, L.E., Piazuelo, M.B., Krishna, U., Suarez, G., Romero-Gallo, J., Delgado, A.G., and Peek, R.M. (2022). Helicobacter pylori actively suppresses innate immune nucleic acid receptors. Gut Microbes 14, 2105102.
Fernandez-Gonzalez, E., and Backert, S. (2014). DNA transfer in the gastric pathogen Helicobacter pylori. J Gastroenterol 49, 594-604.
Fernandez-Moreno, M., Hermida-Gomez, T., Gallardo, M.E., Dalmao-Fernandez, A., Rego-Perez, I., Garesse, R., and Blanco, F.J. (2016). Generating Rho-0 cells using mesenchymal stem cell lines. PLoS One 11, e0164199.
Fischer, W., Tegtmeyer, N., Stingl, K., and Backert, S. (2020). Four chromosomal type IV secretion systems in Helicobacter pylori:composition, structure and function. Front Microbiol 11, 1592.
Frick-Cheng, A.E., Pyburn, T.M., Voss, B.J., McDonald, W.H., Ohi, M.D., and Cover, T.L. (2016). Molecular and structural analysis of the Helicobacter pylori cag type IV secretion system core complex. mBio 7.
Gall, A., Gaudet, R.G., Gray-Owen, S.D., and Salama, N.R. (2017). TIFA signaling in gastric epithelial cells initiates the cag type 4 secretion system-dependent innate immune response to Helicobacter pylori infection. mBio 8.
Gao, D., Li, T., Li, X.D., Chen, X., Li, Q.Z., Wight-Carter, M., and Chen, Z.J. (2015). Activation of cyclic GMP-AMP synthase by self-DNA causes autoimmune diseases. Proc Nat Acad Sci USA 112, E5699-5705.
Gao, P., Ascano, M., Wu, Y., Barchet, W., Gaffney, B.L., Zillinger, T., Serganov, A.A., Liu, Y., Jones, R.A., Hartmann, G., et al. (2013a). Cyclic [G(2′,5′)pA(3′,5′)p] is the metazoan second messenger produced by DNA-activated cyclic GMP-AMP synthase. Cell 153, 1094-1107.
Gao, P., Ascano, M., Zillinger, T., Wang, W., Dai, P., Serganov, A.A., Gaffney, B.L., Shuman, S., Jones, R.A., Deng, L., et al. (2013b). Structure-function analysis of STING activation by c[G(2′,5′)pA(3′,5′)p] and targeting by antiviral DMXAA. Cell 154, 748-762.
Garcia-Martinez, I., Santoro, N., Chen, Y., Hoque, R., Ouyang, X., Caprio, S., Shlomchik, M.J., Coffman, R.L., Candia, A., and Mehal, W.Z. (2016). Hepatocyte mitochondrial DNA drives nonalcoholic steatohepatitis by activation of TLR9. J Clin Invest 126, 859-864.
Grove, J.I., Alandiyjany, M.N., and Delahay, R.M. (2013). Site-specific relaxase activity of a VirD2-like protein encoded within the tfs4 genomic island of Helicobacter pylori. J Biol Chem 288, 26385-26396.
Hanada, K., Uchida, T., Tsukamoto, Y., Watada, M., Yamaguchi, N., Yamamoto, K., Shiota, S., Moriyama, M., Graham, D.Y., and Yamaoka, Y. (2014). Helicobacter pylori infection introduces DNA double-strand breaks in host cells. Infect Immun 82, 4182-4189.
Ishii, K.J., Coban, C., Kato, H., Takahashi, K., Torii, Y., Takeshita, F., Ludwig, H., Sutter, G., Suzuki, K., Hemmi, H., et al. (2006). A Toll-like receptor-independent antiviral response induced by double-stranded B-form DNA. Nat Immunol 7, 40-48.
Ishikawa, H., and Barber, G.N. (2008). STING is an endoplasmic reticulum adaptor that facilitates innate immune signalling. Nature 455, 674-678.
Ishikawa, H., Ma, Z., and Barber, G.N. (2009). STING regulates intracellular DNA-mediated, type I interferon-dependent innate immunity. Nature 461, 788-792.
Johnson, E.M., Gaddy, J.A., Voss, B.J., Hennig, E.E., and Cover, T.L. (2014). Genes required for assembly of pili associated with the Helicobacter pylori cag type IV secretion system. Infect Immun 82, 3457-70.
Karimova, G., Gauliard, E., Davi, M., Ouellette, S.P., and Ladant, D. (2017). Protein-protein interaction: bacterial two-hybrid. Methods Mol Biol 1615, 159-176.
Kuriakose, T., Man, S.M., Malireddi, R.K., Karki, R., Kesavardhana, S., Place, D.E., Neale, G., Vogel, P., and Kanneganti, T.D. (2016). ZBP1/DAI is an innate sensor of influenza virus triggering the NLRP3 inflammasome and programmed cell death pathways. Sci Immunol 1.
Lin, A.S., Dooyema, S.D.R., Frick-Cheng, A.E., Harvey, M.L., Suarez, G., Loh, J.T., McDonald, W.H., McClain, M.S., Peek, R.M., Jr., and Cover, T.L. (2020). Bacterial energetic requirements for Helicobacter pylori cag type IV secretion system-dependent alterations in gastric epithelial cells. Infect Immun 88, e00790-19.
Marinho, F.V., Benmerzoug, S., Oliveira, S.C., Ryffel, B., and Quesniaux, V.F.J. (2017). The emerging roles of STING in bacterial infections. Trend Microbiol 25, 906-918.
McArthur, K., Whitehead, L.W., Heddleston, J.M., Li, L., Padman, B.S., Oorschot, V., Geoghegan, N.D., Chappaz, S., Davidson, S., San Chin, H., et al. (2018). BAK/BAX macropores facilitate mitochondrial herniation and mtDNA efflux during apoptosis. Science 359.
Miao, L., Qi, J., Zhao, Q., Wu, Q.N., Wei, D.L., Wei, X.L., Liu, J., Chen, J., Zeng, Z.L., Ju, H.Q., et al. (2020). Targeting the STING pathway in tumor-associated macrophages regulates innate immune sensing of gastric cancer cells. Theranostics 10, 498-515.
Mortier-Barriere, I., Velten, M., Dupaigne, P., Mirouze, N., Pietrement, O., McGovern, S., Fichant, G., Martin, B., Noirot, P., Le Cam, E., et al. (2007). A key presynaptic role in transformation for a widespread bacterial protein: DprA conveys incoming ssDNA to RecA. Cell 130, 824-836.
Nandakumar, R., Tschismarov, R., Meissner, F., Prabakaran, T., Krissanaprasit, A., Farahani, E., Zhang, B.C., Assil, S., Martin, A., Bertrams, W., et al. (2019). Intracellular bacteria engage a STING-TBK1-MVB12b pathway to enable paracrine cGAS-STING signalling. Nat Microbiol 4, 701-713.
Oka, T., Hikoso, S., Yamaguchi, O., Taneike, M., Takeda, T., Tamai, T., Oyabu, J., Murakawa, T., Nakayama, H., Nishida, K., et al. (2012). Mitochondrial DNA that sscapes from autophagy causes inflammation and heart failure. Nature 485, 251-255.
Onomoto, K., Onoguchi, K., and Yoneyama, M. (2021). Regulation of RIG-I-like receptor-mediated signaling: interaction between host and viral factors. Cell Mol Immunol 18, 539-555.
Peignier, A., and Parker, D. (2021). Impact of type I interferons on susceptibility to bacterial pathogens. Trend Microbiol 29, 823-835.
Qiao, Y., Zhu, S., Deng, S., Zou, S.S., Gao, B., Zang, G., Wu, J., Jiang, Y., Liu, Y.J., and Chen, J. (2020). Human cancer cells sense cytosolic nucleic acids through the RIG-I-MAVS pathway and cGAS-STING pathway. Front Cell Dev Biol 8, 606001.
Rad, R., Ballhorn, W., Voland, P., Eisenacher, K., Mages, J., Rad, L., Ferstl, R., Lang, R., Wagner, H., Schmid, R.M., et al. (2009). Extracellular and intracellular pattern recognition receptors cooperate in the recognition of Helicobacter pylori. Gastroenterology 136, 2247-2257.
Rathinam, V.A., Jiang, Z., Waggoner, S.N., Sharma, S., Cole, L.E., Waggoner, L., Vanaja, S.K., Monks, B.G., Ganesan, S., Latz, E., et al. (2010). The AIM2 inflammasome is essential for host defense against cytosolic bacteria and DNA viruses. Nat Immunol 11, 395-402.
Rongvaux, A., Jackson, R., Harman, C.C., Li, T., West, A.P., de Zoete, M.R., Wu, Y., Yordy, B., Lakhani, S.A., Kuan, C.Y., et al. (2014). Apoptotic caspases prevent the induction of type I interferons by mitochondrial DNA. Cell 159, 1563-1577.
Salama, N.R., Hartung, M.L., and Muller, A. (2013). Life in the human stomach: persistence strategies of the bacterial pathogen Helicobacter pylori. Nat Rev Microbiol 11, 385-399.
Savvides, S.N., Yeo, H.J., Beck, M.R., Blaesing, F., Lurz, R., Lanka, E., Buhrdorf, R., Fischer, W., Haas, R., and Waksman, G. (2003). VirB11 ATPases are dynamic hexameric assemblies: new insights into bacterial type IV secretion. EMBO J 22, 1969-1980.
Schwartz, S.L., Park, E.N., Vachon, V.K., Danzy, S., Lowen, A.C., and Conn, G.L. (2020). Human OASI activation is highly dependent on both RNA sequence and context of activating RNA motifs. Nucleic Acids Res 48, 7520-7531.
Shaffer, C.L., Gaddy, J.A., Loh, J.T., Johnson, E.M., Hill, S., Hennig, E.E., McClain, M.S., McDonald, W.H., and Cover, T.L. (2011). Helicobacter pylori exploits a unique repertoire of type IV secretion system components for pilus assembly at the bacteria-host cell interface. PLoS Pathog 7, e1002237.
Song, S., Peng, P., Tang, Z., Zhao, J., Wu, W., Li, H., Shao, M., Li, L., Yang, C., Duan, F., et al. (2017). Decreased expression of STING predicts poor prognosis in patients with gastric cancer. Sci Rep 7, 39858.
Stein, S.C., Faber, E., Bats, S.H., Murillo, T., Speidel, Y., Coombs, N., and Josenhans, C. (2017). Helicobacter pylori modulates host cell responses by cagT4SS-dependent translocation of an intermediate metabolite of LPS inner core heptose biosynthesis. PLoS Pathog 13, e1006514.
Stetson, D.B., and Medzhitov, R. (2006). Recognition of cytosolic DNA activates an IRF3-dependent innate immune response. Immunity 24, 93-103.
Storek, K.M., Gertsvolf, N.A., Ohlson, M.B., and Monack, D.M. (2015). cGAS and Ifi204 cooperate to produce type I IFNs in response to Francisella infection. J Immunol 194, 3236-3245.
Sun, L., Wu, J., Du, F., Chen, X., and Chen, Z.J. (2013). Cyclic GMP-AMP synthase is a cytosolic DNA sensor that activates the type I interferon pathway. Science 339, 786-791.
Sun, W., Li, Y., Chen, L., Chen, H., You, F., Zhou, X., Zhou, Y., Zhai, Z., Chen, D., and Jiang, Z. (2009). ERIS, an endoplasmic reticulum IFN stimulator, activates innate immune signaling through dimerization. Proc Nat Acad Sci USA 106, 8653-8658.
Sung, H., Ferlay, J., Siegel, R.L., Laversanne, M., Soerjomataram, I., Jemal, A., and Bray, F. (2021). Global Cancer Statistics 2020: GLOBOCAN estimates of incidence and mortality worldwide for 36 cancers in 185 countries. CA Cancer J Clin 71, 209-249.
Tegtmeyer, N., Linz, B., Yamaoka, Y., and Backert, S. (2022). Unique TLR9 activation by Helicobacter pylori depends on the cag T4SS, but not on VirD2 relaxases or VirD4 coupling proteins. Current Microbiol 79, 121.
Unterholzner, L., Keating, S.E., Baran, M., Horan, K.A., Jensen, S.B., Sharma, S., Sirois, C.M., Jin, T., Latz, E., Xiao, T.S., et al. (2010). IFI16 is an innate immune sensor for intracellular DNA. Nat Immunol 11, 997-1004.
Varga, M.G., Piazuelo, M.B., Romero-Gallo, J., Delgado, A.G., Suarez, G., Whitaker, M.E., Krishna, U.S., Patel, R. V., Skaar, E.P., Wilson, K.T., et al. (2016a). TLR9 activation suppresses inflammation in response to Helicobacter pylori infection. Am J Physiol Gastrointest Liver Physiol 311, G852-G858.
Varga, M.G., Shaffer, C.L., Sierra, J.C., Suarez, G., Piazuelo, M.B., Whitaker, M.E., Romero-Gallo, J., Krishna, U.S., Delgado, A., Gomez, M.A., et al. (2016b). Pathogenic Helicobacter pylori strains translocate DNA and activate TLR9 via the cancer-associated cag type IV secretion system. Oncogene 35, 6262-6269.
Varga, M.G., Wood, C.R., Butt, J., Ryan, M.E., You, W.C., Pan, K., Waterboer, T., Epplein, M., and Shaffer, C.L. (2021). Immunostimulatory membrane proteins potentiate H. pylori-induced carcinogenesis by enabling CagA translocation. Gut Microbes 13, 1-13.
Viala, J., Chaput, C., Boneca, I.G., Cardona, A., Girardin, S.E., Moran, A.P., Athman, R., Memet, S., Huerre, M.R., Coyle, A.J., et al. (2004). Nod1 responds to peptidoglycan delivered by the Helicobacter pylori cag pathogenicity island. Nat Immunol 5, 1166-1174.
Watanabe, T., Asano, N., Fichtner-Feigl, S., Gorelick, P.L., Tsuji, Y., Matsumoto, Y., Chiba, T., Fuss, I.J., Kitani, A., and Strober, W. (2010). NOD1 contributes to mouse host defense against Helicobacter pylori via induction of type I IFN and activation of the ISGF3 signaling pathway. J Clin Invest 120, 1645-1662.
Watson, R.O., Bell, S.L., MacDuff, D.A., Kimmey, J.M., Diner, E.J., Olivas, J., Vance, R.E., Stallings, C.L., Virgin, H.W., and Cox, J.S. (2015). The cytosolic sensor cGAS detects Mycobacterium tuberculosis DNA to induce type I interferons and activate autophagy. Cell Host Microbe 17, 811-819.
West, A.P., Khoury-Hanold, W., Staron, M., Tal, M.C., Pineda, C.M., Lang, S.M., Bestwick, M., Duguay, B.A., Raimundo, N., MacDuff, D.A., et al. (2015). Mitochondrial DNA stress primes the antiviral innate immune response. Nature 520, 553-557.
White, M.J., McArthur, K., Metcalf, D., Lane, R.M., Cambier, J.C., Herold, M.J., van Delft, M.F., Bedoui, S., Lessene, G., Ritchie, M.E., et al. (2014). Apoptotic caspases suppress mtDNA-induced STING-mediated type I IFN production. Cell 159, 1549-1562.
Wu, J., Sun, L., Chen, X., Du, F., Shi, H., Chen, C., and Chen, Z.J. (2013). Cyclic GMP-AMP is an endogenous second messenger in innate immune signaling by cytosolic DNA. Science 339, 826-830.
Wu, X.M., Zhang, J., Li, P.W., Hu, Y.W., Cao, L., Ouyang, S., Bi, Y.H., Nie, P., and Chang, M.X. (2020). NOD1 promotes antiviral signaling by binding viral RNA and regulating the interaction of MDA5 and MAVS. J Immunol 204, 2216-2231.
Zhang, Q., Raoof, M., Chen, Y., Sumi, Y., Sursal, T., Junger, W., Brohi, K., Itagaki, K., and Hauser, C.J. (2010). Circulating mitochondrial DAMPs cause inflammatory responses to injury. Nature 464, 104-107.
Zhang, X., Bogunovic, D., Payelle-Brogard, B., Francois-Newton, V., Speer, S.D., Yuan, C., Volpi, S., Li, Z., Sanal, O., Mansouri, D., et al. (2015). Human intracellular ISG15 prevents interferon-alpha/beta over-amplification and auto-inflammation. Nature 517, 89-93.
Zhang, X., Shi, H., Wu, J., Zhang, X., Sun, L., Chen, C., and Chen, Z.J. (2013). Cyclic GMP-AMP containing mixed phosphodiester linkages is an endogenous high-affinity ligand for STING. Mol Cell 51, 226-235.
Zhang, Y., Yeruva, L., Marinov, A., Prantner, D., Wyrick, P.B., Lupashin, V., and Nagarajan, U.M. (2014). The DNA sensor, cyclic GMP-AMP synthase, is essential for induction of IFN-beta during Chlamydia trachomatis infection. J Immunol 193, 2394-2404.
Zhong, B., Yang, Y., Li, S., Wang, Y.Y., Li, Y., Diao, F., Lei, C., He, X., Zhang, L., Tien, P., et al. (2008). The adaptor protein MITA links virus-sensing receptors to IRF3 transcription factor activation. Immunity 29, 538-550.
Zimmermann, S., Pfannkuch, L., Al-Zeer, M.A., Bartfeld, S., Koch, M., Liu, J., Rechner, C., Soerensen, M., Sokolova, O., Zamyatina, A., et al. (2017). ALPK1- and TIFA-dependent innate immune response triggered by the Helicobacter pylori type IV secretion system. Cell Rep 20, 2384-2395.
1 Chung, J. M. et al. Structure of the Helicobacter pylori Cag type IV secretion system. eLife 8 (2019)
2 Durie, C. L. et al. Structural analysis of the Legionella pneumophila Dot/Icm type IV secretion system core complex. eLife 9 (2020).
3 Sheedlo, M. J. et al. Cryo-EM reveals species-specific components within the Helicobacter pylori Cag type IV secretion system core complex. eLife 9 (2020).
4 Sheedlo, M. J., Ohi, M. D., Lacy, D. B. & Cover, T. L. Molecular architecture of bacterial type IV secretion systems. PLoS Pathog 18, e1010720 (2022).
5 Chernyatina, A. A. & Low, H. H. Core architecture of a bacterial type II secretion system. Nat Commun 10, 5437 (2019).
6 Hu, J. et al. Cryo-EM analysis of the T3S injectisome reveals the structure of the needle and open secretin. Nat Commun 9, 3840 (2018).
7 Johnson, S. et al. Symmetry mismatch in the MS-ring of the bacterial flagellar rotor explains the structural coordination of secretion and rotation. Nat Microbiol 5, 966-975 (2020).
8 Dix, S. R. et al. Structural insights into the function of type VI secretion system TssA subunits. Nat Commun 9, 4765 (2018).
9 Macé, K. et al. Cryo-EM structure of a type IV secretion system. Nature 607, 191-196 (2022).
10 Christie, P. J., Atmakuri, K., Krishnamoorthy, V., Jakubowski, S. & Cascales, E. Biogenesis, architecture, and function of bacterial type IV secretion systems. Ann Rev Microbiol 59, 451-85 (2005).
11 Costa, T. R. D. et al. Type IV secretion systems: Advances in structure, function, and activation. Mol Microbiol 115, 436-452 (2021).
12 Gordon, J. E. et al. Use of chimeric type IV secretion systems to define contributions of outer membrane subassemblies for contact-dependent translocation. Mol Microbiol 105, 273-293 (2017).
13 Chandran Darbari, V. & Waksman, G. Structural biology of bacterial type IV secretion systems. Annu Rev Biochem 84, 603-629 (2015).
14 Rivera-Calzada, A. et al. Structure of a bacterial type IV secretion core complex at subnanometre resolution. EMBO J 32, 1195-1204 (2013).
15 Low, H. H. et al. Structure of a type IV secretion system. Nature 508, 550-553 (2014).
16 Atmakuri, K., Cascales, E. & Christie, P. J. Energetic components VirD4, VirB11 and VirB4 mediate early DNA transfer reactions required for bacterial type IV secretion. Mol Microbiol 54, 1199-1211 (2004).
17 Hu, B. et al. In situ molecular architecture of the Helicobacter pylori Cag type IV secretion system. mBio 10 (2019).
18 Lin, A. S. et al. Bacterial energetic requirements for Helicobacter pylori Cag type IV secretion system-dependent alterations in gastric epithelial cells. Infect Immun 88 (2020)
19 Redzej, A. et al. Structure of a VirD4 coupling protein bound to a VirB type IV secretion machinery. EMBO J (2017).
20 Bhatty, M., Laverde Gomez, J. A. & Christie, P. J. The expanding bacterial type IV secretion lexicon. Res Microbiol 164, 620-639 (2013).
21 Ryan, M. E., Damke, P. P. & Shaffer, C. L. DNA transport through the dynamic type IV secretion system. Infect Immun, e0043622 (2023).
22 Alvarez-Martinez, C. E. & Christie, P. J. Biological diversity of prokaryotic type IV secretion systems. Microbiol Mol Biol Rev 73, 775-808 (2009).
23 Cascales, E. & Christie, P. J. The versatile bacterial type IV secretion systems. Nat Rev Microbiol 1, 137-149 (2003).
24 Cascales, E. & Christie, P. J. Definition of a bacterial type IV secretion pathway for a DNA substrate. Science 304, 1170-1173 (2004).
25 Grohmann, E., Christie, P. J., Waksman, G. & Backert, S. Type IV secretion in Gram-negative and Gram-positive bacteria. Mol Microbiol 107, 455-471 (2018).
26 Lacroix, B. & Citovsky, V. A Functional bacterium-to-plant DNA transfer machinery of Rhizobium etli. PLoS Pathog 12, e1005502 (2016).
27 Schroder, G., Schuelein, R., Quebatte, M. & Dehio, C. Conjugative DNA transfer into human cells by the VirB/VirD4 type IV secretion system of the bacterial pathogen Bartonella henselae. Proc Natl Acad Sci U S A 108, 14643-14648 (2011).
28 Alperi, A. et al. A translocation motif in relaxase TrwC specifically affects recruitment by its conjugative type IV secretion system. J Bacteriol 195, 4999-5006 (2013).
29 Guzmán-Herrador, D. L. et al. DNA delivery and genomic integration into mammalian target cells through type IV A and B secretion systems of human pathogens. Front Microbiol 8, 1503 (2017).
30 González-Prieto, C., Gabriel, R., Dehio, C., Schmidt, M. & Llosa, M. The conjugative relaxase TrwC promotes integration of foreign DNA in the human genome. Appl Environ Microbiol 83 (2017).
31 Nagai, H. & Kubori, T. Type IVB secretion systems of Legionella and other Gram-negative bacteria. Front Microbiol 2, 136 (2011).
32 Vogel, J. P., Andrews, H. L., Wong, S. K. & Isberg, R. R. Conjugative transfer by the virulence system of Legionella pneumophila. Science 279, 873-876 (1998).
33 Varga, M. G. et al. Pathogenic Helicobacter pylori strains translocate DNA and activate TLR9 via the cancer-associated cag type IV secretion system. Oncogene (2016).
34 Damke, P. P., Wood, C. R. & Shaffer, C. L. Helicobacter pylori provokes STING immunosurveillance via trans-kingdom conjugation. bioRxiv, 2022.2006.2029.498044 (2022).
35 Varga, M. G. et al. TLR9 activation suppresses inflammation in response to Helicobacter pylori infection. Am J Physiol Gastrointest Liver Physiol 311, G852-G858 (2016)
36 Dooyema, S. D. R. et al. Helicobacter pylori actively suppresses innate immune nucleic acid receptors. Gut Microbes 14, 2105102 (2022).
37 Chang, Y. W., Shaffer, C. L., Rettberg, L. A., Ghosal, D. & Jensen, G. J. In vivo structures of the Helicobacter pylori cag type IV secretion system. Cell Rep 23, 673-681 (2018).
38 Frick-Cheng, A. E. et al. Molecular and structural analysis of the Helicobacter pylori cag type IV secretion system core complex. mBio 7 (2016).
39 Sheedlo, M. J. et al. Cryo-EM reveals new species-specific proteins and symmetry elements in the Legionella pneumophila Dot/Icm T4SS. eLife 10, e70427 (2021).
40 Amin, H., Ilangovan, A. & Costa, T. R. D. Architecture of the outer-membrane core complex from a conjugative type IV secretion system. Nat Commun 12, 6834 (2021)
41 Liu, X., Khara, P., Baker, M. L., Christie, P. J. & Hu, B. Structure of a type IV secretion system core complex encoded by multi-drug resistance F plasmids. Nat Commun 13, 379 (2022).
42 Li, Y. G., Hu, B. & Christie, P. J. Biological and structural diversity of type IV secretion systems. Microbiol Spectr 7 (2019).
43 Backert, S., Tegtmeyer, N. & Fischer, W. Composition, structure and function of the Helicobacter pylori cag pathogenicity island encoded type IV secretion system. Future Microbiol 10, 955-965 (2015).
44 Fischer, W., Tegtmeyer, N., Stingl, K. & Backert, S. Four chromosomal type IV secretion systems in Helicobacter pylori:Composition, structure and function. Front Microbiol 11, 1592 (2020).
45 Jakubowski, S. J. et al. Agrobacterium VirB10 domain requirements for type IV secretion and T pilus biogenesis. Mol Microbiol 71, 779-794 (2009)
46 Sagulenko, E., Sagulenko, V., Chen, J. & Christie, P. J. Role of Agrobacterium VirB11 ATPase in T-pilus assembly and substrate selection. J Bacteriol 183, 5813-5825 (2001).
47 Fischer, W. et al. Systematic mutagenesis of the Helicobacter pylori cag pathogenicity island: essential genes for CagA translocation in host cells and induction of interleukin-8. Mol Microbiol 42, 1337-1348 (2001).
48 Shaffer, C. L. et al. Helicobacter pylori exploits a unique repertoire of type IV secretion system components for pilus assembly at the bacteria-host cell interface. PLoS Pathog 7, e1002237 (2011).
49 Varga, M. G. et al. Immunostimulatory membrane proteins potentiate H. pylori-induced carcinogenesis by enabling CagA translocation. Gut Microbes 13, 1-13 (2021).
50 Banta, L. M. et al. An Agrobacterium VirB10 mutation conferring a type IV secretion system gating defect. J Bacteriol 193, 2566-2574 (2011).
51 Volkan, E. et al. Molecular basis of usher pore gating in Escherichia coli pilus biogenesis. Proc Natl Acad Sci U S A 110, 20741-20746 (2013).
52 Mao, J. C. & Putterman, M. Accumulation in gram-postive and gram-negative bacteria as a mechanism of resistance to erythromycin. J Bacteriol 95, 1111-1117 (1968).
53 Andrzejewska, J. et al. Characterization of the pilin ortholog of the Helicobacter pylori type IV cag pathogenicity apparatus, a surface-associated protein expressed during infection. J Bacteriol 188, 5865-5877 (2006).
54 Jumper, J. et al. Highly accurate protein structure prediction with AlphaFold. Nature 596, 583-589 (2021).
55 Mirdita, M. et al. ColabFold—Making protein folding accessible to all. bioRxiv, 2021.2008.2015.456425 (2021).
56 Varadi, M. et al. AlphaFold protein structure database: massively expanding the structural coverage of protein-sequence space with high-accuracy models. Nucleic Acids Res 50, D439-d444 (2022).
57 Kreida, S. et al. Cryo-EM structure of the Agrobacterium tumefaciens T4SS-associated T-pilus reveals stoichiometric protein-phospholipid assembly. Structure 31, 385-394.e384 (2023).
58 Amro, J. et al. Cryo-EM structure of the Agrobacterium tumefaciens T-pilus reveals the importance of positive charges in the lumen. Structure 31, 375-384.e374 (2023).
59 Costa, T. R. D. et al. Structure of the bacterial sex F pilus reveals an assembly of a stoichiometric protein-phospholipid complex. Cell 166, 1436-1444 e1410 (2016)
60 Eisenbrandt, R. et al. Conjugative pili of IncP plasmids, and the Ti plasmid T pilus are composed of cyclic subunits. J Biol Chem 274, 22548-22555 (1999).
61 Sivanesan, D., Hancock, M. A., Villamil Giraldo, A. M. & Baron, C. Quantitative analysis of VirB8-VirB9-VirB10 interactions provides a dynamic model of type IV secretion system core complex assembly. Biochemistry 49, 4483-4493 (2010).
62 Karimova, G., Gauliard, E., Davi, M., Ouellette, S. P. & Ladant, D. Protein-protein interaction: Bacterial two-hybrid. Met Mol Biol 1615, 159-176 (2017).
63 Gunton, J. E., Gilmour, M. W., Alonso, G. & Taylor, D. E. Subcellular localization and functional domains of the coupling protein, TraG, from IncHI1 plasmid R27. Microbiology 151, 3549-3561 (2005).
64 Mortier-Barriere, I. et al. A key presynaptic role in transformation for a widespread bacterial protein: DprA conveys incoming ssDNA to RecA. Cell 130, 824-836 (2007).
65 Pattis, I., Weiss, E., Laugks, R., Haas, R. & Fischer, W. The Helicobacter pylori CagF protein is a type IV secretion chaperone-like molecule that binds close to the C-terminal secretion signal of the CagA effector protein. Microbiology 153, 2896-2909 (2007).
66 Jakubowski, S. J., Cascales, E., Krishnamoorthy, V. & Christie, P. J. Agrobacterium tumefaciens VirB9, an outer-membrane-associated component of a type IV secretion system, regulates substrate selection and T-pilus biogenesis. J Bacteriol 187, 3486-3495 (2005).
67 Garza, I. & Christie, P. J. A putative transmembrane leucine zipper of Agrobacterium VirB10 is essential for T-pilus biogenesis but not type IV secretion. J Bacteriol 195, 3022-3034 (2013).
68 Barrozo, R. M. et al. Functional plasticity in the type IV secretion system of Helicobacter pylori. PLoS Pathog 9, e1003189 (2013).
69 Johnson, E. M., Gaddy, J. A., Voss, B. J., Hennig, E. E. & Cover, T. L. Genes required for assembly of pili associated with the Helicobacter pylori cag type IV secretion system. Infect Immun (2014).
70 Kwok, T. et al. Helicobacter exploits integrin for type IV secretion and kinase activation. Nature 449, 862-866 (2007).
71 Hu, B., Khara, P. & Christie, P. J. Structural bases for F plasmid conjugation and F pilus biogenesis in Escherichia coli. Proc Natl Acad Sci U S A 116, 14222-14227 (2019).
72 Hu, B., Lara-Tejero, M., Kong, Q., Galan, J. E. & Liu, J. In situ molecular architecture of the Salmonella type III secretion machine. Cell 168, 1065-1074 e1010 (2017).
73 Barden, S. et al. A helical RGD motif promoting cell adhesion: crystal structures of the Helicobacter pylori type IV secretion system pilus protein CagL. Structure 21, 1931-1941 (2013).
74 Barden, S. et al. Structure of a three-dimensional domain-swapped dimer of the Helicobacter pylori type IV secretion system pilus protein CagL. Acta Crystallogr B Struct 70, 1391-1400 (2014).
75 Dutka, P. et al. Structure and function of the Dot/Icm T4SS. bioRxiv, 2023.2003.2022.533729 (2023).
76 Ghosal, D., Chang, Y. W., Jeong, K. C., Vogel, J. P. & Jensen, G. J. In situ structure of the Legionella Dot/Icm type IV secretion system by electron cryotomography. EMBO Rep 18, 726-732 (2017).
77 Ghosal, D. et al. Molecular architecture, polar targeting and biogenesis of the Legionella Dot/Icm T4SS. Nat Microbiol 4, 1173-1182 (2019).
78 Kaplan, M. et al. In situ imaging of bacterial outer membrane projections and associated protein complexes using electron cryo-tomography. eLife 10, e73099 (2021).
79 Schindelin, J. et al. Fiji: an open-source platform for biological-image analysis. Nat Methods 9, 676-682 (2012).
80 Benoit, S. L., Agudelo, S. & Maier, R. J. A two-hybrid system reveals previously uncharacterized protein-protein interactions within the Helicobacter pylori NIF iron-sulfur maturation system. Sci Rep 11, 10794 (2021).
81 Benoit, S. L., Holland, A. A., Johnson, M. K. & Maier, R. J. Iron-sulfur protein maturation in Helicobacter pylori:identifying a Nfu-type cluster carrier protein and its iron-sulfur protein targets. Mol Microbiol 108, 379-396 (2018).
82 Damke, P. P. et al. ComFC mediates transport and handling of single-stranded DNA during natural transformation. Nat Commun 13, 1961 (2022).
83 Grove, J. I., Alandiyjany, M. N. & Delahay, R. M. Site-specific relaxase activity of a VirD2-like protein encoded within the tfs4 genomic island of Helicobacter pylori. J Biol Chem 288, 26385-26396 (2013).
84 Pettersen, E. F. et al. UCSF ChimeraX: Structure visualization for researchers, educators, and developers. Protein Sci 30, 70-82 (2021).

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

1. A system for delivering a substrate to a target host cell, comprising:
a synthetic Helicobacter pylori cag type IV secretion system (cag T4SS) synthesized within an avirulent bacterial cell; and one or more bioactive molecules of interest.

2. The system of claim 1, comprising one or more artificial Helicobacter pylori cag T4SS operons selected from the group consisting of: CagA, CagC, CadD, CagE, CagF, CagG, CagH, CagI, CagM, CagN, CagO, CagP, CagQ, CagS, CagT, CagU, CagV, CagW, CagX, CagY, CagZ, Cag1, Cag2, Cag3, Cag4, Cag5, Cag-alpha.

3. The system of claim 1, wherein the one or more artificial T4SS operons introduced into the avirulent bacterial cell result in a self-assembling, bi-membrane spanning secretion channel complex adapted to translocate the bioactive molecule to the target host cell.

4. The system of claim 1, wherein the bioactive molecule is a polynucleotide or protein of interest.

5. The system of claim 4, wherein the polynucleotide or protein of interest is a DNA or an RNA.

6. The system of claim 1, wherein the bioactive molecule is one or more DNA-encoded vaccine antigens.

7. The system of claim 1, wherein the bioactive molecule is a DNA-encoded Cas protein and one or more guide RNAs.

8. The system of claim 1, wherein the bioactive molecule is one or more bacterial antigens.

9. The system of claim 1, wherein the bioactive molecule is an immunostimulatory bacterial DNA adjuvant.

10. The system of claim 1, wherein the bioactive molecule is a modified bacterial peptidoglycan.

11. The system of claim 1, wherein the bioactive molecule is a bacterial-derived cyclic signaling molecule.

12. The system of claim 1, wherein the bioactive molecule is a DNA-RNA hybrid.

13. The system of claim 2, wherein the one or more artificial T4SS operons introduced into the avirulent bacterial cell result in a self-assembling, bi-membrane spanning secretion channel complex adapted to translocate the bioactive molecule to the target host cell.

14. The system of claim 2, wherein the bioactive molecule is one or more DNA-encoded vaccine antigens.

15. The system of claim 2, wherein the bioactive molecule is a DNA-encoded Cas protein and one or more guide RNAs.

16. The system of claim 2, wherein the bioactive molecule is one or more bacterial antigens.

17. The system of claim 2, wherein the bioactive molecule is an immunostimulatory bacterial DNA adjuvant.

18. The system of claim 2, wherein the bioactive molecule is a modified bacterial peptidoglycan.

19. The system of claim 2, wherein the bioactive molecule is a bacterial-derived cyclic signaling molecule.

20. The system of claim 2, wherein the bioactive molecule is a DNA-RNA hybrid.
